(19) United States
(12) Patent Application Publication
(10) Pub. No.: US 2024/0245614 A1
(43) Pub. Date: Jul. 25, 2024

(54) PRODUCTION OF THERAPEUTIC MESENCHYMAL STEM CELL-DERIVED EXOSOMES

(71) Applicant: BOARD OF REGENTS, THE UNIVERSITY OF TEXAS SYSTEM, Austin, TX (US)

(72) Inventors: Frederick F. LANG, Houston, TX (US); Elizabeth J. SHPALL, Houston, TX (US); Katy REZVANI, Houston, TX (US); Mayela MENDT, Houston, TX (US); Daniel LEDBETTER, Houston, TX (US); Joy GUMIN, Houston, TX (US); Brittany PARKER KERRIGAN, Houston, TX (US); Anwar HOSSAIN, Houston, TX (US); Frederick M. LANG, Houston, TX (US)

(73) Assignee: BOARD OF REGENTS, THE UNIVERSITY OF TEXAS SYSTEM, Austin, TX (US)

(21) Appl. No.: 18/559,667

(22) PCT Filed: May 17, 2022
(86) PCT No.: PCT/US2022/029684

§ 371 Date: Nov. 8, 2023

Related U.S. Application Data
(60) Provisional application No. 63/191,202, filed on May 20, 2021.

(51) Int. Cl. A61K 9/50; A61K 9/00; A61P 35/00; C12N 5/0775; C12N 15/113

(52) U.S. Cl. CPC A61K 9/5068; A61K 9/0019; A61K 9/0085; A61P 35/00; C12N 5/0665; C12N 15/1135; C12N 15/1138; C12N 2310/141; C12N 2501/65; C12N 2510/02

(57) ABSTRACT

Aspects of the disclosure encompass systems, methods, and compositions for producing exosomes from mesenchymal stem cells and, optionally, loading said exosomes with one or more therapeutic agents. The systems, methods, and compositions may occur in an automated cell expansion system that allows for controllable parameters and from which cells and exosomes may be harvested at one or more times as part of a particular regimen. The exosomes may be loaded with one or more therapeutic agents using electroporation. In specific aspects, the exosomes may be provided to an individual in need thereof, including in some cases where the individual in need thereof is an individual having a medical disorder for which the exosomes would be therapeutic.

### CROSS-REFERENCE TO RELATED APPLICATIONS

This application claims priority to U.S. Provisional Application Ser. No. 63/191,202, filed May 20, 2021, which is incorporated by reference herein in its entirety.

### STATEMENT REGARDING FEDERALLY SPONSORED RESEARCH OR DEVELOPMENT

This invention was made with government support under grant number CA214749 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 16, 2022, is named MDACP1293WO_ST25.txt and is 2,529 bytes in size.

### BACKGROUND

### I. Field of the Disclosure

Aspects of this disclosure relate, generally, to at least the fields of cell biology, molecular biology, cancer biology, and medicine.

### II. Background

Clinical options for treatment of disease and delivery of therapeutic agents are always in demand and in need of improvement. The present disclosure satisfies needs in the art by providing a reliable, reproducible, and practical system for producing exosomes as a means for therapy and/or for therapeutic agent delivery.

### SUMMARY

The present disclosure is directed to systems, methods, and compositions for production and use of exosomes, optionally wherein the exosomes are loaded with one or more therapeutic agents. In particular aspects, the disclosure concerns systems, methods, and compositions for production of exosomes for the purpose of being used as a treatment or as part of a treatment, including as a therapeutic agent or as part of a therapeutic agent for use for an individual in need thereof, and/or as a delivery agent or as part of a delivery agent itself to deliver one or more therapeutic agents to an individual in need thereof. Also disclosed, in some aspects, are methods of treating any medical disorder for which the exosomes, optionally loaded with one or more therapeutic agents, would be therapeutic. In some aspects, the exosomes, optionally loaded with one or more therapeutic agents, can both treat a disease or a condition in an individual and protect against toxicities associated with other treatments for said disease or condition administered to the individual. In certain aspects, exosomes are produced from particular cells using multiple agents in the production method of the exosomes. Such exosomes may be produced from particular cells, including at least stem cells, and for example, mesenchymal stem cells (MSCs, which may also be referred to as mesenchymal stromal cells). The MSCs may be derived from any suitable tissue, but in a specific case they are derived from umbilical cord tissue. Such exosomes may be modified to harbor one or more therapeutic agents, and in some cases, the exosomes are electroporated to be made to harbor one or more therapeutic agents.

Disclosed herein, in some aspects, is a method of producing therapeutic exosomes comprising the steps of. (a) culturing mesenchymal stem cells (MSCs); (b) collecting the exosomes from the culture; and (c) electroporating the collected exosomes to load one or more therapeutic agents into the exosomes. Additionally, or alternatively, in some aspects, prior to step (a), MSCs are transduced or transfected to load one or more therapeutic agents into the MSCs, MSCs are cultured at step (a), and exosomes generated from the transfected or transduced MSCs and comprising the one or more therapeutic agents are collected from culture. In some aspects, the culturing step (a) occurs in the presence of specific concentrations or conditions of CO₂, O₂, and nitrogen. In some aspects, the concentration of CO₂ is 5%. In some aspects, the concentration of O₂ is 20%. In some aspects, the culturing step (a) occurs under conditions balanced with nitrogen.

In some aspects, the MSCs are from umbilical cord tissue, bone marrow, adipose tissue, dental tissue, placental tissue, or a mixture thereof. In some aspects, the MSCs are from umbilical cord tissue.

In some aspects, the method occurs in an automated system. In some aspects, system is configured to comprise continuous perfusion of medium through at least part of the system. In some aspects, the system is closed or semi-closed. In some aspects, the method occurs in a bioreactor. In some aspects, the bioreactor comprises multiple hollow fibers. In some aspects, one or more surfaces inside the bioreactor are modified to allow adherence of cells. In some aspects, the one or more surfaces inside the bioreactor are modified to comprise one or more extracellular matrix proteins. In some aspects, the extracellular matrix protein is fibronectin.

In some aspects, the method further comprises the step of extracting a sample from the system. In some aspects, the sample is tested for one or more characteristics of the exosomes.

In some aspects, step (b) of the method utilizes media that lacks platelet lysate. In some aspects, step (b) of the method utilizes media that comprises L-alanyl-L-glutamine dipeptide. In some aspects, the culturing step (a) of the method utilizes media that comprises L-alanyl-L-glutamine dipeptide. In some aspects, the culturing step (a) of the method utilizes alpha MEM media, heparin, human platelet lysate, and L-alanyl-L-glutamine dipeptide. In some aspects of the method, steps (a) and (b) occur more than once. In some aspects of the method, steps (a) and (b) occur 2, 3, 4, 5, 6, 7, 8, 9, 10, or more times. In some aspects of the method, step (b) occurs more than once and the collecting occurs in intervals of about 48 hours.

In some aspects, the collected exosomes are suspended in a sterile, isotonic, non-pyrogenic buffer prior to electroporation in step (c). In some aspects, the buffer comprises Plasma-Lyte A. In some aspects, about 1×10⁸ to about 10×10¹² collected exosomes are electroporated in step (c).

In some aspects, the one or more therapeutic agents is miRNA, siRNA, shRNA, protein, peptides, drug, lipids, DNA, RNA, or a combination thereof. In some aspects, the one or more therapeutic agents is protein, peptides, drugs, and/or lipids, and wherein the concentration of the protein, peptides, drugs, and/or lipids is between 1 μg/mL and 1000 mg/mL. In some aspects, the protein comprises an antibody or antibody fragment. In some aspects, the one or more therapeutic agents is miRNA, and wherein the concentration of miRNA is between 1 μg/mL and 200 μg/mL. In some aspects, the miRNA comprises miR-124, miR-148a, miR-let7i, miR-135a-2, miR-668, miR-942, miR-657. In some aspects, the one or more therapeutic agents is siRNA, shRNA, and/or RNA, and wherein the concentration of siRNA, shRNA, and/or RNA is between 1 μg/mL and 200 μg/mL. In some aspects, the siRNA comprises siRNA against the fusion breakpoint of a FGFR3-TACC3 gene fusion product. In some aspects, the DNA comprises at most 1000 base pairs. In some aspects, the concentration of DNA is between 1 μg/mL and 200 μg/mL. In some aspects, the loading efficiency of the one or more therapeutic agents is at least 30%, at least 40%, at least 50%, at least 60%, at least 70%, at least 80%, or at least 90%.

In some aspects, the method further comprises the step of delivering an effective amount of the exosomes to an individual in need thereof. In some aspects, following delivery to an individual in need thereof, the exosomes provide neuroprotection against central nervous system toxicities induced by one or more additional therapies delivered to the individual. In some aspects, following delivery to an individual in need thereof, the exosomes ameliorate or reverse cognitive dysfunction and/or neurodegeneration induced by one or more additional therapies delivered to the individual. In some aspects, the one or more additional therapies delivered to the individual comprise chemotherapy, radiotherapy, or a combination thereof. In some aspects, following delivery to an individual in need thereof, the exosomes reduce inflammation.

In some aspects, following delivery to an individual in need thereof, the exosomes provide a tissue-regenerative effect to a tissue in need of regeneration. In some aspects, following delivery to an individual in need thereof, the exosomes provide a tissue-reparative effect to a tissue in need of repair. In some aspects, the tissue is in need of regeneration or repair due to toxicities induced by one or more additional therapies delivered to the individual. In some aspects, the one or more additional therapies delivered to the individual comprise chemotherapy, radiotherapy, or a combination thereof. In some aspects, the tissue is in need of regeneration or repair due to toxicity due to burns (e.g., thermal burns, chemical burns, electric burns, frostbite) or trauma (e.g., contusions, sprains, tendonitis, bursitis, stress injuries, strains) and/or toxicity due to a prior treatment for burns (e.g., thermal burns, chemical burns, electric burns, frostbite) or trauma (e.g., contusions, sprains, tendonitis, bursitis, stress injuries, strains). In some aspects, the tissue is in need of regeneration or repair due to a tissue injury. Non-limiting examples of tissue injuries include inflammation, contusions, sprains, tendonitis, bursitis, stress injuries, strains, or a combination thereof. In some aspects, the tissue in need of regeneration or reparation comprises soft tissue (e.g., fat, fibrous tissue (e.g., tendons and/or ligaments), muscle (e.g., smooth muscle, skeletal muscle, and/or cardiac muscle), synovial tissue, blood vessels, lymph vessels, and/or nerves), brain, lung, spleen, liver, heart, kidney, pancreas, intestine, testis, or bone.

In some aspects, following delivery to an individual in need thereof, the exosomes provide a skin-regenerative effect to skin in need of regeneration. In some aspects, following delivery to an individual in need thereof, the exosomes provide a skin-reparative effect to skin in need of repair. In some aspects, the skin is in need of regeneration or repair due to toxicities induced by one or more additional therapies delivered to the individual. In some aspects, the one or more additional therapies delivered to the individual comprise chemotherapy, radiotherapy, or a combination thereof. In some aspects, the skin is in need of regeneration or repair due to toxicity due to burns (e.g., thermal burns, chemical burns, electric burns, frostbite) or trauma (e.g., cutaneous wound damage, contusions, cuts, lacerations, gashes, tears, punctures, scrapes, abrasions, scratches, bites, stings, bruises, pressure injuries, crush injuries, incisions) and/or toxicity due to a prior treatment for burns (e.g., thermal burns, chemical burns, electric burns, frostbite) or trauma (e.g., cutaneous wound damage, contusions, cuts, lacerations, gashes, tears, punctures, scrapes, abrasions, scratches, bites, stings, bruises, pressure injuries, crush injuries, incisions). In some aspects, the skin is in need of regeneration or repair due to a skin disorder. Non-limiting examples of skin disorders include inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

In some aspects, following delivery to an individual in need thereof, the exosomes provide a wound healing effect (e.g., regenerative repair) to tissue or skin in need of wound healing. In some aspects, the tissue or skin is in need of wound healing (e.g., regenerative repair) due to toxicities induced by one or more additional therapies delivered to the individual. In some aspects, the one or more additional therapies delivered to the individual comprise chemotherapy, radiotherapy, or a combination thereof. In some aspects, the tissue or skin is in need of wound healing (e.g., regenerative repair) due to toxicity due to burns (e.g., thermal burns, chemical burns, electric burns, frostbite) or trauma (e.g., sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions) and/or toxicity due to a prior treatment for burns (e.g., thermal burns, chemical burns, electric burns, frostbite) or trauma (e.g., sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions). In some aspects, the tissue or skin is in need of wound healing (e.g., regenerative repair) due to a tissue injury or a skin disorder. Non-limiting examples of tissue injuries and skin disorders include inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

In some aspects, the exosomes cross the blood-brain barrier, the blood-tumor barrier, or a combination thereof. In some aspects, the exosomes directly or indirectly treat an immune disorder, cancer, heart disease, kidney disease, lung disease, liver disease, infection, inflammation, tissue injury, a skin disorder, wounds, or a combination thereof, or one or more symptoms of an immune disorder, cancer, heart disease, kidney disease, lung disease, liver disease, infection, inflammation, tissue injury, a skin disorder, wounds, trauma, burns, or a combination thereof, in an individual in need thereof having an immune disorder, cancer, heart disease, kidney disease, lung disease, liver disease, infection, inflammation, tissue injury, a skin disorder, wounds, trauma, burns, or a combination thereof. In some aspects, the one or more symptoms comprise central nervous system toxicities, cognitive dysfunction, neurodegeneration, inflammation, tissue degeneration, tissue damage, skin damage, wounds, trauma, burns, or a combination thereof.

In some aspects, the immune disorder is an autoimmune disorder or an alloimmune disorder. In some aspects, the immune disorder is graft-versus-host disease. In some aspects, the cancer is a solid tumor cancer. In some aspects, the cancer is a CNS cancer or a CNS-related cancer. In some aspects, the cancer is glioblastoma. In some aspects, the tissue injury is inflammation, a contusion, a sprain, tendonitis, bursitis, a stress injury, a strain, or a combination thereof. In some aspects, the skin disorder is inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof. In some aspects, the wound is a result of a tissue injury or skin disorder, and the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof. In some aspects, the burns are thermal burns, chemical burns, electric burns, frostbite, or a combination thereof. In some aspects, the trauma is sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions, or a combination thereof.

Disclosed herein, in some aspects, are exosomes produced from any one of the methods disclosed herein; compositions comprising the exosomes; and pharmaceutical compositions comprising the exosomes, wherein the pharmaceutical compositions optionally further comprise one or more additional therapeutic agents.

Disclosed herein, in some aspects, is a method of treating an individual for an immune disorder, cancer, heart disease, kidney disease, lung disease, liver disease, infection, inflammation, tissue injury, a skin disorder, wounds, trauma, burns, or a combination thereof, comprising the step of administering to the individual a therapeutically effective amount of exosomes produced from any one of the methods disclosed herein. In some aspects, the cancer is a solid tumor cancer. In some aspects, the cancer is a CNS cancer or a CNS-related cancer.

In some aspects, the cancer is glioblastoma. In some aspects, the immune disorder is an alloimmune disorder or an autoimmune disorder. In some aspects, the immune disorder is graft-versus-host disease. In some aspects, the tissue injury is inflammation, a contusion, a sprain, tendonitis, bursitis, a stress injury, a strain, or a combination thereof. In some aspects, the skin disorder is inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof. In some aspects, the wound is a result of a tissue injury or skin disorder, and the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof. In some aspects, the burns are thermal burns, chemical burns, electric burns, frostbite, or a combination thereof. In some aspects, the trauma is sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions, or a combination thereof.

Disclosed herein, in some aspects, is a method of protecting against central nervous system toxicities in an individual having an immune disorder, cancer, heart disease, kidney disease, lung disease, liver disease, infection, inflammation, tissue injury, a skin disorder, wounds, trauma, burns, or a combination thereof, wherein the central nervous system toxicities are induced by one or more therapies delivered to the individual for treatment of the immune disorder, cancer, heart disease, kidney disease, lung disease, liver disease, infection, inflammation, tissue injury, a skin disorder, wounds, trauma, burns, or a combination thereof, the method comprising the step of administering to the individual a therapeutically effective amount of exosomes or compositions thereof produced from any one of the methods disclosed herein.

Disclosed herein, in some aspects, is a method of ameliorating or reversing cognitive dysfunction and/or neurodegeneration in an individual having an immune disorder, cancer, heart disease, kidney disease, lung disease, liver disease, infection, inflammation, tissue injury, a skin disorder, wounds, trauma, burns, or a combination thereof, wherein the cognitive dysfunction and/or neurodegeneration is induced by one or more therapies delivered to the individual for treatment of the immune disorder, cancer, heart disease, kidney disease, lung disease, liver disease, infection, inflammation, tissue injury, a skin disorder, wounds, trauma, burns, or a combination thereof, the method comprising the step of administering to the individual a therapeutically effective amount of exosomes or compositions thereof produced from any one of the methods disclosed herein.

Disclosed herein, in some aspects, is a method of regenerating and/or repairing tissue in need thereof in an individual having an immune disorder, cancer, heart disease, kidney disease, lung disease, liver disease, infection, inflammation, tissue injury, a skin disorder, wounds, trauma, burns, or a combination thereof. In some aspects, the tissue is in need of regeneration and/or repair due to toxicities induced by one or more therapies delivered to the individual for treatment of the immune disorder, cancer, heart disease, kidney disease, lung disease, liver disease, infection, inflammation, tissue injury, a skin disorder, wounds, trauma, burns, or a combination thereof. In some aspects, the one or more therapies received by the individual comprise chemotherapy, radiotherapy, or a combination thereof. Disclosed herein, in some aspects, is a method of regenerating and/or repairing tissue in need thereof in an individual having a tissue injury comprising inflammation, contusions, sprains, tendonitis, bursitis, stress injuries, strains, or a combination thereof. In some aspects, the tissue in need of regeneration or reparation comprises soft tissue (e.g., fat, fibrous tissue (e.g., tendons and/or ligaments), muscle (e.g., smooth muscle, skeletal muscle, and/or cardiac muscle), synovial tissue, blood vessels, lymph vessels, and/or nerves), brain, lung, spleen, liver, heart, kidney, pancreas, intestine, testis, or bone. In some aspects, the method comprises the step of administering to the individual a therapeutically effective amount of exosomes or compositions thereof produced from any one of the methods disclosed herein.

Disclosed herein, in some aspects, is a method of regenerating and/or repairing skin in need thereof in an individual having an immune disorder, cancer, heart disease, kidney disease, lung disease, liver disease, infection, inflammation, tissue injury, a skin disorder, wounds, trauma, burns, or a combination thereof. In some aspects, the skin is in need of regeneration and/or repair due to toxicities induced by one or more therapies delivered to the individual for treatment of the immune disorder, cancer, heart disease, kidney disease, lung disease, liver disease, infection, inflammation, tissue injury, a skin disorder, wounds, trauma, burns, or a combination thereof. In some aspects, the one or more therapies received by the individual comprise chemotherapy, radiotherapy, or a combination thereof. Disclosed herein, in some aspects, is a method of regenerating and/or repairing skin in need thereof in an individual having a skin disorder comprising inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof. In some aspects, the method comprises the step of administering to the individual a therapeutically effective amount of exosomes or compositions thereof produced from any one of the methods disclosed herein.

Disclosed herein, in some aspects, is a method of wound healing (e.g., wound repair) in tissue or skin in need thereof in an individual having an immune disorder, cancer, heart disease, kidney disease, lung disease, liver disease, infection, inflammation, tissue injury, a skin disorder, wounds, trauma, burns, or a combination thereof. In some aspects, the tissue or skin is in need of wound healing (e.g., wound repair) due to toxicities induced by one or more therapies delivered to the individual for treatment of the immune disorder, cancer, heart disease, kidney disease, lung disease, liver disease, infection, inflammation, tissue injury, a skin disorder, wounds, trauma, burns, or a combination thereof. In some aspects, the one or more therapies received by the individual comprise chemotherapy, radiotherapy, or a combination thereof. Disclosed herein, in some aspects, is a method of wound healing (e.g., wound repair) in tissue or skin in need thereof in an individual having a tissue injury or skin disorder comprising inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof. In some aspects, the method comprises the step of administering to the individual a therapeutically effective amount of exosomes or compositions thereof produced from any one of the methods disclosed herein.

Disclosed herein, in some aspects, is a method of reducing inflammation in an individual having an immune disorder, cancer, heart disease, kidney disease, lung disease, liver disease, infection, inflammation, tissue injury, a skin disorder, wounds, trauma, burns, or a combination thereof, comprising the step of administering to the individual a therapeutically effective amount of exosomes or compositions thereof produced from any one of the methods disclosed herein.

In some aspects of the methods of treating an individual disclosed herein, the exosomes cross the blood-brain barrier, the blood-tumor barrier, or a combination thereof. In some aspects, the MSCs are autologous or allogeneic with respect to the individual. In some aspects, the exosomes are administered via rectal, nasal, buccal, vaginal, subcutaneous, intranasal, intracutaneous, intravenous, intraperitoneal, intramuscular, intraarticular, intrasynovial, intrasternal, intrathecal, intralesional, or intracranial routes, or via an implanted reservoir. In some aspects, the exosomes are administered in conjunction with at least one additional therapeutic agent.

Also disclosed are the following Aspects (A) 1 to 98 of the present disclosure.

Aspect 1 is a method of producing therapeutic exosomes comprising the steps of: (a) culturing mesenchymal stem cells (MSCs); and (b) collecting the exosomes from the culture; wherein, prior to step (a), the MSCs are transfected or transduced to load one or more therapeutic agents into the MSCs, and wherein the exosomes are generated from the transfected or transduced MSCs and comprise the one or more therapeutic agents.

Aspect 2 is a method of producing therapeutic exosomes comprising the steps of: (a) culturing mesenchymal stem cells (MSCs); (b) collecting the exosomes from the culture; and (c) electroporating the collected exosomes to load one or more therapeutic agents into the exosomes.

A3. The method of A1 or A2, wherein the culturing step (a) occurs in the presence of specific concentrations or conditions of CO₂, O₂, and nitrogen.

A4. The method of A3, wherein the concentration of CO₂ is 5%.

A5. The method of A3 or A4, wherein the concentration of O₂ is 20%.

A6. The method of A1 to A5, wherein the culturing step (a) occurs under conditions balanced with nitrogen.

A7. The method of A1 to A6, wherein the MSCs are from umbilical cord tissue, bone marrow, adipose tissue, dental tissue, placental tissue, or a mixture thereof.

A8. The method of A1 to A7, wherein the MSCs are from umbilical cord tissue.

A9. The method of A1 to A8, wherein the method occurs in an automated system.

A10. The method of A9, wherein system is configured to comprise continuous perfusion of medium through at least part of the system.

A11. The method of A9 or A10, wherein the system is closed or semi-closed.

A12. The method of A1 to A11, wherein the method occurs in a bioreactor.

A13. The method of A12, wherein the bioreactor comprises multiple hollow fibers.

A14. The method of A13, wherein one or more surfaces inside the bioreactor are modified to allow adherence of cells.

A15. The method of A14, wherein the one or more surfaces inside the bioreactor are modified to comprise one or more extracellular matrix proteins.

A16. The method of A15, wherein the extracellular matrix protein is fibronectin.

A17. The method of A9 to A16, further comprising the step of extracting a sample from the system.

A18. The method of A17, wherein the sample is tested for one or more characteristics of the exosomes.

A19. The method of A1 to A18, wherein step (b) utilizes media that lacks platelet lysate.

A20. The method of A1 to A19, wherein step (b) utilizes media that comprises L-alanyl-L-glutamine dipeptide.

A21. The method of A1 to A20, wherein the culturing step (a) utilizes media that comprises L-alanyl-L-glutamine dipeptide.

A22. The method of A1 to A21, wherein the culturing step (a) utilizes alpha MEM media, heparin, human platelet lysate, and L-alanyl-L-glutamine dipeptide.

A23. The method of A1 to A22, wherein steps (a) and (b) occur more than once.

A24. The method of A1 to A23, wherein steps (a) and (b) occur 2, 3, 4, 5, 6, 7, 8, 9, 10, or more times.

A25. The method of A1 to A24, wherein step (b) occurs more than once and the collecting occurs in intervals of about 48 hours.

A26. The method of A2 to A25, wherein the collected exosomes are suspended in a sterile, isotonic, non-pyrogenic buffer prior to electroporation in step (c).

A27. The method A26, wherein the buffer comprises Plasma-Lyte A.

A28. The method of A2 to A27, wherein about 1×10⁸ to about 10×10¹² collected exosomes are electroporated in step (c).

A29. The method of A1 to A28, wherein the one or more therapeutic agents is miRNA, siRNA, shRNA, protein, peptides, drug, lipids, DNA, RNA, or a combination thereof.

A30. The method of A29, wherein the one or more therapeutic agents is protein, peptides, drugs, and/or lipids, and wherein the concentration of the protein, peptides, drugs, and/or lipids is between 1 μg/mL and 1000 mg/mL.

A31. The method of A29 or A30, wherein the protein comprises an antibody or antibody fragment.

A32. The method of A29 to A31, wherein the one or more therapeutic agents is miRNA, and wherein the concentration of miRNA is between 1 μg/mL and 200 μg/mL.

A33. The method of A29 to A32, wherein the miRNA comprises miR-124, miR-148a, miR-let7i, miR-135a-2, miR-668, miR-942, miR-657.

A34. The method A29 to A33, wherein the one or more therapeutic agents is siRNA, shRNA, and/or RNA, and wherein the concentration of siRNA, shRNA, and/or RNA is between 1 μg/mL and 200 μg/mL.

A35. The method of A29 to A34, wherein the siRNA comprises siRNA against the fusion breakpoint of a FGFR3-TACC3 gene fusion product.

A36. The method of A29 to A35, wherein the DNA comprises at most 1000 base pairs.

A37. The method of A29 to A36, wherein the concentration of DNA is between 1 μg/mL and 200 μg/mL.

A38. The method of A1 to A37, wherein the loading efficiency of the one or more therapeutic agents is at least 30%, at least 40%, at least 50%, at least 60%, at least 70%, at least 80%, or at least 90%.

A39. The method of A1 to A38, further comprising the step of delivering an effective amount of the exosomes to an individual in need thereof.

A40. The method of A39, wherein following delivery to an individual in need thereof, the exosomes provide neuroprotection against central nervous system toxicities induced by one or more additional therapies delivered to the individual.

A41. The method of A39 or A40, wherein following delivery to an individual in need thereof, the exosomes ameliorate or reverse cognitive dysfunction and/or neurodegeneration induced by one or more additional therapies delivered to the individual.

A42. The method of A40 or A41, wherein the one or more additional therapies delivered to the individual comprise chemotherapy, radiotherapy, or a combination thereof.

A43. The method of A39 to A42, wherein following delivery to an individual in need thereof, the exosomes reduce inflammation.

A44. The method of A39 to A43, wherein following delivery to an individual in need thereof, the exosomes provide a tissue-regenerative and/or a tissue-reparative effect to a tissue in need of regeneration and/or repair.

A45. The method of A44, wherein the tissue is in need of regeneration and/or repair due to toxicities induced by one or more additional therapies delivered to the individual.

A46. The method of A44 or A45, wherein the one or more additional therapies delivered to the individual comprise chemotherapy, radiotherapy, or a combination thereof.

A47. The method of A44 to A46, wherein the tissue is in need of regeneration and/or repair due to inflammation, contusions, sprains, tendonitis, bursitis, stress injuries, strains, or a combination thereof.

A48. The method of A44 to A47, wherein the tissue in need of regeneration and/or repair comprises soft tissue, brain, lung, spleen, liver, heart, kidney, pancreas, intestine, testis, or bone.

A49. The method of A39 to A48, wherein following delivery to an individual in need thereof, the exosomes provide a skin-regenerative and/or a skin-reparative effect to skin in need of regeneration and/or repair.

A50. The method of A49, wherein the skin is in need of regeneration and/or repair due to toxicities induced by one or more additional therapies delivered to the individual.

A51. The method of A49 or A50, wherein the one or more additional therapies delivered to the individual comprise chemotherapy, radiotherapy, or a combination thereof.

A52. The method of A49 to A51, wherein the skin is in need of regeneration and/or repair due to a skin disorder.

A53. The method of A49 to A52, wherein the skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

A54. The method of A39 to A53, wherein following delivery to an individual in need thereof, the exosomes provide a wound healing effect to tissue or skin in need of wound healing.

A55. The method of A54, wherein the tissue or skin is in need of wound healing due to toxicities induced by one or more additional therapies delivered to the individual.

A56. The method of A54 or A55, wherein the one or more additional therapies delivered to the individual comprise chemotherapy, radiotherapy, or a combination thereof.

A57. The method of A54 to A56, wherein the tissue or skin is in need of wound healing due to a tissue injury or skin disorder.

A58. The method of A54 to A57, wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

A59. The method of A39 to A58, wherein the exosomes cross the blood-brain barrier, the blood-tumor barrier, or a combination thereof.

A60. The method of A39 to A59, wherein the exosomes directly or indirectly treat an immune disorder, cancer, heart disease, kidney disease, lung disease, liver disease, infection, tissue injury, skin disorder, wound, or a combination thereof, or one or more symptoms of an immune disorder, cancer, heart disease, kidney disease, lung disease, liver disease, infection, tissue injury, skin disorder, wound, or a combination thereof, in an individual in need thereof having an immune disorder, cancer, heart disease, kidney disease, lung disease, liver disease, infection, tissue injury, skin disorder, wound, or a combination thereof.

A61. The method of A60, wherein the one or more symptoms comprise central nervous system toxicities, cognitive dysfunction, neurodegeneration, inflammation, tissue degeneration, tissue damage, skin damage, wounds, or a combination thereof.

A62. The method of A60 or A61, wherein the immune disorder is an autoimmune disorder or an alloimmune disorder.

A63. The method of A62, wherein the immune disorder is graft-versus-host disease.

A64. The method of A60 or A61, wherein the cancer is a solid tumor cancer.

A65. The method of A64, wherein the cancer is a CNS cancer or a CNS-related cancer.

A66. The method of A64 or A65, wherein the cancer is glioblastoma.

A67. The method of A60 or A61, wherein the tissue injury is inflammation, contusions, sprains, tendonitis, bursitis, stress injuries, strains, or a combination thereof.

A68. The method of A60 or A61, wherein the skin disorder is inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

A69. The method of A60 or A61, wherein the wound is a result of a tissue injury or skin disorder, and wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

Aspect 70 are exosomes produced from any one of the methods of A1 to A37.

Aspect 71 is a composition comprising the exosomes of A70.

Aspect 72 is a pharmaceutical composition comprising the exosomes of A70.

A73. The pharmaceutical composition of A72, further comprising one or more additional therapeutic agents.

Aspect 74 is a method of treating an individual for an immune disorder, cancer, heart disease, kidney disease, lung disease, liver disease, infection, tissue injury, skin disorder, wound, or a combination thereof, comprising the step of administering to the individual a therapeutically effective amount of exosomes produced by the method of A1 to A37, the composition of A71, or the pharmaceutical composition of A72 to A73.

A75. The method of A74, wherein the cancer is a solid tumor cancer.

A76. The method of A75, wherein the cancer is a CNS cancer or a CNS-related cancer.

A77. The method of A75 or A76, wherein the cancer is glioblastoma.

A78. The method of A74, wherein the immune disorder is an alloimmune disorder or an autoimmune disorder.

A79. The method of A78, wherein the immune disorder is graft-versus-host disease.

A80. The method of A74, wherein the tissue injury is inflammation, contusions, sprains, tendonitis, bursitis, stress injuries, strains, or a combination thereof.

A81. The method of A74, wherein the skin disorder is inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

A82. The method of A74, wherein the wound is a result of a tissue injury or skin disorder, and wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

A83. The method of A74 to A82, further comprising administering to the individual a second therapy for the respective immune disorder, cancer, heart disease, kidney disease, lung disease, liver disease, infection, tissue injury, skin disorder, wound, or a combination thereof.

Aspect 84 is a method of protecting against central nervous system toxicities in an individual having an immune disorder, cancer, heart disease, kidney disease, lung disease, liver disease, infection, tissue injury, skin disorder, wound, or a combination thereof, wherein the central nervous system toxicities are induced by one or more therapies delivered to the individual for treatment of the immune disorder, cancer, heart disease, kidney disease, lung disease, liver disease, infection, or a combination thereof, the method comprising the step of administering to the individual a therapeutically effective amount of exosomes produced by the method of A1 to A37, the composition of A71, or the pharmaceutical composition of A72 to A73.

Aspect 85 is a method of ameliorating or reversing cognitive dysfunction and/or neurodegeneration in an individual having an immune disorder, cancer, heart disease, kidney disease, lung disease, liver disease, infection, tissue injury, skin disorder, wound, or a combination thereof, wherein the cognitive dysfunction and/or neurodegeneration is induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of A1 to A37, the composition of A71, or the pharmaceutical composition of A72 to A73.

Aspect 86 is a method of regenerating and/or repairing tissue in need thereof in an individual having an immune disorder, cancer, heart disease, kidney disease, lung disease, liver disease, infection, tissue injury, skin disorder, wound, or a combination thereof, wherein the tissue is in need of regeneration and/or reparation due to toxicities induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of A1 to A37, the composition of A71, or the pharmaceutical composition of A72 to A73.

Aspect 87 is a method of regenerating and/or repairing skin in need thereof in an individual having an immune disorder, cancer, heart disease, kidney disease, lung disease, liver disease, infection, tissue injury, skin disorder, wound, or a combination thereof, wherein the skin is in need of regeneration and/or reparation due to toxicities induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of A1 to A37, the composition of A71, or the pharmaceutical composition of A72 to A73.

Aspect 88 is a method of wound healing in a tissue or skin in need thereof in an individual having an immune disorder, cancer, heart disease, kidney disease, lung disease, liver disease, infection, tissue injury, skin disorder, wound, or a combination thereof, wherein the tissue or skin is in need of wound healing due to toxicities induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of A1 to A37, the composition of A71, or the pharmaceutical composition of A72 to A73.

A89. The method of A84 to A88, wherein the one or more therapies received by the individual comprise chemotherapy, radiotherapy, or a combination thereof.

Aspect 90 is a method of regenerating and/or repairing tissue in need thereof in an individual having a tissue injury, wherein the tissue injury comprises inflammation, contusions, sprains, tendonitis, bursitis, stress injuries, strains, or a combination thereof, the method comprising the step of administering to the individual a therapeutically effective amount of exosomes produced by the method of A1 to A37, the composition of A71, or the pharmaceutical composition of A72 to A73.

A91. The method of A86 to A90, wherein the tissue in need of regeneration and/or reparation comprises soft tissue, brain, lung, spleen, liver, heart, kidney, pancreas, intestine, testis, or bone.

Aspect 92 is a method of regenerating and/or repairing skin in need thereof in an individual having a skin disorder, wherein the skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof, the method comprising the step of administering to the individual a therapeutically effective amount of exosomes produced by the method of A1 to A37, the composition of A71, or the pharmaceutical composition of A72 to A73.

Aspect 93 is a method of wound healing in tissue or skin in need thereof in an individual having a tissue injury or skin disorder, wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof, the method comprising the step of administering to the individual a therapeutically effective amount of exosomes produced by the method of A1 to A37, the composition of A71, or the pharmaceutical composition of A72 to A73.

Aspect 94 is a method of reducing inflammation in an individual having an immune disorder, cancer, heart disease, kidney disease, lung disease, liver disease, infection, or a combination thereof, comprising the step of administering to the individual a therapeutically effective amount of exosomes produced by the method of A1 to A37, the composition of A71, or the pharmaceutical composition of A72 to A73.

A95. The method of A74 to A94, wherein the exosomes cross the blood-brain barrier, the blood-tumor barrier, or a combination thereof.

A96. The method of A74 to A95, wherein the MSCs are autologous or allogeneic with respect to the individual.

A97. The method A74 to A96, wherein the exosomes are administered via rectal, buccal, vaginal, subcutaneous, intranasal, intracutaneous, intravenous, intraperitoneal, intramuscular, intraarticular, intrasynovial, intrasternal, intrathecal, intralesional, or intracranial routes, or via an implanted reservoir.

A98. The method of A74 to A97, wherein the exosomes are administered in conjunction with at least one additional therapeutic agent.

The term “therapeutically effective amount” refers to an amount sufficient to produce a desired therapeutic result, for example an amount of exosomes sufficient to improve at least one symptom of a medical condition in a subject to whom the cells are administered.

“Individual, “subject,” and “patient” are used interchangeably and can refer to either a human or non-human, such as primates, mammals, and vertebrates. In particular aspects, the subject is a human. The subject is of any age, gender, or race. The subject can be a patient, e.g., have or be suspected of having a disease (that may be referred to as a medical condition), such as benign or malignant cancer, an auto- or allo-immune condition, an infectious disease, a tissue injury, a skin disorder, or a wound. The subject may be undergoing or have undergone treatment. The subject may be asymptomatic. The subject may be a healthy individual desirous of prevention of a disease or condition.

As used herein, “treat,” “treating,” or “treatment” or equivalent terminology refer to both therapeutic treatment and prophylactic or preventative measures, wherein the object is to prevent or slow down (lessen) an undesired physiological change or disorder, such as the growth, development, or spread of a tumor or infectious disease, tumor relapse, a manifestation of an auto- or allo-immune disorder, a manifestation of a tissue injury, a manifestation of a skin disorder, or a wound. For purposes of this disclosure, beneficial or desired clinical results include, but are not limited to, alleviation or amelioration of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” does not necessarily indicate complete eradication or cure of the disease or condition, or associated symptoms thereof. Those in need of treatment include those already with the condition or disorder as well as those prone to have the condition or disorder or those in which the condition or disorder is to be prevented. The results of treatment can be determined by methods known in the art, such as determination of reduction of, e.g., tumor burden or viral load, determination of restoration of function, or other methods known in the art.

As used herein, “prevent,” and similar words such as “prevented,” “preventing,” etc., indicate an approach for preventing, inhibiting, or reducing the likelihood of the occurrence or recurrence of, a disease or condition, e.g., cancer, an infectious disease, an auto- or allo-immune disorder, a tissue injury, a skin disorder, or a wound. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may include but is not limited to a reduction in the size of a tumor, a reduction in the invasiveness of a tumor, reduction in the growth rate of the cancer, or prevention of metastasis. Treatment of cancer may also refer to prolonging survival of a subject with cancer. Treatment of an infectious disease may include but is not limited to a reduction in the spread of an infectious disease in an individual, a reduction in the invasiveness of an infectious disease, reduction in the rate of transmission of an infectious disease, or prevention of spread of an infectious disease. Treatment of infectious disease may also refer to prolonging survival of a subject with an infectious disease. Treatment of an auto- or allo-immune disorder may include but is not limited to a reduction in pain, edema, elevated temperature, and/or inflammation or prevention of immune rejection. Treatment of an auto- or allo-immune disorder may also refer to prolonging survival of a subject with an auto- or allo-immune disorder. Treatment of a tissue injury may include but is not limited to a reduction in the spread of a tissue injury in an individual or a reduction in the pain or inflammation associated with a tissue injury. Treatment of a tissue injury may also refer to prolonging survival of a subject with a tissue injury. Treatment of a skin disorder may include but is not limited to a reduction in the spread of a skin disorder in an individual or a reduction in the invasiveness of a skin disorder. Treatment of a skin disorder may also refer to prolonging survival of a subject with a skin disorder. Treatment of a wound may include but is not limited to a reduction in the spread of a wound in an individual, a reduction in the invasiveness of a wound, or prevention of infection of a wound. Treatment of a wound may also refer to prolonging survival of a subject with a wound.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

Throughout this application, the terms “about,” “substantially,” and “approximately” are used to indicate that a value includes the inherent variation of error for the measurement or quantitation method or degree of variability in a value or range. Thus, the terms “about,” “substantially,” and “approximately” mean, in general, the stated value plus or minus 5%.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that aspects described herein in the context of the term “comprising” may also be implemented in the context of the term “consisting of” or “consisting essentially of.”

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

It is specifically contemplated that any limitation discussed with respect to one aspect of the disclosure may apply to any other aspect of the disclosure. Furthermore, any composition of the disclosure may be used in any method of the disclosure, and any method of the disclosure may be used to produce or to utilize any composition of the disclosure. Aspects of an embodiment set forth in the Examples are also aspects that may be implemented in the context of aspects discussed elsewhere in a different Example or elsewhere in the application, such as in the Summary, Detailed Description, Claims, and Description of the Drawings.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIG. 1 provides a schematic for the identification of effective anti-glioma miRNAs (miRs).

FIGS. 2A-2B demonstrate that miR-124a is an effective antiglioma miR.

FIG. 3 provides a schematic for the lentiviral engineering of BM-hMSCs to produce exosomes containing miR-124a.

FIGS. 4A-4C show that human bone marrow mesenchymal stem cell (BM-hMSC)-derived vesicles are exosomes.

FIG. 5 shows that BM-hMSC-derived exosomes contain miR-124a (Exo-miR124).

FIG. 6 provides a schematic for assaying the in vitro efficacy of miR-124a-containing exosomes derived from BM-hMSCs.

FIGS. 7A-7B show that BM-hMSC-derived exosomes containing miR-124a (Exo-miR124) inhibit glioma stem cell (GSC) viability (FIG. 7A) and clonogenicity (FIG. 7B).

FIGS. 8A-8C show potential mechanisms of action of BM-hMSC-derived exosomes containing miR-124a (Exo-miR124) characterized by the downregulation FOXA2 (FIG. 8A), lipid accumulation (FIG. 8B), and induction of apoptotic cell death (FIG. 8C) after treatment of GSC267 and GSC8-11 with Exo-miR124.

FIGS. 9A-9E illustrate the identification of miRNAs with inhibitory activity against a panel of seven fully annotated patient-derived patient derived GSCs.

FIG. 10 provides a schematic for assaying the in vivo homing to tumors of miR-124a-containing exosomes (Exos-miR124).

FIGS. 11A-11B illustrate the in vivo homing to tumors of miR-124a-containing exosomes administered by different routes.

FIGS. 12A-12B show that systemic delivery of miR-124a-containing exosomes (Exos-miR124) increases survival of brain tumor-bearing mice in vivo.

FIG. 13 shows that the number of exosomes produced per cell over 24 hours is greater in umbilical cord mesenchymal stem cells (UC-MSCs) than bone marrow mesenchymal stem cells (BM-MSCs).

FIGS. 14A-14B show that UC-MSC-derived exosomes (UC-Exos), like BM-MSC-derived exosomes (BM-Exos), home to brain tumors in vivo. FIG. 14A. BLI of UC-MSC-Exos and BM-MSC-Exos following IA delivery to glioma-bearing mice. FIG. 14B. Bar graph showing average radiant efficiency of brain images, which is calculated using the following formula: (p/s/cm²/sr)/(μW/cm²), where p is photons; s is seconds; sr is steradian; and μW is excitation slit.

FIGS. 15A-15C illustrate the process of producing (FIG. 15A), electroporating (FIG. 15B), and evaluating (FIG. 15C) mesenchymal stem cell (MSC)-derived exosomes loaded with mir-124.

FIG. 16 shows RT-qPCR results assaying miR-124 levels after electroporation via various programs. miRNA concentrations from 0 ng to 1 ug were used to obtain a standard curve (right half of graph).

FIGS. 17A-17D show the effects of treatment with UC-Exos alone (Exosome alone), electroporated UC-Exos containing control miRNA (miR-CTRL), and electroporated UC-Exos containing miR124a (miR-124) on the viability of GSC267 (FIGS. 17A, 17C) and GSC8-11 (FIGS. 17B, 17D) cell lines using a colorimetric assay (FIGS. 17A, 17B) or by counting viable cells using trypan blue exclusion (FIGS. 17C, 17D). *P<0.05, **P<0.01.

FIGS. 18A-18C show the design of siRNA to the F3-T3 fusion breakpoint.

FIG. 18A. Sequence of F3-T3 breakpoint and various custom siRNA constructs. Arrow points to construct #5, which was shown to be most effective in depleting F3-T3 protein in both U87.F3T3 (FIG. 18B) and SNB19.F3T3 (FIG. 18C) cell lines, as illustrated by immunoblotting.

FIG. 19 shows that UC-Exos loaded with iF3T3 successfully deplete F3-T3 in vitro. qPCR assay illustrating that F3-T3 is depleted upon treatment with exosomes loaded with iF3T3 upon electroporation with electroporation programs 1-4. ***P<0.0001, unpaired t-test.

FIG. 20 shows executive function of C57/BL6 mice (n=8/group) treated with chemoradiation followed by treatment with UC-Exos (Exo1: 0.8×10⁹, Exo2: 8.8×10⁹, or Exo3: 1.7×10¹⁰ per mouse IV). Mean time needed to reach the dark compartment in the hard trials is depicted. * P<0.05.

FIGS. 21A-21C describe one example of a procedure designed to produce extracellular vesicles (EVs), such as exosomes, from MSCs using a bioreactor, such as the QUANTUM® Cell Expansion System (Terumo BCT; Lakewood, CO).

FIG. 22 shows the effects of treatment with BM-MSC-Exosomes (BM-MSC-Exos) and two samples of UC-MSC-Exosomes (UC-MSC-Exos) comparing exosomes alone (Empty Exosomes), MSC-Exos containing control miRNA (ExomiR control), and MSC-Exos containing miR124a (miR-124) via lentivirus (BM-MSC-Exos) or electroporation (UC-MSC-Exos) on the viability of the GSC267 glioblastoma cell line assayed by counting viable cells using trypan blue exclusion. The results show that the treatment with exomiR-124 reduced significantly reduced the number glioblastoma stem cells.

FIG. 23 shows RT-qPCR results assaying miR-124 levels on BM-MSC-Exosomes produced by lentivirus and UC-MSC-Exosomes after electroporation using a small or large scale electroporation process, with the appropriate controls. miRNA concentrations from 25ng to 200ng were used to obtain a standard curve (right half of graph). The cord tissue exosomes had the best results in terms of the lower delta Ct indicating a higher copy number of miR124 in the exosome.

### DETAILED DESCRIPTION

The present disclosure describes specific systems, methods, and compositions for producing exosomes from mesenchymal stem cells (MSCs) and for loading said exosomes with therapeutic agents. As demonstrated herein, MSC-derived exosomes prepared according to the disclosed procedures are stable and bioactive, and electroporation of these MSC-derived exosomes can be used to produce exosomes loaded with potent therapeutics.

Umbilical cord (UC)-derived mesenchymal stem cells (UC-MSCs) produce significantly higher numbers of exosomes compared with bone marrow (BM)-derived MSCs (BM-MSCs), in at least some aspects. Additionally, like BM-MSC-derived exosomes, UC-MSC-derived exosomes (UC-Exos) home to tumors in vivo, in at least some aspects, and phenotypic characterization confirmed that UC-MSC and BM-MSC-derived exosomes express the same levels of the exosome markers CD9, CD63, CD47 and CD81. Using electroporation, these exosomes can be directly loaded with therapeutic agents, including but not limited to proteins, nucleic acids and small molecular drugs. This strategy is advantageous because large volume production is more feasible than methods involving transduction of MSCs with lentivirus (LV) to express some therapeutic agents, and the reproducibility of the amount of therapeutic in each exosome is highly controlled, rendering this method more scalable and more “drug-like” than the LV-transduction method, in at least some aspects. Additionally, this strategy separates the production/isolation of the exosomes from the loading with therapeutic agent, allowing for quality control of each stage. Furthermore, in at least some aspects, UC-Exos may be effective at reversing cognitive dysfunction upon chemoradiation induced brain injury and may be effective in the treatment of neurocognitive toxicities secondary to radiation and chemotherapy.

The established approach is practical, efficient, and allows for the clinical use of exosomes as therapeutic agents for, as examples: the treatment of patients with GBM or other solid and liquid tumors, as well as toxicities associated therewith; the treatment of patients with alloimmune or autoimmune disorders; the treatment of patients with microbial infections; the treatment of patients with skin disorders; the treatment of wounds; vehicles for gene and drug delivery; and therapeutic agents for regenerative and/or reparative medicine settings.

### I. Systems and Methods of Producing Exosomes

The present disclosure provides systems and methods of producing extracellular vesicles (EVs) as exosomes. In specific cases, the present disclosure concerns a novel, good manufacturing practice (GMP)-compliant strategy to produce as exosomes. In some aspects, the exosomes are produced under particular conditions in combination with being produced from particular cells.

In particular aspects, the MSCs are from umbilical cord tissue, but they can come from any source including, but not limited to, bone marrow, adipose tissue, dental, and placental tissue.

Any step in the process may have a particular media, duration of time, presence of one or more particular gases at specific concentrations, presence or absence of movement (such as rotation), and a combination thereof, for example. In particular aspects, the cells are incubated (e.g., in a bioreactor or flask) with media for a particular amount of time, in some cases. This is followed by washing and collection of the cells and exosomes secreted from the cells. The collection of the exosomes (that may be referred to herein as harvesting) may include one step or multiple steps; in cases when the collection of the exosomes occurs more than once, there may or may not be an interval of time by which the exosomes are collected, such as at least, at most, or about 12, 18, 24, 36, 48, 60, 72 hours, or more, or any range or value derivable therein, between collections. The media in which the cells and exosomes are collected may be of a particular kind, and in specific steps when the cells and exosomes are collected the media lacks platelet lysate (PLT-free). In specific cases, the cells are cultured over the course of about 22 hours, and then cells are washed and exosomes secreted from the cells are collected approximately every 48 hours in the EC media-PLT-free (the EC media-PLT free may or may not comprise alpha MEM media supplemented with 2 mM of GLUTAMAX™ (synthetic reagent similar to L-glutamine and that comprises L-alanyl-L-glutamine dipeptide)). These sequential steps may be repeated, such as repeated for a total of 2, 3, 4, or more times.

In specific aspects, the suspension of cells and exosomes are harvested from the system under conditions in which the exosomes produced from the cells consistently have the same or substantially the same markers and physiology. Thus, in specific cases at different times of harvesting, the exosomes are the same or substantially the same by their majority of exosomes having one or more of the same expression markers.

In particular aspects, the process to produce the exosomes occurs in a bioreactor, although in alternative cases it does not. In particular aspects, the process to produce the exosomes occurs in flasks. In specific aspects, part or all of the process occurs in a bioreactor having controllable conditions that in specific cases may be automated. Although the bioreactor may be of any kind, in specific aspects the bioreactor comprises a hollow fiber system that may or may not comprise one or more pathways. The multiple hollow fibers comprise inner surfaces suitable for adherence of cells or suitable for modification such that cells may adhere to them, in particular aspects. Alternative systems utilize the WAVE BIOREACTOR™ (GE Healthcare) or the G-REX® system (Wilson Wolf), as examples.

In certain aspects, the hollow fiber bioreactor may be a functionally closed (or semi-closed) system designed for a large-scale cell culture of adherent or non-adherent cells. The system allows the cells to grow (expand in number) in a dynamic environment allowing the continuous perfusion of medium that under suitable conditions mimics particular in vivo intravascular and extravascular compartments in at least some bioreactors. That is, in specific cases an intravascular compartment is configured to mimic the intravascular region of the blood system and/or an extravascular compartment is configured to mimic the extravascular hematopoietic system. The hollow fiber system in specific cases comprises hundreds or thousands of semi-permeable pores for the culture of desired cells, including adherent cells. Membranes may make up the inner walls of the hollow fibers and allow exchange of gas and/or nutrients with a homogenous approach, maximizing the growth rate of the cells in a short time. In particular aspects, the process is specifically designed to be suitable for growth of MSCs and to allow for the collection of the exosomes secreted by the cells in a customized method.

Components of the bioreactor system comprise vessels and/or compartments for introducing media and/or cells to the system, vessels and/or compartments for expanding the cells (and thereby produce exosomes from the expanding/expanded cells), and vessels and/or compartments for harvesting the cells, the conditioned media comprising the exosomes, and so forth. Examples of compartments for any part of the system include a cell inlet bag, media bag, harvest bag, and waste bag, in specific aspects. The bioreactor system utilizes thousands of semi-permeable hollow fibers onto which the cells are adherent, either naturally or because the hollow fibers in the system have been manipulated to allow for adherence of the desired cells. In specific aspects, the system also comprises a gas regulator (that may be referred to as a gas transfer module) that stabilizes desired gas concentrations in the media. Such a gas regulator allows for, if desired, continual infusion of one or more gases into the bioreactor. In specific aspects, the process to produce the desired exosomes utilizes well-defined concentrations of CO₂ (for example, about 5%), O₂ (for example, about 20%), and nitrogen (for example, the conditions are nitrogen balanced).

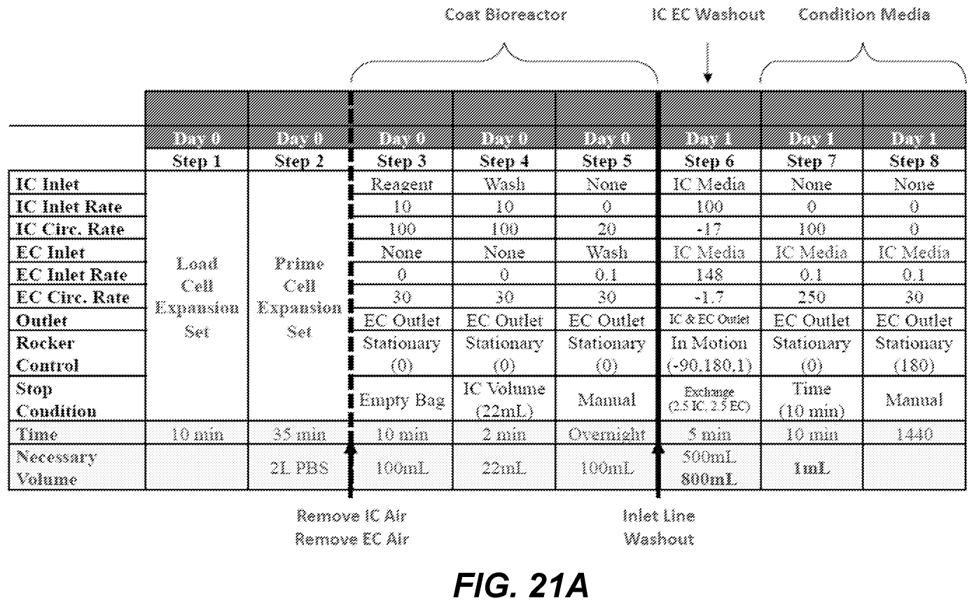

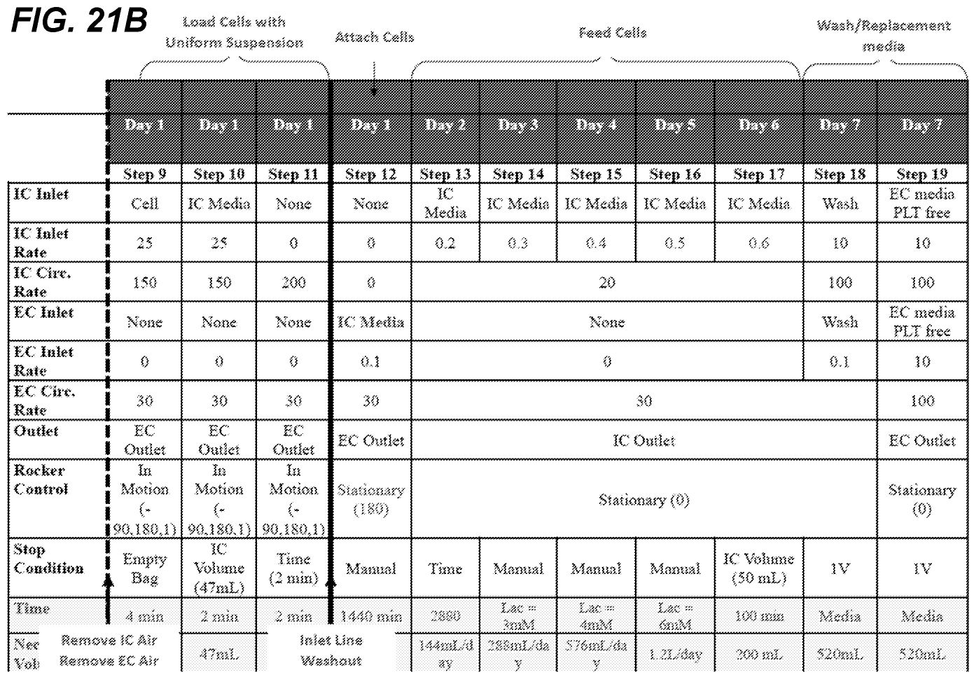

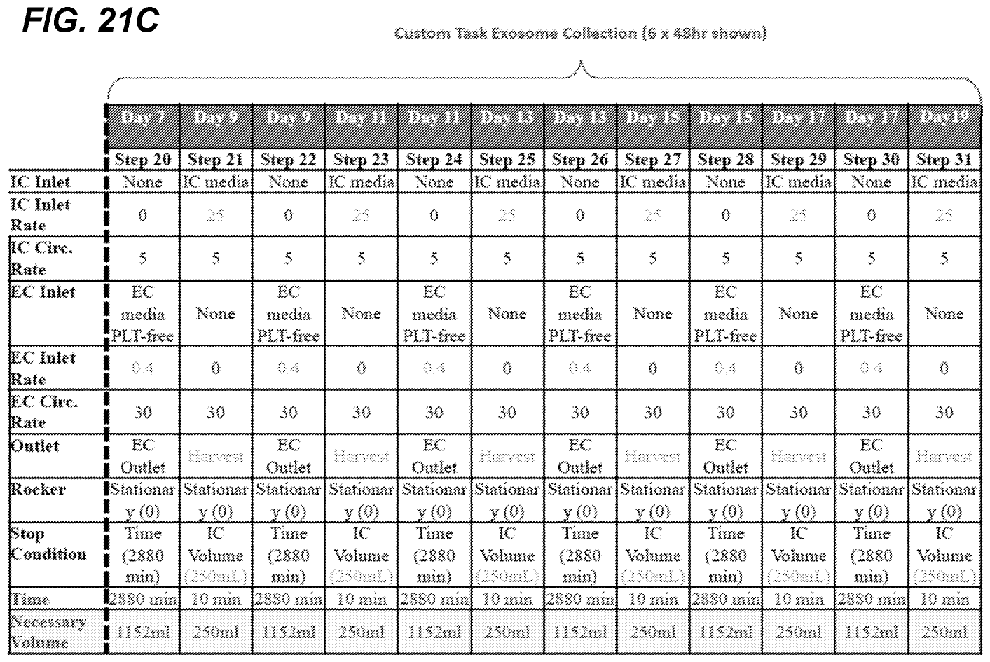

FIGS. 21A-21C provide one example of a procedure and system for producing exosomes. In specific cases of the system, there may be an intracapillary (IC) pathway and/or an extracapillary (EC) pathway. In cases wherein the bioreactor comprises an IC and/or EC pathway, they may be maintained by inlet pumps that determine the flow of new medium into each side of the bioreactor, and circulation pumps that determine the rate at which the medium in each side of the bioreactor is moved through its circuit. In particular aspects for the bioreactor system, a hollow fiber bioreactor system is utilized that is formed by micropores and is divided into separate IC and EC fluid pathways. In specific aspects, the fluidics for the IC and EC pathways are maintained by inlet pumps that determine the flow of new medium into each side of the bioreactor, and circulation pumps that determine the rate at which the medium in each side of the bioreactor is moved through its circuit. In specific aspects, the cells are seeded onto intracapillary compartments, whereas the EC compartment is used to feed the cells with media.

Generally speaking, appropriate steps are taken to prepare the system prior to loading of the cells, such as preparation of the physical components of the system to facilitate expansion of the cells. The system may be closed or may be semi-closed (as used herein, refers to during the production of exosomes some steps require the opening of the system and the exposure of the sample to the air). Prior to subjecting the cells to be expanded to the system, the bioreactor may be subjected to one or more components and/or one or more conditions to facilitate adherence of cells to the bioreactor. Cell media may be loaded into the system prior to loading of the cells.

For adherent cell production, cells attach and proliferate on the inner surface of each fiber. Suspended cells can be flushed, leaving the adherent cell production for expansion. Automated cell feeding and waste removal means may be part of the system, in specific aspects. In at least some cases, sampling of cells/conditioned media from the system may be provided for without or with interruption of the process. In particular aspects, after cell expansion the adherent cells are released from the hollow fiber walls into suspension, and the suspension including cells and exosomes secreted therefrom are collected.

FIG. 21A demonstrates specific beginning steps that may be employed in a process for generation of desired exosomes. Day 0 in an example of a process may comprise Steps 1, 2, 3, 4, 5, or all 5 of the first 5 Steps. In such a case, Step 1 may comprise a Load Cell Expansion Set Step. In specific aspects, the “Load Cell Expansion Set” step refers to the installation of the disposable cells expansion sets containing the hollow fiber bioreactors (where the cells will grow) onto a Quantum® cell expansion system and the connection of all the lines that allows the supply of CO₂ (for example, 5%), medium, air, and the outline for the waste. During Step 2 of the process is the “Prime Cell Expansion Set” Step, in which the whole hollow fiber system is filled through the inlet and outline connections with phosphate-buffered saline (PBS) without Ca²⁺ and Mg²⁺, removing the air.

During Steps 3 to 5, the bioreactor may be coated prior to loading of the cells, including coating within the hollow fibers of the system. In some cases, in Step 3 a reagent is applied as part of steps for coating a bioreactor. In some cases the bioreactor following application of the reagent is washed (for example, with a buffer such as PBS). In specific cases during Steps 3 to 5, a membrane surface of an intracapillary compartment (e.g., a tubing) of the bioreactor is coated with one or more compounds to promote cell adherence in the bioreactor. In specific cases, the hollow fibers of the bioreactor are coated with human fibronectin (or any extracellular matrix-type reagent, such as RETRONECTIN®) to promote cell adherence. In specific cases, the fibronectin is an extracellular matrix protein (that may be obtained commercially) from human plasma, for example.

In some aspects, Steps 6-8 occur on Day 1. In some cases, Step 6 concerns washing out of the IC and EC pathways (and may entail motion of the sets of the system, including at −90, 180, and 1 degrees of movement of the hollow fiber set)), and Steps 7-8 concern addition of conditioned media to the system (and may have stationary sets of the system). In each of Steps 6-8, IC Media is applied to the EC inlet, but in Step 6 IC Media is also applied to the IC inlet, in some cases. In certain aspects, IC media comprises a source of basic growth media, heparin, platelet lysate, and L-glutamine or a similar compound. In specific aspects, human platelet lysate is utilized because it is a xenogeneic-free, human allogenic replacement for fetal bovine serum, which contains several growth factors useful for cell growth (e.g., epidermal growth factor, platelet derived growth factor, TL-6, insulin-like growth factor, fibroblast growth factor, or a combination thereof) and is obtained from human blood platelets after freeze/thaw cycles. In specific aspects, IC media comprises alpha MEM media supplemented with Heparin 2U/mL, 5% human platelet lysate (hPLT), and 2 mM of GLUTAMAX™

FIG. 21B shows examples of other Steps in Day 1 through Steps in at least part of Day 7 in this example of a process for exosome production. Steps 9-11 concern loading of the cells into the system to produce a uniform suspension in the system. For example, in Step 9, cells may be input into the system through the IC inlet, followed by an appropriate volume of IC media (Step 10); in Step 11, the IC circulation rate may be increased. In each of Steps 9-11, the expansion set may be subjected to motion, such as at −90, 180, and 1. In Step 12, the cells may be allowed to attach upon ceasing motion of the rocker and allowing stationary conditions to support adherence of the cells within the hollow fibers of the sets. Step 12 includes input of IC media to the EC inlet, in particular aspects.

Days 2-5 may include Steps 13-16, respectively, of the process in which the cells are allowed to expand, including in a stationary setting. IC Media is provided through the IC inlet, and in specific cases the IC Inlet Rate is gradually increased over the course of Steps 13-16. In specific aspects, IC Media is not input into the EC Inlet in Steps 13-17. Step 17 on Day 6 includes a wash step, e.g., with a buffer such as PBS.

FIG. 21C shows examples of Steps 20-31 across the course of Days 7-15, in specific aspects. In Step 20, EC media that is platelet-free is input into the EC Inlet, and in the following Step 21 the suspension is harvested following input of IC media into the IC inlet. Harvesting steps may continue periodically thereafter, such as every day, every 2 days, every 3 days, every 4 days, every 5 days, and so on.

Once the cells are harvested from the process, including from the system in such cases, the exosomes may be separated by any suitable means from the supernatant and cells. In some cases, there are multiple harvests from the process, and the supernatant, cells, and exosomes from the process may be pooled prior to any further separation or modification steps. In certain cases, exosomes from multiple harvests are processed separately and combined later.

In some aspects, the exosomes are enriched or concentrated following the production process. As one example, the exosomes are separated from cells, cell fragments, and/or larger or smaller vesicles through physical and/or chemical means. In specific cases, the exosomes are concentrated through one or more centrifugations, one or more filtrations (such as ultrafiltration and/or diafiltration), one or more of immunoisolation, chemical precipitation, size exclusion chromatography, microfluidics, or a combination thereof. Different centrifugation steps may occur at different speeds, and/or different filtration steps may occur at different sizes.

In some aspects, exosomes are enriched or concentrated from the medium of cultured MSCs using differential ultracentrifugation. In some aspects, differential ultracentrifugation comprises the following steps: 1) the supernatant is centrifuged at 2000×g for 20 minutes, and the pellet comprising cells is discarded; 2) the supernatant is filtrated using at 0.2 μm filter; 3) the supernatant is centrifuged at 100000×g for 240 minutes, and the pellet comprising exosomes and cell proteins is obtained and washed in PBS; and 4) the PBS-washed pellet comprising exosomes and cell proteins is centrifuged at 100000×g for 70-180 minutes, and the pellet comprising exosomes is obtained.

Although differential ultracentrifugation provides reasonably pure exosomes, in some aspects, an extra purification step is performed using a sucrose cushion. Thus, in some aspects, exosomes are enriched or concentrated from the medium of cultured MSCs using differential ultracentrifugation followed by filtration through a sucrose gradient. Using a sucrose cushion eliminates more contaminants, such as proteins nonspecifically associated with exosomes, or large protein aggregates, which are sedimented by centrifugation but do not float on a sucrose gradient. Therefore, in some aspects, the recited differential ultracentrifugation steps further comprise the following steps: 5) resuspend partially purified exosome pellet in PBS total; 6) load Tris/sucrose/heavy water (D20) solution at the bottom of a centrifuge tube, to make a cushion; 7) add the diluted exosomes gently above the sucrose cushion without disturbing the interface, and centrifuge 75 minutes at 100,000×g at 4° C.; 8) with a 5-ml syringe fitted with an 18-G needle, collect ˜3.5 ml of the Tris/sucrose/D20 cushion, which now contains exosomes, from the side of the tube; 9) transfer the exosomes to a fresh ultracentrifuge tube, dilute with PBS, and centrifuge 70 min at 100,000×g, at 4° C.; and 10) resuspend the pellet in PBS.

The exosomes may be used immediately or substantially immediately, or they may be stored prior to use, for example at −80° C. or in liquid nitrogen.

In some aspects, the exosomes are concentrated prior to modification of any kind, whereas in other cases the exosomes are modified prior to concentration. The exosomes may be analyzed following the production process, following the concentration step, and/or during the process itself. Such analysis includes identifying one or more markers, identifying size, determining concentration, determining one or more specific activities for the exosomes (such as migration or immunosuppression, and/or anti-T cell activity) or a combination thereof.

### II. Modification of Exosomes

Although in some aspects, the exosomes comprise one or more certain characteristics or activities as a result of being produced from MSCs (including particular MSCs, such as from umbilical cord tissue), the exosomes may be further modified. In particular cases, the exosomes are further modified to harbor (carry) one or more therapeutic agents. In some cases, the MSCs are modified (e.g., transfected, transduced, electroporated, etc.), and modified exosomes are generated by the modified MSCs. In some cases, the exosomes themselves are modified (e.g., transfected, transduced, electroporated, etc.).

The modification of the exosomes may occur by any suitable method in the art, but in specific cases the exosomes are loaded with one or more therapeutic agents by a vector, electroporation, transfection, using a cationic liposome transfection agent, or a combination thereof. Additionally, or alternatively, in some aspects, MSCs are modified by any suitable method in the art, but in specific cases the MSCs are loaded with one or more therapeutic agents by a vector, electroporation, transfection, using a cationic liposome transfection agent, or a combination thereof, and exosomes comprising the one or more therapeutic agents are generated from the modified MSCs.

The therapeutic agent(s) loaded into the exosomes in particular aspects are exogenous with respect to the MSCs. They can be introduced into the exosomes by a number of different techniques. In particular aspects of the disclosure, the exosomes are loaded by electroporation or the use of a transfection reagent.

In specific aspects, the exosomes are of a specific size such that their size determines the type of therapeutic agents that they can carry. In particular cases, the exosomes are 30-400 nm in size, including 30-350, 30-300, 30-250, 30-200, 30-150, 30-100, 30-50, 50-400, 50-350, 50-300, 50-250, 50-200, 50-150, 50-100, 100-400, 100-350, 100-300, 100-250, 100-200, 200-400, 200-350, 200-300, 200-250, 250-400, 250-350, 250-300, 300-400, 300-350, or 350-400 nm in size, or any range or value derivable therein. In some aspects, the exosomes are at least, at most, or about 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 101 nm, 102 nm, 103 nm, 104 nm, 105 nm, 106 nm, 107 nm, 10⁸ nm, 109 nm, 110 nm, 111 nm, 112 nm, 113 nm, 114 nm, 115 nm, 116 nm, 117 nm, 118 nm, 119 nm, 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, 141 nm, 142 nm, 143 nm, 144 nm, 145 nm, 146 nm, 147 nm, 148 nm, 149 nm, 150 nm, 151 nm, 152 nm, 153 nm, 154 nm, 155 nm, 156 nm, 157 nm, 158 nm, 159 nm, 160 nm, 161 nm, 162 nm, 163 nm, 164 nm, 165 nm, 166 nm, 167 nm, 168 nm, 169 nm, 170 nm, 171 nm, 172 nm, 173 nm, 174 nm, 175 nm, 176 nm, 177 nm, 178 nm, 179 nm, 180 nm, 181 nm, 182 nm, 183 nm, 184 nm, 185 nm, 186 nm, 187 nm, 188 nm, 189 nm, 190 nm, 191 nm, 192 nm, 193 nm, 194 nm, 195 nm, 196 nm, 197 nm, 198 nm, 199 nm, 200 nm, 201 nm, 202 nm, 203 nm, 204 nm, 205 nm, 206 nm, 207 nm, 208 nm, 209 nm, 210 nm, 211 nm, 212 nm, 213 nm, 214 nm, 215 nm, 216 nm, 217 nm, 218 nm, 219 nm, 220 nm, 221 nm, 222 nm, 223 nm, 224 nm, 225 nm, 226 nm, 227 nm, 228 nm, 229 nm, 230 nm, 231 nm, 232 nm, 233 nm, 234 nm, 235 nm, 236 nm, 237 nm, 238 nm, 239 nm, 240 nm, 241 nm, 242 nm, 243 nm, 244 nm, 245 nm, 246 nm, 247 nm, 248 nm, 249 nm, 250 nm, 251 nm, 252 nm, 253 nm, 254 nm, 255 nm, 256 nm, 257 nm, 258 nm, 259 nm, 260 nm, 261 nm, 262 nm, 263 nm, 264 nm, 265 nm, 266 nm, 267 nm, 268 nm, 269 nm, 270 nm, 271 nm, 272 nm, 273 nm, 274 nm, 275 nm, 276 nm, 277 nm, 278 nm, 279 nm, 280 nm, 281 nm, 282 nm, 283 nm, 284 nm, 285 nm, 286 nm, 287 nm, 288 nm, 289 nm, 290 nm, 291 nm, 292 nm, 293 nm, 294 nm, 295 nm, 296 nm, 297 nm, 298 nm, 299 nm, 300 nm, 301 nm, 302 nm, 303 nm, 304 nm, 305 nm, 306 nm, 307 nm, 308 nm, 309 nm, 310 nm, 311 nm, 312 nm, 313 nm, 314 nm, 315 nm, 316 nm, 317 nm, 318 nm, 319 nm, 320 nm, 321 nm, 322 nm, 323 nm, 324 nm, 325 nm, 326 nm, 327 nm, 328 nm, 329 nm, 330 nm, 331 nm, 332 nm, 333 nm, 334 nm, 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm, 354 nm, 355 nm, 356 nm, 357 nm, 358 nm, 359 nm, 360 nm, 361 nm, 362 nm, 363 nm, 364 nm, 365 nm, 366 nm, 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, 393 nm, 394 nm, 395 nm, 396 nm, 397 nm, 398 nm, 399 nm, or 400 nm in size.

### A. Exosome Loading

In some cases, exosomes are modified by loading the MSCs or exosomes with one or more therapeutic agents by a vector, electroporation, transfection using a cationic liposome transfection agent, for example, or a combination thereof.

### 1. Vectors

In one aspect, exosomes may be loaded by transforming or transfecting the MSCs with a nucleic acid construct that expresses the therapeutic agent(s), such that the therapeutic agent(s) are present in the exosomes as the exosomes are produced from the cell.

In another aspect, exosomes may also be loaded by directly transforming or transfecting the exosomes with a nucleic acid construct that expresses the therapeutic agent(s).

In some aspects, the nucleic acid construct encoding the therapeutic agent(s) is comprised in a vector. In some cases, the nucleic acid construct encoding the therapeutic agent(s) is linked to a promoter and incorporated into an expression vector, which is taken up and expressed by cells. The vectors can be suitable for replication and, in some cases, integration in eukaryotes. Typical cloning vectors contain transcription and translation terminators, initiation sequences, and promoters useful for regulation of the expression of the desired nucleic acid sequence. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193). In some aspects, a suitable vector is capable of crossing the blood-brain barrier.

In certain aspects the expression vector may be provided to a cell in the form of a viral vector. Viral vector technology is well known in the art and is described, for example, in Sambrook et al. (2001) Molecular Cloning: A Laboratory Manual, Cold Spring Harbor Laboratory, New York), and in other virology and molecular biology manuals.

A number of viral based systems have been developed for gene transfer into mammalian cells. Viruses that are useful as vectors include, but are not limited to, retroviruses, adenoviruses, adeno-associated viruses, herpes viruses, and lentiviruses (including self-inactivating lentivirus vectors). For example, adenoviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Thus, in some aspects, the nucleic acid encoding the polypeptide sequences is introduced into cells using a recombinant vector such as a viral vector including, for example, a lentivirus, a retrovirus, gamma-retroviruses, an adeno-associated virus (AAV), a herpesvirus, or an adenovirus.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available.

When a vector is maintained in a host cell, the vector can either be stably replicated by the cells during mitosis as an autonomous structure, incorporated within the genome of the host cell, or maintained in the host cell's nucleus or cytoplasm.

Eukaryotic expression cassettes included in the vectors particularly contain (in a 5′-to-3′ direction) regulatory elements including a eukaryotic transcriptional promoter operably linked to a protein-coding sequence, splice signals including intervening sequences, a transcriptional termination/polyadenylation sequence, post-transcriptional regulatory elements, and origins of replication.

### 2. Electroporation

In particular aspects of the disclosure, the MSCs and/or exosomes are loaded by electroporation. As used herein, “electroporation” refers to application of an electrical current or electrical field to facilitate entry of an agent of interest into cells, exosomes, or derivatives thereof. One of skill in the art will understand that any method and technique of electroporation is contemplated by the present disclosure. In some aspects, an electroporation system may be controlled to create electric current and send it through a cell- or exosome-containing solution. In some aspects, a static electroporation apparatus is used. In some aspects, a flow electroporation apparatus is used. In specific aspects, static or flow electroporation is used with parameters described herein.

The process of electroporation generally involves the formation of pores in a cell membrane, or in an exosome, by the application of electric field pulses across a liquid cell suspension containing cells or exosomes. The pulse induces a transmembrane potential that causes the reversible breakdown of the cellular membrane. This action results in the permeation or “pore formation” of the cell membrane, which allows introduction of therapeutic agent(s) into the cells or exosomes.

During the electroporation process, cells or exosomes are often suspended in a liquid media and then subjected to an electric field pulse. The medium may be electrolyte, non-electrolyte, or a mixture of electrolytes and non-electrolytes.

The outcome of an electroporation process is largely controlled by the magnitude of the applied electrical field (EF) pulse and the duration of the pulse. Field strength is measured as the voltage delivered across an electrode gap and may be expressed as kV/cm. Field strength is critical to surpassing the electrical potential of the cell membrane to allow the temporary reversible permeation or pore formation to occur in the cell membrane, and the methods of the present disclosure are capable of subjecting the cells to a range of electric field strengths. Field strength is a function of several factors, including voltage magnitude of an applied electrical pulse, duration of the electrical pulse, and conductivity of the sample being electroporated.

The conductivity of the sample is a function of parameters comprising an ionic composition of electroporation buffer, concentration of an agent to be loaded, cell or exosome density, temperature, and pressure. Ionic strength of an electroporation buffer has a direct effect on the resistance of the sample, which in turn affects the pulse length or time constant of the pulse. The size and concentration of an agent will have an effect on the electrical parameters used to transfect the cell. Smaller molecules (for example, siRNA or miRNA) may need higher voltages with microsecond pulse lengths, while larger molecules (for example, DNA and proteins) may need lower voltages with longer pulse lengths. Cell or exosome density can be related to cell size. Generally, smaller cell or exosome sizes require higher voltages while larger cell or exosome sizes require lower voltages for successful cell membrane permeation.

Pulse duration, or pulse length, is the duration of time the sample is exposed to an electrical pulse and is typically measured as time in micro to milliseconds ranges. The pulse length works indirectly with the field strength to increase pore formation and therefore the uptake of target molecules. Generally, an increase in voltage should be followed by an incremental decrease in pulse length. Decreasing the voltage, the reverse is true. In addition to pulse duration, electrical pulses can also be characterized by pulse number, pulse width, pulse shape, pulse pattern, and pulse polarity. Thus, in some aspects, the first and second electrical pulses further comprise characteristics selected from the group consisting of pulse number, width, shape, pattern, and polarity. Electroporation can be carried out as a single pulse or as multiple pulses as disclosed herein to achieve maximum transfection efficiencies. Pulse pattern can comprise a single pulse or multiple pulses, and a combined duration of the multiple pulses corresponds to the pulse duration. Pulse polarity can be positive or negative. Pulse width depends on the wave shape generated by a pulse generator of an electroporation system. Pulse shape, or wave form, generally falls into two categories, square wave or exponential decay wave. Square wave pulses rise quickly to a set voltage level and maintain this level during the duration of the set pulse length before quickly turning off. Exponential decay waves generate an electrical pulse by allowing a capacitor to completely discharge. A pulse is discharged into a sample, and the voltage rises rapidly to the peak voltage set then declines over time. The pulse width in an exponential decay wave system corresponds to the time constant and is characterized by the rate at which the pulsed energy or voltage is decayed to ⅓ the original set voltage. The time constant is modified by adjusting the resistance and capacitance values in an exponential decay, and the calculation for the time is T=RC, where T is time and R is resistance of a sample and C is capacitance of an electroporation system power supply. Thus, in some aspects, the rate of exponential decay is a function of a resistance of the sample and the capacitance of a power supply used to effect electroporation.

The strength of the electric field applied to the suspension and the length of the pulse (the time that the electric field is applied to a cell suspension) varies according to the cell or exosome type. To create a pore in the outer membrane of a cell or exosome, the electric field must be applied for such a length of time and at such a voltage as to increase permeability of the membrane to allow the therapeutic agent(s) to enter the cell or exosome. As long as the pulse magnitude is above a certain threshold level, an increase in either the magnitude or the duration of the pulse generally results in a greater accumulation of the therapeutic agent(s) inside the cell or exosomes.

Each electrical pulse applied to a cell suspension can be characterized by a certain amount of energy, which is equal to the product of voltage on the electrodes, current through the buffer, and duration of the high voltage pulse.

Electroporation parameters may be adjusted to optimize the strength of the applied electrical field and/or duration of exposure such that the pores formed in membranes by the electrical pulse reseal after a short period of time, during which therapeutic agent(s) have a chance to enter into the cell or exosome.

Electroporation conditions may vary depending on the charge and size of the therapeutic agent(s). Typical field strengths are in the range of 20 to 1000 V/cm or kV/cm, such as 20 to 100 V/cm or kV/cm. In some aspects, field strengths are 0.01 to 10, 0.01 to 1, 0.1 to 10, 0.1 to 1, or 1 to 10 V/cm or kV/cm, or any value from 0.01 to 10 V/cm or kV/cm or range derivable therein. In some aspects, field strengths are at least, at most, or about 20 V/cm, 30 V/cm, 40 V/cm, 50 V/cm, 60 V/cm, 70 V/cm, 80 V/cm, 90 V/cm, 100 V/cm, 110 V/cm, 120 V/cm, 130 V/cm, 140 V/cm, 150 V/cm, 160 V/cm, 170 V/cm, 180 V/cm, 190 V/cm, 200 V/cm, 210 V/cm, 220 V/cm, 230 V/cm, 240 V/cm, 250 V/cm, 260 V/cm, 270 V/cm, 280 V/cm, 290 V/cm, 300 V/cm, 310 V/cm, 320 V/cm, 330 V/cm, 340 V/cm, 350 V/cm, 360 V/cm, 370 V/cm, 380 V/cm, 390 V/cm, 400 V/cm, 410 V/cm, 420 V/cm, 430 V/cm, 440 V/cm, 450 V/cm, 460 V/cm, 470 V/cm, 480 V/cm, 490 V/cm, 500 V/cm, 510 V/cm, 520 V/cm, 530 V/cm, 540 V/cm, 550 V/cm, 560 V/cm, 570 V/cm, 580 V/cm, 590 V/cm, 600 V/cm, 610 V/cm, 620 V/cm, 630 V/cm, 640 V/cm, 650 V/cm, 660 V/cm, 670 V/cm, 680 V/cm, 690 V/cm, 700 V/cm, 710 V/cm, 720 V/cm, 730 V/cm, 740 V/cm, 750 V/cm, 760 V/cm, 770 V/cm, 780 V/cm, 790 V/cm, 800 V/cm, 810 V/cm, 820 V/cm, 830 V/cm, 840 V/cm, 850 V/cm, 860 V/cm, 870 V/cm, 880 V/cm, 890 V/cm, 900 V/cm, 910 V/cm, 920 V/cm, 930 V/cm, 940 V/cm, 950 V/cm, 960 V/cm, 970 V/cm, 980 V/cm, 990 V/cm, 1000 V/cm, or any range or value derivable therein.

Field strength is a function of several factors, including voltage magnitude of an applied electrical pulse, duration of the electrical pulse, and conductivity of the sample being electroporated.

A voltage in the range of 150 mV or V to 250 mV or V, particularly a voltage of 200 mV or V may be used for loading exosomes with therapeutic agent(s) according to the present disclosure. In some aspects, the voltage magnitude of the electrical pulses is at most or at least about 0.001 to 10,000, 0.01 to 10,000, 0.1 to 10,000, 1 to 10,000, 1 to 9,000, 1 to 8,000, 1 to 7,000, 1 to 6,000, 1 to 5,000, 1 to 4,000, 1 to 3,000, 1 to 2,000, or 1 to 1,000 mV or V, or any value from 0.001 to 10,000 mV or V or range derivable therein. In some aspects, the voltage magnitude of the electrical pulses is between 0.001 and 10,000, 0.01 and 10,000, 0.1 and 10,000, 1 and 10,000, 1 and 9,000, 1 and 8,000, 1 and 7,000, 1 and 6,000, 1 and 5,000, 1 and 4,000, 1 and 3,000, 1 and 2,000, or 1 and 1,000 mV or V, or any value from 0.001 to 10,000 mV or V or range derivable therein. In some aspects, the voltage magnitude of the electrical pulses can be, can be about, be at least, or be at most 0.001, 0.010, 0.020, 0.030, 0.040, 0.050, 0.060, 0.070, 0.080, 0.090, 0.100, 0.110, 0.120, 0.130, 0.140, 0.150, 0.160, 0.170, 0.180, 0.190, 0.200, 0.210, 0.220, 0.230, 0.240, 0.250, 0.260, 0.270, 0.280, 0.290, 0.300, 0.310, 0.320, 0.330, 0.340, 0.350, 0.360, 0.370, 0.380, 0.390, 0.400, 0.410, 0.420, 0.430, 0.440, 0.450, 0.460, 0.470, 0.480, 0.490, 0.500, 0.510, 0.520, 0.530, 0.540, 0.550, 0.560, 0.570, 0.580, 0.590, 0.600, 0.610, 0.620, 0.630, 0.640, 0.650, 0.660, 0.670, 0.680, 0.690, 0.700, 0.710, 0.720, 0.730, 0.740, 0.750, 0.760, 0.770, 0.780, 0.790, 0.800, 0.810, 0.820, 0.830, 0.840, 0.850, 0.860, 0.870, 0.880, 0.890, 0.900, 0.910, 0.920, 0.930, 0.940, 0.950, 0.960, 0.970, 0.980, 0.990, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 milli-Volts or Volts, or any range or value derivable therein.

In some aspects, the conductivity of the sample is a function of parameters comprising an ionic composition of electroporation buffer, concentration of an agent to be loaded into the cells, cell density, temperature, and pressure. In some aspects, the conductivity of the sample can be, be at least, or be at most 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 Siemens/meter, or any range or value derivable therein. In some aspects, the conductivity of the sample is at most or at least about 0.01 Siemens/meter to 10 Siemens/meter, 0.01 Siemens/meter to 1 Siemens/meter, 0.1 Siemens/meter to 10 Siemens/meter, 0.1 Siemens/meter to 1 Siemens/meter, 1 Siemens/meter to 10 Siemens/meter, or any value from 0.01 Siemens/meter to 10 Siemens/meter or range derivable therein. In some aspects, the conductivity of the sample is between 0.01 Siemens/meter and 10 Siemens/meter, 0.01 Siemens/meter and 1 Siemens/meter, 0.1 Siemens/meter and 10 Siemens/meter, 0.1 Siemens/meter and 1 Siemens/meter, 1 Siemens/meter and 10 Siemens/meter, or any value from 0.01 Siemens/meter to 10 Siemens/meter or range derivable therein. In some aspects, the conductivity of the sample is between 1.0 and 3.0 Siemens/meter, any value from 1.0 Siemens/meter to 3.0 Siemens/meter, or any range or value derivable therein.

The ionic composition of a buffer used for electroporation can vary depending on the cell type. For example, highly conductive buffers such as PBS (Phosphate Buffered Saline <30 ohms) and HBSS (Hepes Buffer <30 ohms) or standard culture media, which may contain serum, may be used. Other buffers include hypoosmolar buffers in which cells absorb water shortly before an electrical pulse, which can result in cell swelling and can lower the optimal permeation voltage while ensuring the membrane is more easily permeable. Cells requiring the use of high resistance buffers (>3000 ohms) may require preparation and washing of the cells to remove excess salt ions to reduce the chance of arcing and sample loss. Ionic strength of an electroporation buffer has a direct effect on the resistance of the sample, which in turn affects the pulse length or time constant of the pulse. The volume of liquid in contact with an electrode also has significant effect on sample resistance for ionic solutions, and the resistance of the sample is inversely proportional to the volume of solution and pH. As volume increases, resistance decreases, which increases the probability of arcing and sample loss, while lowering the volume increases the resistance and decreases arc potential.

The size and concentration of an agent will have an effect on the electrical parameters used to transfect the cell. Smaller molecules (for example, siRNA or miRNA) may need higher voltages with microsecond pulse lengths, while larger molecules (for example, DNA and proteins) may need lower voltages with longer pulse lengths. The concentration of a therapeutic agent may be, may be at least, may be at most, or may be from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 50, 75, 100, 150, 200, 250, 300 to about 350, 400, 500, 1000, 1500, 2000, 3000, 4000, or 5000 μg/mL, mg/mL, or g/mL, or any value from 0.01 to 5000 μg/mL, mg/mL, or g/mL or range derivable therein. In further aspects, the concentration of the therapeutic agent is at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 150, 125, 150, 175, 200, 225, 250, 275, or 300 μg/mL, mg/mL, or g/mL, or any value from 1 to 300 μg/mL, mg/mL, or g/mL or range derivable therein. In certain aspects, the concentration of the therapeutic agent is at least 1 μg/mL, mg/mL, or g/mL. In some aspects, concentration of the therapeutic agent is between 1 μg/mL and 200 μg/mL, such as between 5 μg/mL and 100 μg/mL, any value from 5 μg/mL and 100 μg/mL, or any range derivable therein.

Cell density can be related to cell size. Generally, smaller cell sizes require higher voltages while larger cell sizes require lower voltages for successful cell membrane permeation.

The temperature at which cells are maintained during electroporation can affect the efficiency of the electroporation. Samples pulsed at high voltage or exposed to multiple pulses and long pulse durations can cause sample heating, which can contribute to increased cell death and lower transfection efficiency. Maintaining the sample at a lower temperature can diminish the effects of overheating on cell viability and efficiency. In general, the standard pulse voltage used for cells at room temperature should be approximately doubled for electroporation at 4° C. in order to effectively permeate the cell membrane.

Pulse width depends on the wave shape generated by a pulse generator of an electroporation system. Pulse shape, or wave form, generally falls into two categories, square wave or exponential decay wave. Square wave pulses rise quickly to a set voltage level and maintain this level during the duration of the set pulse length before quickly turning off. In some aspects, the pulse generator generates a square wave pulse, and pulse width can be inputted directly. Exponential decay waves generate an electrical pulse by allowing a capacitor to completely discharge. A pulse is discharged into a sample, and the voltage rises rapidly to the peak voltage set then declines over time. In some aspects, the pulse generator generates an exponential decay wave pulse, and the pulse width is a function of a rate of exponential decay.

The pulse width in an exponential decay wave system corresponds to the time constant and is characterized by the rate at which the pulsed energy or voltage is decayed to ⅓ the original set voltage. The time constant is modified by adjusting the resistance and capacitance values in an exponential decay, and the calculation for the time is T=RC, where T is time and R is resistance of a sample and C is capacitance of an electroporation system power supply. Thus, in some aspects, the rate of exponential decay is a function of a resistance of the sample and the capacitance of a power supply used to effect electroporation.

The resistance of a sample can be, can be at least, or can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 ohms, or any range or value derivable therein. The resistance of a sample can be at most or at least 1 ohm to 10000 ohms, 1 ohm to 9000 ohms, 1 ohm to 8000 ohms, 1 ohm to 7000 ohms, 1 ohm to 6000 ohms, 1 ohm to 5000 ohms, 1 ohm to 4000 ohms, 1 ohm to 3000 ohms, 1 ohm to 2000 ohms, 1 ohm to 1000 ohms, 1 ohm to 900 ohms, 1 ohm to 800 ohms, 1 ohm to 700 ohms, 1 ohm to 600 ohms, 1 ohm to 500 ohms, 1 ohm to 400 ohms, 1 ohm to 300 ohms, 1 ohm to 200 ohms, 1 ohm to 100 ohms, 1 ohm to 90 ohms, 1 ohm to 80 ohms, 1 ohm to 70 ohms, 1 ohm to 60 ohms, 1 ohm to 50 ohms, 1 ohm to 40 ohms, 1 ohm to 30 ohms, 1 ohm to 20 ohms, 1 ohm to 10 ohms, or any value from 1 ohm to 10000 ohms or range derivable therein. In some aspects, the resistance of the sample is between 1 ohm and 10000 ohms, 1 ohm and 9000 ohms, 1 ohm and 8000 ohms, 1 ohm and 7000 ohms, 1 ohm and 6000 ohms, 1 ohm and 5000 ohms, 1 ohm and 4000 ohms, 1 ohm and 3000 ohms, 1 ohm and 2000 ohms, 1 ohm and 1000 ohms, 1 ohm and 900 ohms, 1 ohm and 800 ohms, 1 ohm and 700 ohms, 1 ohm and 600 ohms, 1 ohm and 500 ohms, 1 ohm and 400 ohms, 1 ohm and 300 ohms, 1 ohm and 200 ohms, 1 ohm and 100 ohms, 1 ohm and 90 ohms, 1 ohm and 80 ohms, 1 ohm and 70 ohms, 1 ohm and 60 ohms, 1 ohm and 50 ohms, 1 ohm and 40 ohms, 1 ohm and 30 ohms, 1 ohm and 20 ohms, 1 ohm and 10 ohms, or any value from 1 ohm to 10000 ohms or range derivable therein. In some aspects, the resistance of the sample is between 1 ohm and 1000 ohms, any value from 1 ohm to 1000 ohms, or any range derivable therein.

The power supply capacitance can be at most or at least 1 μF to 1,000 μF, 1 μF to 100 μF, or any value from 1 μF to 1,000 μF or range derivable therein. In some aspects, the power supply capacitance is between 1 μF and 1,000 μF, 1 μF and 100 μF, or any value from 1 μF to 1,000 μF or range derivable therein. In some aspects, the power supply capacitance is between 25 μF and 250 μF, such as between 25 μF and 125 μF, any value from 25 μF and 250 μF, or any range derivable therein. The power supply capacitance can be, can be at least, or can be at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000, 410000, 420000, 430000, 440000, 450000, 460000, 470000, 480000, 490000, 500000, 510000, 520000, 530000, 540000, 550000, 560000, 570000, 580000, 590000, 600000, 610000, 620000, 630000, 640000, 650000, 660000, 670000, 680000, 690000, 700000, 710000, 720000, 730000, 740000, 750000, 760000, 770000, 780000, 790000, 800000, 810000, 820000, 830000, 840000, 850000, 860000, 870000, 880000, 890000, 900000, 910000, 920000, 930000, 940000, 950000, 960000, 970000, 980000, 990000, or 1000000 μF, or any range or value derivable therein.

The therapeutic agents may be proteins and peptides (synthetic, natural, and mimetics, including antibodies or fragments thereof), oligonucleotides (anti-sense oligonucleotides, ribozymes, etc.), short nucleic acid sequences less than about 1000 nucleotides (e.g., double sense linear DNA, inhibitory RNA, siRNA, miRNA, anti-miRNA, shRNA, expression vectors, etc.), ribonucleoproteins, vectors, small molecules, lipids, carbohydrates, cytokines, hemotherapeutic agents, anti-cancer drugs, anti-inflammatory drugs, anti-fungal drugs, anti-viral drugs, anti-microbial drugs, thrombomodulating agents, immunomodulating agents, and the like.

In certain aspects, the therapeutic agent is miRNA, and the concentration of miRNA is between 1 μg/mL and 200 μg/mL, such as between 5 μg/mL and 100 μg/mL, any value from 5 μg/mL and 100 μg/mL, or any range derivable therein.

In certain aspects, the therapeutic agent is siRNA, shRNA, and/or RNA, and the concentration of siRNA, shRNA, and/or RNA is between 1 μg/mL and 200 μg/mL, such as between 10 μg/mL and 50 μg/mL, any value from 10 μg/mL and 50 μg/mL, or any range derivable therein. In certain aspects, the therapeutic agent is siRNA, shRNA, and/or RNA, and the concentration of siRNA, shRNA, and/or RNA is at least, at most, or exactly 1 μg/mL, 2 g/mL, 3 μg/mL, 4 μg/mL, 5 μg/mL, 6 μg/mL, 7 μg/mL, 8 μg/mL, 9 μg/mL, 10 μg/mL, 11 g/mL, 12 μg/mL, 13 μg/mL, 14 μg/mL, 15 μg/mL, 16 μg/mL, 17 μg/mL, 18 μg/mL, 19 μg/mL, g/mL, 21 μg/mL, 22 μg/mL, 23 μg/mL, 24 μg/mL, 25 μg/mL, 26 μg/mL, 27 μg/mL, 28 g/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 g/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 61 μg/mL, 62 g/mL, 63 μg/mL, 64 μg/mL, 65 μg/mL, 66 μg/mL, 67 μg/mL, 68 μg/mL, 69 μg/mL, 70 μg/mL, 71 μg/mL, 72 μg/mL, 73 μg/mL, 74 μg/mL, 75 μg/mL, 76 μg/mL, 77 μg/mL, 78 μg/mL, 79 g/mL, 80 μg/mL, 81 μg/mL, 82 μg/mL, 83 μg/mL, 84 μg/mL, 85 μg/mL, 86 μg/mL, 87 μg/mL, 88 μg/mL, 89 μg/mL, 90 μg/mL, 91 μg/mL, 92 μg/mL, 93 μg/mL, 94 μg/mL, 95 μg/mL, 96 g/mL, 97 μg/mL, 98 μg/mL, 99 μg/mL, 100 μg/mL, 101 μg/mL, 102 μg/mL, 103 μg/mL, 104 g/mL, 105 μg/mL, 106 μg/mL, 107 μg/mL, 10⁸ μg/mL, 109 μg/mL, 110 μg/mL, 111 μg/mL, 112 μg/mL, 113 μg/mL, 114 μg/mL, 115 μg/mL, 116 μg/mL, 117 μg/mL, 118 μg/mL, 119 g/mL, 120 μg/mL, 121 μg/mL, 122 μg/mL, 123 μg/mL, 124 μg/mL, 125 μg/mL, 126 μg/mL, 127 μg/mL, 128 μg/mL, 129 μg/mL, 130 μg/mL, 131 μg/mL, 132 μg/mL, 133 μg/mL, 134 g/mL, 135 μg/mL, 136 μg/mL, 137 μg/mL, 138 μg/mL, 139 μg/mL, 140 μg/mL, 141 μg/mL, 142 μg/mL, 143 μg/mL, 144 μg/mL, 145 μg/mL, 146 μg/mL, 147 μg/mL, 148 μg/mL, 149 g/mL, 150 μg/mL, 151 μg/mL, 152 μg/mL, 153 μg/mL, 154 μg/mL, 155 μg/mL, 156 μg/mL, 157 μg/mL, 158 μg/mL, 159 μg/mL, 160 μg/mL, 161 μg/mL, 162 μg/mL, 163 μg/mL, 164 g/mL, 165 μg/mL, 166 μg/mL, 167 μg/mL, 168 μg/mL, 169 μg/mL, 170 μg/mL, 171 μg/mL, 172 μg/mL, 173 μg/mL, 174 μg/mL, 175 μg/mL, 176 μg/mL, 177 μg/mL, 178 μg/mL, 179 g/mL, 180 μg/mL, 181 μg/mL, 182 μg/mL, 183 μg/mL, 184 μg/mL, 185 μg/mL, 186 μg/mL, 187 μg/mL, 188 μg/mL, 189 μg/mL, 190 μg/mL, 191 μg/mL, 192 μg/mL, 193 μg/mL, 194 g/mL, 195 μg/mL, 196 μg/mL, 197 μg/mL, 198 μg/mL, 199 μg/mL, 200 μg/mL, or any range or value derivable therein.

In certain aspects, the therapeutic agent is DNA, the DNA is at least, at most, or about 1000 base pairs, and the concentration of DNA is between 1 μg/mL and 200 μg/mL, such as between 10 μg/mL and 100 μg/mL, any value from 10 μg/mL and 100 μg/mL, or any range derivable therein. In certain aspects, the therapeutic agent is DNA, the DNA is at least, at most, or about 1000 base pairs, and the concentration of DNA is at least, at most, or exactly 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 11 g/mL, 12 μg/mL, 13 μg/mL, 14 μg/mL, 15 μg/mL, 16 μg/mL, 17 μg/mL, 18 μg/mL, 19 μg/mL, g/mL, 21 μg/mL, 22 μg/mL, 23 μg/mL, 24 μg/mL, 25 μg/mL, 26 μg/mL, 27 μg/mL, 28 g/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 g/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 61 μg/mL, 62 g/mL, 63 μg/mL, 64 μg/mL, 65 μg/mL, 66 μg/mL, 67 μg/mL, 68 μg/mL, 69 μg/mL, 70 μg/mL, 71 μg/mL, 72 μg/mL, 73 μg/mL, 74 μg/mL, 75 μg/mL, 76 μg/mL, 77 μg/mL, 78 μg/mL, 79 g/mL, 80 μg/mL, 81 μg/mL, 82 μg/mL, 83 μg/mL, 84 μg/mL, 85 μg/mL, 86 μg/mL, 87 μg/mL, 88 μg/mL, 89 μg/mL, 90 μg/mL, 91 μg/mL, 92 μg/mL, 93 μg/mL, 94 μg/mL, 95 μg/mL, 96 g/mL, 97 μg/mL, 98 μg/mL, 99 μg/mL, 100 μg/mL, 101 μg/mL, 102 μg/mL, 103 μg/mL, 104 g/mL, 105 μg/mL, 106 μg/mL, 107 μg/mL, 10⁸ μg/mL, 109 μg/mL, 110 μg/mL, 111 Ig/mL, 112 μg/mL, 113 μg/mL, 114 μg/mL, 115 μg/mL, 116 μg/mL, 117 μg/mL, 118 μg/mL, 119 g/mL, 120 μg/mL, 121 μg/mL, 122 μg/mL, 123 μg/mL, 124 μg/mL, 125 μg/mL, 126 μg/mL, 127 μg/mL, 128 μg/mL, 129 μg/mL, 130 μg/mL, 131 μg/mL, 132 μg/mL, 133 μg/mL, 134 g/mL, 135 μg/mL, 136 μg/mL, 137 μg/mL, 138 μg/mL, 139 μg/mL, 140 μg/mL, 141 μg/mL, 142 μg/mL, 143 μg/mL, 144 μg/mL, 145 μg/mL, 146 μg/mL, 147 μg/mL, 148 μg/mL, 149 g/mL, 150 μg/mL, 151 μg/mL, 152 μg/mL, 153 μg/mL, 154 μg/mL, 155 μg/mL, 156 μg/mL, 157 μg/mL, 158 μg/mL, 159 μg/mL, 160 μg/mL, 161 μg/mL, 162 μg/mL, 163 μg/mL, 164 g/mL, 165 μg/mL, 166 μg/mL, 167 μg/mL, 168 μg/mL, 169 μg/mL, 170 μg/mL, 171 μg/mL, 172 μg/mL, 173 μg/mL, 174 μg/mL, 175 μg/mL, 176 μg/mL, 177 μg/mL, 178 μg/mL, 179 g/mL, 180 μg/mL, 181 μg/mL, 182 μg/mL, 183 μg/mL, 184 μg/mL, 185 μg/mL, 186 μg/mL, 187 μg/mL, 188 μg/mL, 189 μg/mL, 190 μg/mL, 191 μg/mL, 192 μg/mL, 193 μg/mL, 194 g/mL, 195 μg/mL, 196 μg/mL, 197 μg/mL, 198 μg/mL, 199 μg/mL, 200 μg/mL, or any range or value derivable therein.

In certain aspects, the therapeutic agent is protein, peptides, lipids, and/or drugs, the protein, peptides, lipids, and the concentration of protein, peptides, lipids, and/or drugs is between 1 μg/mL and 1000 mg/mL, such as between 100 μg/mL and 3 mg/mL, any value from 100 μg/mL and 3 mg/mL, or any range derivable therein. certain aspects, the therapeutic agent is protein, peptides, lipids, and/or drugs, the protein, peptides, lipids, and the concentration of protein, peptides, lipids, and/or drugs is 1 μg/mL or mg/mL, 10 μg/mL or mg/mL, 20 μg/mL or mg/mL, 30 μg/mL or mg/mL, 40 μg/mL or mg/mL, 50 μg/mL or mg/mL, 60 μg/mL or mg/mL, 70 μg/mL or mg/mL, 80 μg/mL or mg/mL, 90 μg/mL or mg/mL, 100 μg/mL or mg/mL, 110 μg/mL or mg/mL, 120 μg/mL or mg/mL, 130 μg/mL or mg/mL, 140 μg/mL or mg/mL, 150 μg/mL or mg/mL, 160 μg/mL or mg/mL, 170 μg/mL or mg/mL, 180 μg/mL or mg/mL, 190 μg/mL or mg/mL, 200 μg/mL or mg/mL, 210 μg/mL or mg/mL, 220 μg/mL or mg/mL, 230 μg/mL or mg/mL, 240 μg/mL or mg/mL, 250 μg/mL or mg/mL, 260 μg/mL or mg/mL, 270 μg/mL or mg/mL, 280 μg/mL or mg/mL, 290 μg/mL or mg/mL, 300 μg/mL or mg/mL, 310 μg/mL or mg/mL, 320 μg/mL or mg/mL, 330 μg/mL or mg/mL, 340 μg/mL or mg/mL, 350 μg/mL or mg/mL, 360 μg/mL or mg/mL, 370 μg/mL or mg/mL, 380 μg/mL or mg/mL, 390 μg/mL or mg/mL, 400 μg/mL or mg/mL, 410 μg/mL or mg/mL, 420 μg/mL or mg/mL, 430 μg/mL or mg/mL, 440 μg/mL or mg/mL, 450 μg/mL or mg/mL, 460 μg/mL or mg/mL, 470 μg/mL or mg/mL, 480 μg/mL or mg/mL, 490 μg/mL or mg/mL, 500 μg/mL or mg/mL, 510 μg/mL or mg/mL, 520 μg/mL or mg/mL, 530 μg/mL or mg/mL, 540 μg/mL or mg/mL, 550 μg/mL or mg/mL, 560 μg/mL or mg/mL, 570 μg/mL or mg/mL, 580 μg/mL or mg/mL, 590 μg/mL or mg/mL, 600 μg/mL or mg/mL, 610 μg/mL or mg/mL, 620 μg/mL or mg/mL, 630 μg/mL or mg/mL, 640 μg/mL or mg/mL, 650 μg/mL or mg/mL, 660 μg/mL or mg/mL, 670 μg/mL or mg/mL, 680 μg/mL or mg/mL, 690 μg/mL or mg/mL, 700 μg/mL or mg/mL, 710 μg/mL or mg/mL, 720 μg/mL or mg/mL, 730 μg/mL or mg/mL, 740 μg/mL or mg/mL, 750 μg/mL or mg/mL, 760 μg/mL or mg/mL, 770 μg/mL or mg/mL, 780 μg/mL or mg/mL, 790 μg/mL or mg/mL, 800 μg/mL or mg/mL, 810 μg/mL or mg/mL, 820 μg/mL or mg/mL, 830 μg/mL or mg/mL, 840 μg/mL or mg/mL, 850 μg/mL or mg/mL, 860 μg/mL or mg/mL, 870 μg/mL or mg/mL, 880 μg/mL or mg/mL, 890 μg/mL or mg/mL, 900 μg/mL or mg/mL, 910 μg/mL or mg/mL, 920 μg/mL or mg/mL, 930 μg/mL or mg/mL, 940 μg/mL or mg/mL, 950 μg/mL or mg/mL, 960 μg/mL or mg/mL, 970 μg/mL or mg/mL, 980 μg/mL or mg/mL, 990 μg/mL or mg/mL, 1000 μg/mL or mg/mL, or any range or value derivable therein.

In particular aspects, the parameters for an electroporation pulse comprise a power between 100 to 240 VAC, with a frequency of between 50 to 60 Hz and a voltage of about 1500 V, with a limitation of 100 A.

In some aspects, all components for electroporation, including but not limited to buffers, exosomes and cuvettes or electrodes, should be kept at at least about 4° C. In some aspects, the electroporation pulse is performed at at least about 25° C., and the electroporated exosomes are placed at at least about 4° C. following electroporation, for example, immediately following electroporation.

Electroporation is capable of achieving loading, or transfection, efficiencies of therapeutic agent(s) into cells or exosomes of greater than 30%, greater than 40%, greater than 50%, greater than 60%, greater than 70%, greater than 80% or greater than 90% (or any range or value derivable therein). In some aspects, a loading efficiency of therapeutic agent(s) is at least 30%, at least 40%, at least 50%, at least 60%, at least 70%, at least 80%, or at least 90%.

Transfection efficiency can be measured either by the percentage of the cells that express the product of the gene or the secretion level of the product expressed by the gene or by directly measuring concentration of the therapeutic agent(s) in the exosomes using, for example, real-time quantitative PCR (RT-qPCR) or similar quantitative analyses.

### 3. Transfection

In particular aspects of the disclosure, the MSCs and/or exosomes are loaded by use of a transfection reagent. Particular transfection reagents for use in accordance with the present disclosure include cationic lipids and/or liposomes.

The use of lipid formulations is contemplated for the introduction of the therapeutic agent(s) into MSCs and/or exosomes. In another aspect, the therapeutic agent(s) may be associated with a lipid. The therapeutic agent(s) associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid.

Lipid-, lipid/DNA-, lipid/expression vector-, or lipid/therapeutic agent(s)-associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a certain aspect, the therapeutic agent(s) may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The amount of liposomes used may vary upon the nature of the liposome as well as the entity to be transfected, for example, about 5 to about 20 pg vector DNA per 1 to 10 million of cells may be contemplated.

Liposome-mediated therapeutic agent(s) delivery and expression of therapeutic agent(s) in vitro has been very successful (Nicolau and Sene, 1982; Fraley et al., 1979; Nicolau et al., 1987). The feasibility of liposome-mediated delivery and expression of therapeutic agent(s) in cultured chick embryo, HeLa and hepatoma cells has also been demonstrated (Wong et al., 1980). In certain aspects, a liposome may be complexed with a hemagglutinating virus (HVJ). This has been shown to facilitate fusion with the cell membrane and promote cell entry of liposome-encapsulated DNA (Kaneda et al., 1989). In other aspects, a liposome may be complexed or employed in conjunction with nuclear non-histone chromosomal proteins (HMG-1) (Kato et al., 1991). In yet further aspects, a liposome may be complexed or employed in conjunction with both HVJ and HMG-1. In other aspects, a delivery vehicle may comprise a ligand and a liposome.

In various aspects lipids suitable for use can be obtained from commercial sources. For example, lipofectamine can be obtained from Thermo Fisher Scientific, Waltham, Mass.; dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem- Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5: 505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### B. Therapeutic Agents

In specific aspects, the exosomes are able to be loaded with any type of therapeutic agent(s). Examples of suitable therapeutic agent(s) include bioactive materials. Bioactive materials particularly suited to incorporation into exosomes include, but are not limited to, therapeutic and prophylactic agents. Examples of bioactive materials include, but are not limited to, proteins and peptides (synthetic, natural, and mimetics, including antibodies or fragments thereof), oligonucleotides (anti-sense oligonucleotides, ribozymes, etc.), short nucleic acid sequences less than about 1000 nucleotides (e.g., double sense linear DNA, inhibitory RNA, siRNA, miRNA, anti-miRNA, shRNA, expression vectors, etc.), ribonucleoproteins, vectors, small molecules, lipids, carbohydrates, cytokines, hemotherapeutic agents, anti-cancer drugs, anti-inflammatory drugs, anti-fungal drugs, anti-viral drugs, anti-microbial drugs, thrombomodulating agents, immunomodulating agents, and the like.

It is to be understood that other therapeutic agent(s) can also be introduced into the exosomes. These agents of interest include, but are not limited to, smooth muscle inhibitors, anti-infective agents (e.g., antibiotics, antifungal agents, antibacterial agents, antiviral agents), chemotherapeutic/antineoplastic agents, and the like. The therapeutic agent(s) may be cancer therapeutic agents, therapeutic agents for auto- or alloimmune disease, therapeutic agents for microbial infection, therapeutic agents for heart disease, therapeutic agents for lung disease, therapeutic agents for liver disease, therapeutic agents for kidney disease, therapeutic agents for neurological disease, or a combination thereof. For cancer therapeutic agents, the agent(s) may be, for example, a drug, small molecule, antibody, inhibitory RNA targeting an oncogene, tumor suppressor protein, or a combination or mixture thereof.

In some aspects, the exosomes comprise one or more short DNA sequences and/or one or more short RNA sequences. The one or more short RNA sequences may comprise inhibitory RNA, including miRNA, anti-miRNA, siRNA, shRNA, Morpholino oligomers, or a combination or mixture thereof. A microRNA (“miRNA” or “miR”) refers to a small single-stranded non-coding RNA molecule that functions in RNA silencing and post-transcriptional regulation of gene expression by base-pairing with complementary sequences within mRNA molecules. Upon base-pairing between the miRNA and complementary mRNA molecule, the mRNA molecule is silenced by either cleavage of the mRNA strand into two pieces, destabilization of the mRNA through shortening of its poly(A) tail, and/or less efficient translation of the mRNA into proteins by ribosomes. Anti-miRNA (also known as “anti-miRNA oligonucleotide” or “AMO”) refers to synthetically designed molecules used to neutralize miRNA function in cells. By controlling the miRNA that regulate mRNAs in cells, AMOs can be used as further regulation through, for example, a steric blocking mechanism as well as hybridization to miRNA. These interactions between miRNA and AMOs can be therapeutic in disorders in which miRNA over/under expression occurs or aberrations in miRNA lead to coding issues. Small interfering RNA (“siRNA” or ““short interfering RNA” or “silencing RNA” refers to a class of double-stranded RNA non-coding RNA molecules that operate in sequence-specific suppression of gene expression. siRNA interfere with the expression of specific genes with complementary nucleotide sequences by degrading mRNA after transcription, thereby preventing translation of the mRNA into amino acids and then proteins. siRNA may be introduced into cells using an expression vector in which the siRNA sequence is modified to introduce a short loop between the two strands. The resulting transcript is a short hairpin RNA (“shRNA” or “short hairpin RNA”), which can be processed into a functional siRNA by Dicer, an enzyme that cleaves double-stranded RNA into siRNA (and pre-microRNA into microRNA). Morpholino oligomers (“morpholino” or “phosphorodiamidate morpholino oligomer” or “PMO”) refer to oligomer molecules containing DNA bases attached to a backbone of methylenemorpholine rings linked through phosphorodiamidate groups. Morpholinos sterically block access of other molecules to small specific sequences of the base-pairing surfaces of RNA, thereby modifying gene expression. For example, Morpholinos can modify pre-mRNA splicing, block translation by interfering with progression of the ribosomal initiation complex from the 5′ cap to the start codon, or block other functional sites on RNA (i.e., blocking miRNA activity and maturation, blocking ribozyme activity, etc.) depending on the Morpholino's base sequence.

In some aspects, the exosomes comprise one or more antibodies or antibody fragments. The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). An antibody of use in the invention may be a monoclonal antibody or a polyclonal antibody, and will preferably be a monoclonal antibody. An antibody of use in the invention may be a chimeric antibody, a CDR-grafted antibody, a nanobody, a human or humanized antibody or an antigen binding portion of any thereof. For the production of both monoclonal and polyclonal antibodies, the experimental animal is typically a non-human mammal such as a goat, rabbit, rat or mouse but may also be raised in other species such as camelids.

The term “antigen-binding portion” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a F(ab′)2 fragment, a Fab′ fragment, a Fd fragment, a Fv fragment, a dAb fragment, and an isolated complementarity determining region (CDR). Single chain antibodies such as scFv antibodies are also intended to be encompassed within the term “antigen-binding portion” of an antibody. These antibody fragments may be obtained using conventional techniques known to those of skill in the art, and the fragments may be screened for utility in the same manner as intact antibodies. An antibody of use in the invention may be a human antibody or a humanized antibody.

In some aspects, the exosomes are loaded with one or more cancer drugs, including one or more chemotherapies. A wide variety of chemotherapeutic agents may be used in accordance with the present aspects. The term “chemotherapy” refers to the use of drugs to treat cancer. A “chemotherapy” or “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapies include alkylating agents, such as thiotepa, procarbazine, and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as plicomycin and the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), adriamycin, epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, gemcitabine, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., taxol, paclitaxel, and docetaxel; 6-thioguanine; mercaptopurine; platinum coordination complexes, such as transplatinum, cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; protease inhibitors, like bortezomib; kinase inhibitors, like palbociclib, ibrutinib, dasatinib, pp2, pazopanib, and gefitinib; checkpoint inhibitors, like nivolumab, pembrolizumab, and ipilimumab; colony stimulating factors, like pegfilgrastim and filgrastim; monoclonal antibodies, like bevacizumab, trastuzumab, and rituximab; immunomodulatory agents, like lenalidomide; navelbine; farnesyl-protein transferase inhibitors; pharmaceutically acceptable salts, acids, or derivatives of any of the above; and combinations thereof.

In certain aspects, the exosomes are loaded with one or more antimicrobial agents. An antimicrobial agent may be a natural or synthetic substance that kills or inhibits the growth of microorganisms or pathogens, such as bacteria, fungi, algae, or viruses. The antimicrobial agents may be an antibiotic, antifungal, antiviral, and so forth.

Examples of antibiotics include but are not limited to aminoglycosides, ansamycins, carbacephem, carbapenems, cephalosporins (first, second, third, fourth, or fifth generation), glycopeptides, linocsamides, lipopeptides, macrolides, monobactams, nitrofurans, oxazolidinones, penicillins, polypeptides, quinolones/fluoroquinolones, sulfonamides, tetracyclines, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, trimethoprim, and combinations thereof. Aminoglycosides can include, but are not limited to: Amikacin, Gentamicin, Kanamycin, Neomycin, Netilmicin, Tobramycin, Paromomycin, Streptomycin, and Spectinomycin. Ansamycins can include but are not limited to: Geldanamycin, Herbimycin, and Rifaximin. Carbacephem can include but is not limited to Loracarbef. Carbapenems can include but are not limited to Ertapenem, Doripenem, Imipenem/Cilastatimn, and Meropenem. Cephalosporins can include but are not limited to: Cefadroxil, Cefazolin, Cephradine, Cephapirin, Cephalothin, Cefalexin, Cefaclor, Cefoxitin, Cefotetan, Cefotan, Cefamandole, Cefmetazole, Cefonicid, Loracarbef, Cefprozil, Cefzil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole. Glycopeptides can include but are not limited to: Teicoplanin, Vancomycin, Telavancin, Dalbavancin, and Oritavancin. Lincosamides can include but are not limited to Clindamycin and Lincomycin. Lipopeptides can include but are not limited to Daptomycin. Macrolides can include but are not limited to: Azithromycin, Clarithromycin, Erythromycin, Roxithromycin, Telithromycin, Spiramycin, and Fidaxomicin. Monobactams can include but are not limited to Aztreonam. Nitrofurans can include but are not limited to: Furazolidone and Nitrofurantoin. Oxazolidinones can include but are not limited to: Linezolid, Posizolid, Radezolid, and Torezolid. Penicillins can include but are not limited to: Amoxicillin, Ampicillin, Azlocillin, Dicloxacillin, Flucloxxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, and Ticarcillin/clavulanate. Polypeptides can include but are not limited to: Bacitracin, Colistin, and Polymyxin B. Quinolones/fluoroquinolones can include but are not limited to: Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin. Sulfonamides can include but are not limited to: Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, and Sulfoamidochrysoidine. Tetracyclines can include but are not limited to: Demeclocycline, Doxycycline, Metacycline, Minocycline, Oxytetracycline, and Tetracycline. In some aspects, the antibiotic is a macrolide. In some aspect, the antibiotic is azithromycin.

Examples of antibiotics also include but are not limited to antimicrobial proteins or peptides. The antimicrobial proteins or peptides can be of any class, including but not limited to the following classes: anionic peptides (e.g., dermicidin), linear cationic a-helical peptides (e.g., LL37), cationic peptides enriched for proline, arginine, phenylalanine, glycine, or tryptophan, anionic and cationic peptides that contain cysteine and form disulfide bonds (e.g., defensins), and combinations thereof. Defensins can include but are not limited to trans-defensins, cis-defensins, and related defensin-like proteins. Trans-defensins include but are not limited to a-defensins and P-defensins.

Examples of antibiotics also include but are not limited to anti-mycobacterials, including, but not limited to, isoniazid, rifampin, streptomycin, rifabutin, ethambutol, pyrazinamide, ethionamide, aminosalicylic, and cycloserine.

Examples of antivirals include but are not limited to anti-herpes agents such as acyclovir, famciclovir, foscamet, ganciclovir, acyclovir, idoxuridine, sorivudine, trifluridine, valacyclovir and vidarabine; anti-retroviral agents such as ritonavir, didanosine, stavudine, zalcitabine, tenovovir and zidovudine; and other antiviral agents such as, but not limited to, amantadine, interferon-alpha, ribavirin, rimantadine, and combinations thereof.

Examples of antifungals include but are not limited to polyene antifungals (e.g., amphotericin B, nystatin, natamycin, and the like), flucytosine, imidazoles (e.g., n-ticonazole, clotrimazole, econazole, ketoconazole, and the like), triazoles (e.g., itraconazole, fluconazole, and the like), griseofulvin, terconazole, butoconazole ciclopirax, ciclopirox olamine, haloprogin, tolnaftate, naftifine, terbinafine, any other antifungal that can be lipid encapsulated or complexed, and combinations thereof.

In some aspects, the exosomes are loaded with one or more therapeutic agents for the treatment of an auto- or alloimmune disease. Examples of auto- or alloimmune disease therapies include but are not limited to anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins, or their inhibitors (e.g., BAFF, IL-2, anti-IL-2R, IL-4, JAK kinase inhibitors) can be administered.

In alternative aspects, the exosomes are not loaded with a therapeutic drug but instead are loaded with one or more gene-modifying components, such as that comprise a CRISPR-Cas system, including a specific guide RNA and an endonuclease. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains).

In some aspects, a Cas nuclease and gRNA (including a fusion of crRNA specific for the target sequence and fixed tracrRNA) are introduced into the cell. In general, a guide sequence is any polynucleotide sequence having sufficient complementarity with a target polynucleotide sequence to hybridize with the target sequence and direct sequence-specific binding of the CRISPR complex to the target sequence. Typically, “target sequence” generally refers to a sequence to which a guide sequence is designed to have complementarity, where hybridization between the target sequence and a guide sequence promotes the formation of a CRISPR complex. The CRISPR system can induce double stranded breaks (DSBs) at the target site, followed by disruptions or alterations as discussed herein. In other aspects, Cas9 variants, deemed “nickases,” are used to nick a single strand at the target site. In other aspects, catalytically inactive Cas9 is fused to a heterologous effector domain such as a transcriptional repressor or activator, to affect gene expression. The target sequence may comprise any polynucleotide, such as DNA or RNA polynucleotides. The target sequence may be located in the nucleus or cytoplasm of the cell, such as within an organelle of the cell.

In some aspects, exosomes loaded with one or more vectors can be introduced into cells to drive expression of one or more elements of the CRISPR system such that expression of the elements of the CRISPR system direct formation of the CRISPR complex at one or more target sites. Components can also be delivered to cells via exosomes as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some aspects, one or more insertion sites are located upstream and/or downstream of one or more sequence elements of one or more vectors. When multiple different guide sequences are used, a single expression construct may be used to target CRISPR activity to multiple different, corresponding target sequences within a cell. A vector may comprise a regulatory element operably linked to an enzyme-coding sequence encoding the CRISPR enzyme, such as a Cas protein.

### III. Methods of Using Exosomes

In particular aspects, exosomes are useful for the treatment of one or more medical conditions. The exosomes may be used for the systemic or local delivery of therapeutic compounds. The disclosure encompasses methods for delivering therapeutic agents of interest using exosomes as a delivery vehicle. The present disclosure also includes methods of treating a patient in need of one or more therapeutic agents comprising administering to the patient an effective amount of exosomes containing the therapeutic agent(s).

The exosomes of the disclosure may or may not be utilized directly after production. In some cases they are stored for later purpose. In any event, they may be utilized in therapeutic or preventative applications for a mammalian subject (human, dog, cat, horse, etc.) such as a patient. The individual may be in need of exosome-based therapy for a medical condition of any kind, including cancer, infections of any kind, any immune disorder, any tissue injury, any skin disorder, any wounds, any trauma, and/or any burns, as examples. Methods may be employed with respect to individuals who have tested positive for a medical condition, who have one or more symptoms of a medical condition, or who are deemed to be at risk for developing such a condition.

Individuals treated with the present exosome-based therapy may or may not have been treated for the particular medical condition prior to receiving the exosome-based therapy. In some aspects, the patient has received at least 1, 2, 3, 4, 5, 6, 7, 8, or more prior treatments for a medical condition. The prior treatments may include a treatment or therapy described herein. In some aspects, the prior treatments comprise conventional chemotherapies, conventional radiotherapy, conventional antiviral therapies, conventional antiseptic and antibacterial therapies, conventional immunosuppressive therapies, conventional anti-inflammatory therapies, conventional burn treatment therapies, and the like. In some aspects, the patient had received the prior therapy within 10, 20, 30, 40, 50, 60, 70, 80, or 90 days or hours of administration of the current compositions and exosomes of the disclosure. In some aspects, the patient is one that has undergone prior therapy and has failed the prior treatment either because the prior treatment was not effective or because the prior treatment was deemed too toxic.

Exosomes loaded with one or more therapeutic agents as contemplated herein, and/or pharmaceutical compositions comprising the same, can be administered either alone or in any combination, and in at least some aspects, together with a pharmaceutically acceptable carrier or excipient, and can be used for the prevention, treatment, or amelioration of cancer, immune disorders, heart disease, lung disease, microbial infections, tissue injuries, skin disorders, wounds, trauma, and/or burns of any kind. Exosomes loaded with one or more therapeutic agents as contemplated herein, and/or pharmaceutical compositions comprising the same, can also be used for the mitigation of chemo- and radiotherapy-induced CNS toxicity, and the treatment of other chemotherapy or radiation-induced vital organ toxicities involving the heart, lung, kidney, gastrointestinal tract where regenerative or reparative properties are often needed.

Exosomes loaded with one or more therapeutic agents as contemplated herein, and/or pharmaceutical compositions comprising the same, can also be used for the mitigation of chemo- and radiotherapy-induced CNS toxicity, and the treatment of other chemotherapy or radiation-induced vital organ toxicities involving the heart, lung, kidney, gastrointestinal tract where regenerative or reparative properties are often needed.

Aspects of the disclosure include methods for treating, reversing, or ameliorating cognitive dysfunction in response to or providing neuroprotection against chemo- and radiotherapy-induced central nervous system (CNS) toxicity. In specific aspects, exosomes derived from umbilical cord tissue-derived MSCs (UC-Exos) are useful for treating or reversing cognitive dysfunction in response to or providing neuroprotection against chemo- and radiotherapy-induced central nervous system (CNS) toxicity. Methods and compositions of the disclosure allow for generation of a large scale of activated exosomes from UC-Exos, carrying therapeutic agent(s), including at least miR, anti-miR, siRNA, and therapeutic drugs for treating or reversing cognitive dysfunction in response to or providing neuroprotection against chemo- and radiotherapy-induced central nervous system (CNS) toxicity.

In certain aspects, the exosomes (e.g., UC-Exos) are utilized for individuals in need of regeneration and/or reparation of tissue for any reason. The tissue in need of regeneration and/or reparation may be of any kind, but in specific aspects the tissue is soft tissue (e.g., fat, fibrous tissue (e.g., tendons and/or ligaments), muscle (e.g., smooth muscle, skeletal muscle, and/or cardiac muscle), synovial tissue, blood vessels, lymph vessels, and/or nerves), brain, lung, spleen, liver, heart, kidney, pancreas, intestine, testis, or bone. For example, the individual may be in need of regeneration and/or reparation of heart, lung, kidney, and/or gastrointestinal tract tissue due to chemotherapy or radiation-induced vital organ toxicities. The individual may be in need of regeneration and/or reparation of soft tissue (e.g., fat, fibrous tissue (e.g., tendons and/or ligaments), muscle (e.g., smooth muscle, skeletal muscle, and/or cardiac muscle), synovial tissue, blood vessels, lymph vessels, and/or nerves) due to inflammation, trauma (e.g., contusions, sprains, tendonitis, bursitis, stress injuries, strains), burns (e.g., thermal burns, chemical burns, electric burns, frostbite) or a combination thereof. In some aspects, the tissue is in need of regeneration or repair due to toxicity due to burns (e.g., thermal burns, chemical burns, electric burns, frostbite) or trauma (e.g., contusions, sprains, tendonitis, bursitis, stress injuries, strains) and/or due to toxicity due to a prior treatment for burns (e.g., thermal burns, chemical burns, electric burns, frostbite) or trauma (e.g., contusions, sprains, tendonitis, bursitis, stress injuries, strains). In particular aspects, the exosomes produced by methods encompassed herein are useful as regenerative and/or reparative therapies to target soft tissues and organs including brain, lung, spleen, liver, heart, kidney, pancreas, intestine, testis, and bone, as examples of target tissues. The exosomes in such cases are therapeutic at least in part because they are suitable to migrate in the individual.

In certain aspects, the exosomes (e.g., UC-Exos) are utilized for individuals in need of regeneration and/or reparation of skin for any reason. For example, the individual may be in need of regeneration and/or reparation of skin due to chemotherapy or radiation-induced vital organ toxicities. The individual may be in in need of regeneration and/or reparation of skin due to toxicity due to burns (e.g., thermal burns, chemical burns, electric burns, frostbite) or trauma (e.g., cutaneous wound damage, contusions, cuts, lacerations, gashes, tears, punctures, scrapes, abrasions, scratches, bites, stings, bruises, pressure injuries, crush injuries, incisions) and/or due to toxicity due to a prior treatment administered for burns (e.g., thermal burns, chemical burns, electric burns, frostbite) or trauma (e.g., cutaneous wound damage, contusions, cuts, lacerations, gashes, tears, punctures, scrapes, abrasions, scratches, bites, stings, bruises, pressure injuries, crush injuries, incisions). The individual may be in need of regeneration and/or reparation of skin due to a skin disorder. Non-limiting examples of skin disorders include inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

In certain aspects, the exosomes (e.g., UC-Exos) are utilized for individuals in need of wound healing (e.g., wound repair) for any reason. For example, the individual may be in need of regeneration and/or reparation of wounded skin or tissue due to chemotherapy or radiation-induced vital organ toxicities. The individual may be in need of regeneration and/or reparation of wounded skin or tissue due to toxicity due to burns (e.g., thermal burns, chemical burns, electric burns, frostbite) or trauma (e.g., sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions) and/or toxicity due to a prior treatment for burns (e.g., thermal burns, chemical burns, electric burns, frostbite) or trauma (e.g., sprains, tendonitis, bursitis, stress injuries, strains, cutaneous wound damage, contusions, cuts, lacerations, gashes, tears, punctures, scrapes, abrasions, scratches, bites, stings, bruises, pressure injuries, crush injuries, incisions). The individual may be in need of regeneration and/or reparation of wounded skin or tissue due to inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

Aspects of the disclosure include methods for treatment of cancer. In some cases, the exosomes are useful for one or more cancers. In specific aspects, exosomes derived from umbilical cord tissue-derived MSCs (UC-Exos) are useful for the treatment of cancer and for the systemic delivery of therapeutic compounds for the cancer. Methods and compositions of the disclosure allow for generation of a large scale of activated exosomes from UC-Exos, carrying therapeutic agent(s), including at least miR, anti-miR, siRNA, and therapeutic drugs for the treatment of cancer.

Cancers for which the present exosomes are useful include any malignant cell type, such as those found in a solid tumor or a hematological tumor. In cases wherein the individual has cancer, the cancer may be primary, metastatic, resistant to therapy, and so forth. In specific cases, the present therapy is useful for individuals with cancers that have been clinically indicated to be subject to immune cell regulation, including multiple types of solid tumors (melanoma, colon, lung, breast, and head and neck cancers), for example. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, glioblastoma, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In some aspects, cancers for which the present exosomes are useful is glioblastoma multiforme (GBM). Adult glioblastoma is notoriously recalcitrant to most therapies, not only because its molecular, cellular and immune biology are unique compared with other cancers, but also because of the formidable delivery challenges imposed by the blood brain/blood tumor barriers (BBB/BTB). Consequently, there is an urgent need to identify anti-cancer therapeutics that specifically target GBMs, and to elucidate strategies for delivering these new agents across the BBB/BTB. I some cases, exosomes home efficiently to human gliomas, overcoming the BBB/BTB.

In some aspects, exosomes used to treat GBM are loaded with the anti-GMB miRNA miR-124. Validation studies proved that miR-124 is highly efficacious against all subtypes of glioma stem cells, functioning by down-regulating GBM-relevant targets, particularly FOXA2, and leading to apoptotic cell death. MiR-124 also enhances T-cell responses by inhibiting STAT-3, a known mediator of immune suppression in GBM, further supporting its therapeutic potential. Recent work has also shown that miR-124 reverses neurodegeneration after brain injury, rendering miR-124 one of the first anti-glioma agents that may also mitigate neuro-toxicity.

Aspects of the disclosure include methods for treatment of immune disorders. In some cases, the exosomes are useful for one or more immune disorders. In specific aspects, exosomes derived from umbilical cord tissue-derived MSCs (UC-Exos) are useful for the treatment of immune disorders and for the systemic delivery of therapeutic compounds for the immune disorders. Methods and compositions of the disclosure allow for generation of a large scale of activated exosomes from UC-Exos, carrying therapeutic agent(s), including at least miR, anti-miR, siRNA, and therapeutic drugs, for the treatment of immune disorders.

Immune disorders for which the present exosomes are useful include autoimmune or inflammatory disorders. Non-limiting examples of autoimmune or inflammatory disorders include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, graft versus host disease (GVHD), and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as asthma.

Aspects of the disclosure include methods for treatment of heart disease of any kind, including at least coronary artery disease, heart failure, cardiomyopathy, valvular heart disease, arrhythmia, genetic defects of the heart, and so forth.

Aspects of the disclosure include methods for treatment of lung disease, such as pulmonary hypertension, asthma, bronchopulmonary dysplasia (BPD), allergy, cystic fibrosis, Chronic Obstructive Pulmonary Disease, idiopathic pulmonary fibrosis, acute respiratory distress syndrome (ARDS), pneumonia, pleural effusion, and so forth.

Aspects of the disclosure include methods for treatment of a microbial infection of any kind, including a pathogenic infection. The infection may be bacterial, viral, fungal, or protozoan. Examples of bacterial include, but are not limited to, Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia. Examples of fungi include, but are not limited to, Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. Examples of protozoa include, but are not limited to, Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Examples of helminth parasites include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca, Spirometra, Stephanofilaria, Strongyloides, Strongylus, Thelazia, Toxascaris, Toxocara, Trichinella, Trichostrongylus, Trichuris, Uncinaria, Wuchereria, Pneumocystis, Sarcocystis, Schistosoma, Theileria, Toxoplasma, and Trypanosoma. Examples of viruses include adenovirus, alphavirus, calicivirus, coronavirus (including SARS-CoV, SARS-CoV-2, and MERS), distemper virus, Ebola virus, enterovirus, flavivirus, hepatitis virus, herpesvirus, infectious peritonitis virus, leukemia virus, Marburg virus, Norwalk virus, orthomyxovirus, papilloma virus, parainfluenza virus., the, paramyxovirus, parvovirus, pestivirus, picorna virus, pox virus, rabies virus, reovirus polypeptides, retrovirus, rotavirus, and vaccinia virus.

The exosome compositions of the disclosure may be administered by any suitable means. Administration to a human or animal subject may be selected from rectal, buccal, vaginal, parenteral, intramuscular, intracerebral, intravascular (including intravenous), intracutaneous, subcutaneous, intranasal, intracardiac, intracerebroventricular, intraperitoneal intraarticular, intrasynovial, intrasternal, intrathecal, intralesional, or intracranial routes, or transdermal administration, or via an implanted reservoir.

The exosomes may be delivered as a composition. The composition may be formulated for any suitable means of administration, including rectal, buccal, vaginal, parenteral, intramuscular, intracerebral, intravascular (including intravenous), intracutaneous, subcutaneous, intranasal, intracardiac, intracerebroventricular, intraperitoneal intraarticular, intrasynovial, intrasternal, intrathecal, intralesional, or intracranial routes, or transdermal administration, or via an implanted reservoir. Compositions for parenteral administration may include sterile aqueous solutions which may also contain buffers, diluents and other suitable additives. The exosomes of the disclosure may be formulated in a pharmaceutical composition, which may include pharmaceutically acceptable carriers, thickeners, diluents, buffers, preservatives, and other pharmaceutically acceptable carriers or excipients and the like in addition to the exosomes.

A “pharmaceutically acceptable carrier” (excipient) is a pharmaceutically acceptable solvent, suspending agent or any other pharmacologically inert vehicle for delivering one or more nucleic acids to a subject. Typical pharmaceutically acceptable carriers include, but are not limited to, binding agents (e.g. pregelatinised maize starch, polyvinylpyrrolidone or hydroxypropyl methylcellulose, etc.); fillers (e.g. lactose and other sugars, microcrystalline cellulose, pectin, gelatin, calcium sulfate, ethyl cellulose, polyacrylates or calcium hydrogen phosphate, etc.); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc.); or wetting agents (e.g. sodium lauryl sulphate, etc.).

The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions provided herein.

A therapeutically effective amount of composition is administered. The therapeutically effective amount of the produced exosomes is that amount that achieves a desired effect in a subject being treated. For instance, this can be the amount of exosomes necessary to inhibit advancement, or to cause regression of, cancer, or which is capable of relieving symptoms caused by cancer. This can be the amount of exosomes necessary to inhibit advancement, or to cause regression, of an autoimmune or alloimmune disease, or which is capable of relieving symptoms caused by an autoimmune disease, such as pain and inflammation. It can also be of the amount of exosomes necessary to inhibit advancement, or to cause regression, of a microbial infection, or which is capable of relieving symptoms caused by a microbial infection.

The produced exosomes can be administered in treatment regimens consistent with the disease, for example a single or a few doses over one to several days to ameliorate a disease state or periodic doses over an extended time to inhibit disease progression and prevent disease recurrence. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC₅₀s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection. In some cases, the dose of single or multiple systemic injections is in the range of 10 to 100 mg/kg of body weight.

In some cases, the individual may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the individual undergo maintenance therapy, wherein the construct is administered in maintenance doses, ranging from 0.01 mg/kg to 100 mg per kg of body weight, once or more daily, to once every 20 years.

### IV. Kits

Any of the compositions described herein may be comprised in a kit. In a non-limiting example, cells, reagents to produce cells, exosomes, and reagents to produce exosomes, and/or components thereof may be comprised in a kit. In certain aspects, exosomes may be comprised in a kit, and they may or may not yet express one or more therapeutic agents. Such a kit may or may not have one or more therapeutic agents to be loaded into the exosomes, including reagents to generate same and/or reagents to manipulate the exosomes for loading of the agents. Such agents include small molecules, proteins, nucleic acids, antibodies, buffers, primers, nucleotides, salts, and/or a combination thereof, for example.

In particular aspects, the kit comprises the exosome-based therapy of the disclosure and also another therapy. In some cases, the kit, in addition to the exosome-based therapy aspects, also includes a second therapy, such as chemotherapy, hormone therapy, immunotherapy, and/or antimicrobial therapy, for example. The kit(s) may be tailored to a particular disease for an individual and comprise respective second therapies for the individual.

The article of manufacture or kit can further comprise a package insert comprising instructions for using the exosomes to treat or delay progression of disease, for example, cancer, an infection, or an immune disorder, in an individual or to enhance treatment of an individual having cancer, an infection, or an immune disorder. Any of the exosomes described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or HASTELLOY®). In some aspects, the container holds the formulation and the label on, or associated with, the container may indicate directions for use.

The article of manufacture or kit may further include other materials desirable from a commercial and user standpoint, including other buffers, diluents, filters, needles, syringes, and package inserts with instructions for use. In some aspects, the article of manufacture further includes one or more of another agent (e.g., a chemotherapeutic agent, an anti-neoplastic agent, an anti-microbial agent). Suitable containers for the one or more agent include, for example, bottles, vials, bags and syringes.

### EXAMPLES

The following examples are included to demonstrate aspects of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific aspects which are disclosed and still obtain a like or similar result without departing from the spirit and scope of the disclosure.

### Example 1

### A Platform-Approach Using Exosomes as a Delivery Vehicle for Therapeutic Cargoes

The present example provides a novel, robust, GMP-compliant platform to generate exosomes from umbilical cord-derived mesenchymal stem/stromal cells (UC-MSCs) or bone marrow-derived mesenchymal stem/stromal cells (BM-MSCs) and to load them with highly potent and novel therapeutics, including miR-124, other therapeutic miRs (e.g., miR-135-a-2, Let7i, miR-148, -375, -657, -668, -485, -520d, -569, -943, -1181, -198, -521-1, -24-1), anti-tumoral miRs for other cancers, and siRNAs (e.g., siRNAs against gene fusions such as FGFR-TACC fusions).

In particular, the inventors developed an approach using lentivral transduction of the miR-124 was lentivirally transduced into MSCs from which the exosomes were generated as well as an efficient and GMP-compliant approach to efficiently load therapeutics into MSC-Exos and have shown that loaded MSC-Exos are highly effective against a wide range of molecularly heterogeneous GSCs in vitro. In pivotal preclinical in vivo mouse studies, the inventors demonstrated that loaded MSC-Exos can eradicate intracranial human gliomas after systemic delivery and down-regulate protein and nucleic acid targets. The novelty and potential clinical impact is, in some aspects, the duality of this therapeutic to eradicate the GBM while simultaneously ameliorating chemo- and radiation therapy-induced brain damage. This approach can serve as a platform for many different therapeutics, including miRs, siRNAs, or combinations thereof, as well as other therapeutic genes. The established method is practical, efficient, and in some aspects, allows for the clinical use of BM or UC-MSC-exosomes as therapeutic agents for the treatment of patients with GBM and other solid and liquid tumors, as well as for patients with alloimmune or autoimmune disorders, tissue injuries, skin disorders, wounds, as vehicles for gene and drug delivery, and as therapeutic agents in regenerative medicine settings.

1. Identification of miRs for Treating Glioblastoma

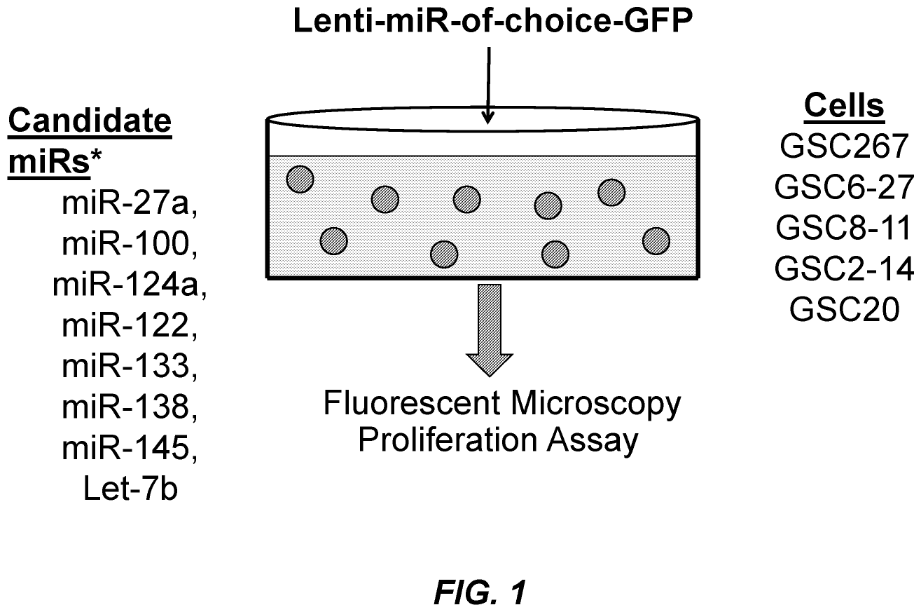

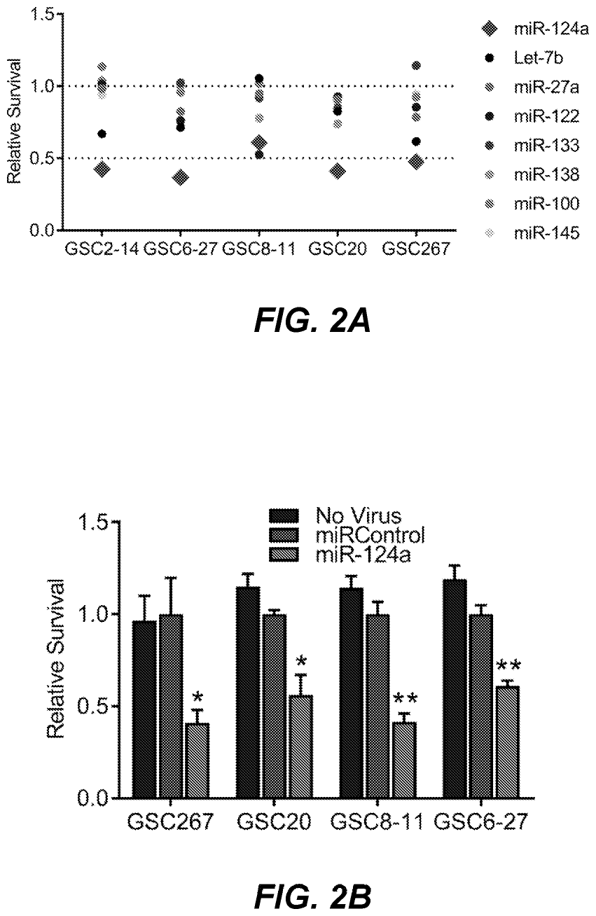

To identify effective miRs for treating GBMs, eight miRs (miR-27a, miR-100, miR-124, miR-122, miR-133, miR-138, miR-145, Let-7b) were selected and screened for their anti-glioma effects against five glioma stem cell lines (GSCs), representing all TCGA-defined GBM subtypes (GSC267, GSC6-27, GSC8-11, GSC2-14, GSC20) (FIG. 1). MiR-124 resulted in the greatest decrease in viability in all GSCs (P<0.010), identifying miR-124a as a highly effective anti-GBM miR (FIGS. 2A, 2B).

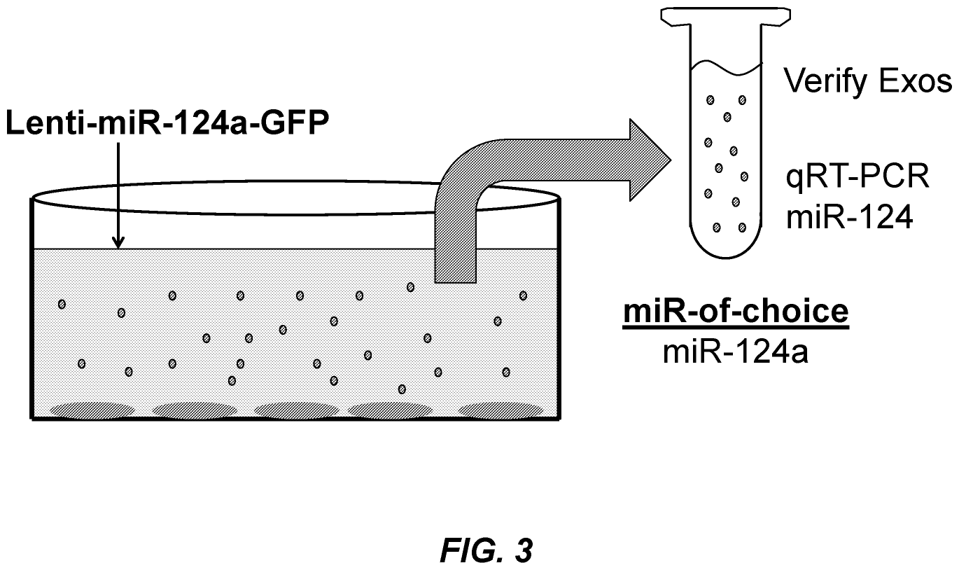

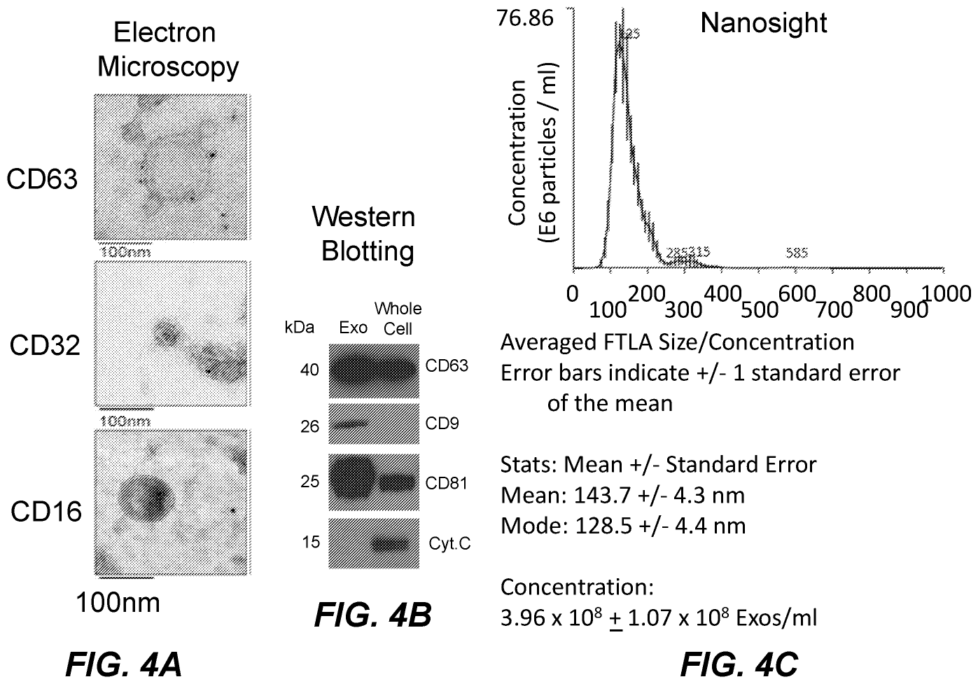

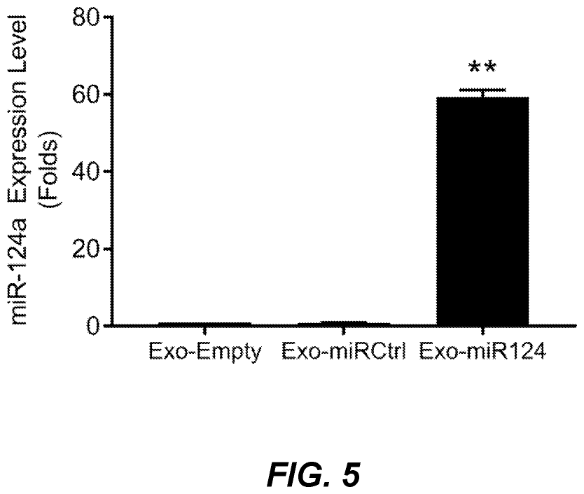

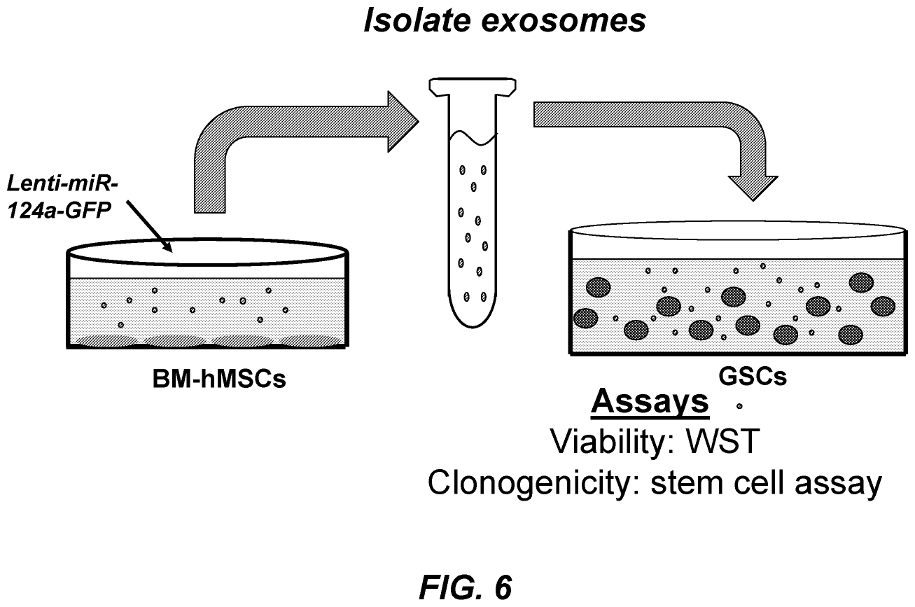

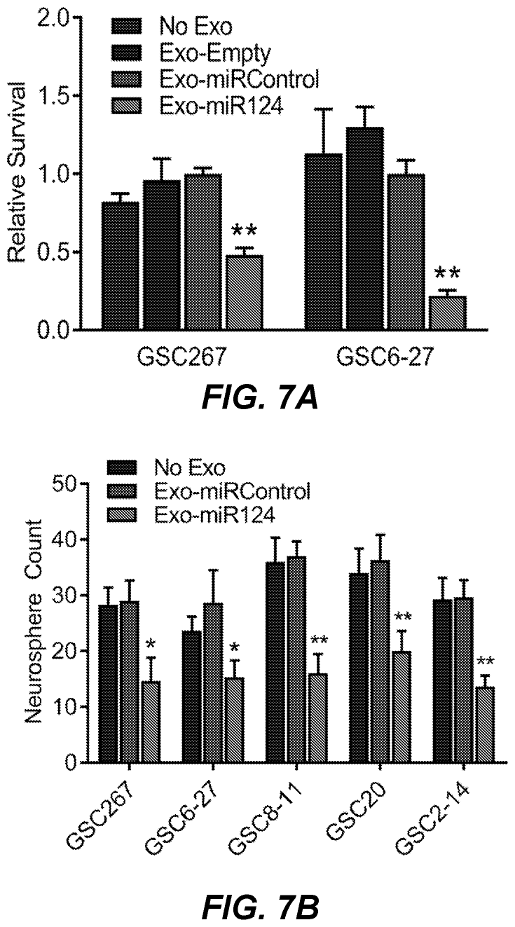

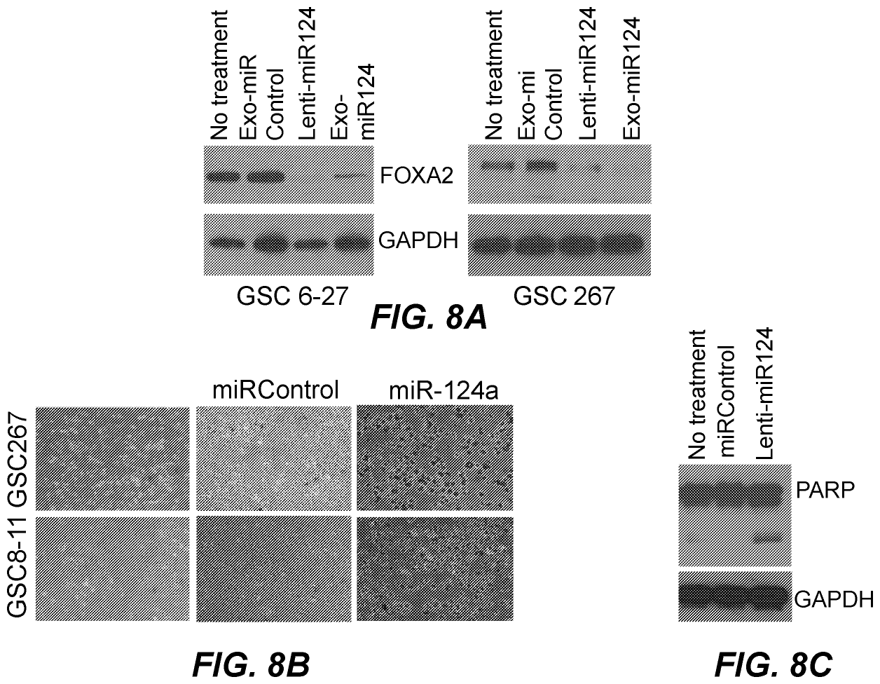

Ex vivo cultured human mesenchymal stem cells (MSCs), which secrete exosomes (nanoscale vesicles that are stable in blood) were engineered to package miR-124 into exosomes to be collected and used to systemically deliver miR-124 to GBMs. MSCs were transduced with a lentivirus containing miR-124, and exosomes (Exos-miR-124) were isolated from the supernatant (FIG. 3). Electron microscopy (FIG. 4A), western blotting (FIG. 4B), and Nanosight™ (FIG. 4C) all proved that the isolated vesicles were exosomes. qPCR revealed that miR-124a levels in Exos-miR-124 were 60-fold greater than control exosomes, which contained no miR-124a (P<0.0001) (FIG. 5). To demonstrate that Exos-miR-124 were capable of inhibiting the growth of GSCs in vitro (FIG. 6), five GSCs were treated with Exos-miR-124a, Exos-miR-Ctrl, or Exos-empty (10⁶/cell) and a significant reduction in viability (FIG. 7A) and clonogenicity (FIG. 7B) of all GSCs was shown only after treatment with Exos-miR-124 (p<0.001). In some aspects, potential mechanisms of action of Exo-miR124 show that Exos-miR-124 are capable of down regulating FoxA2, a known miR-124 target gene (FIG. 8A), induce the accumulation of lipids (FIG. 8B), and promote cell death (FIG. 8C) in GSCs treated cells.

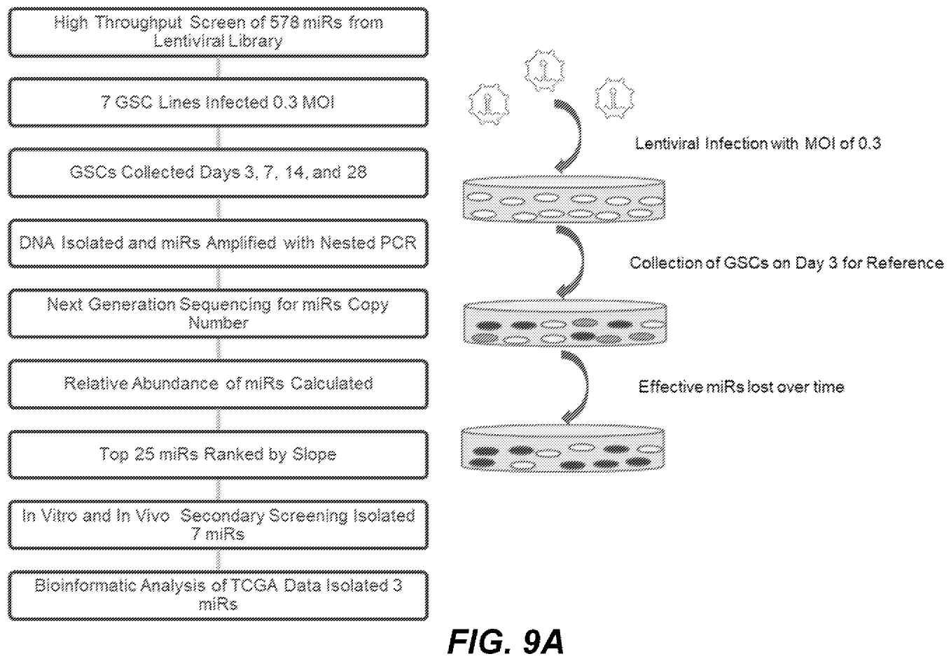

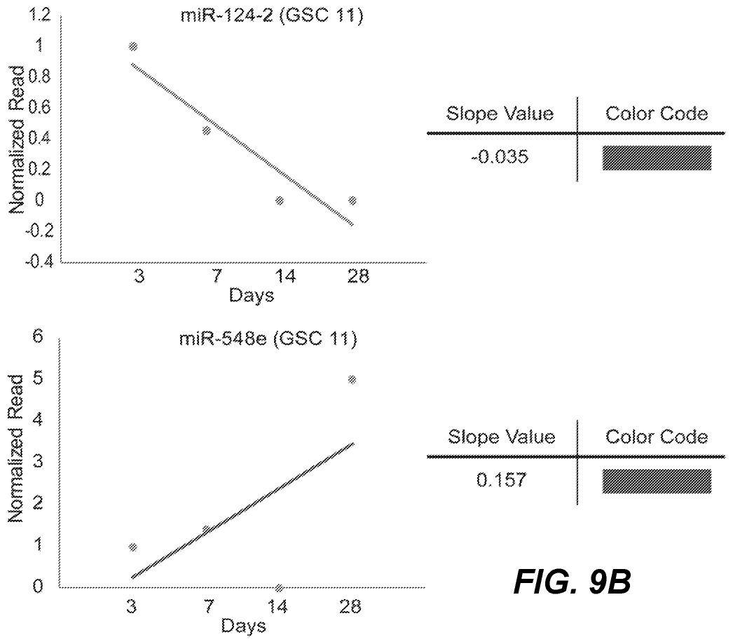

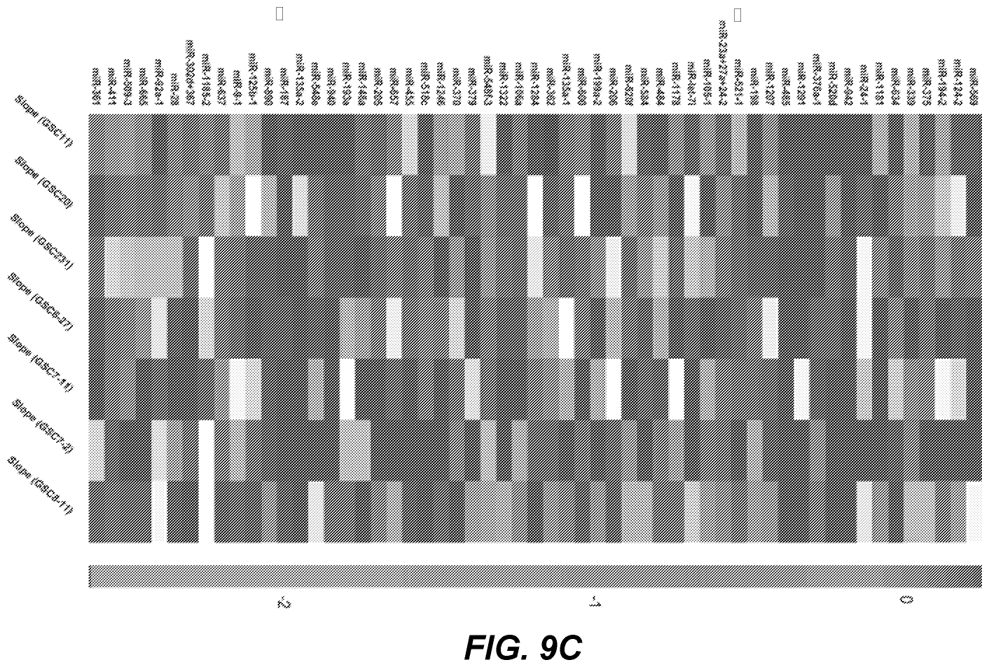

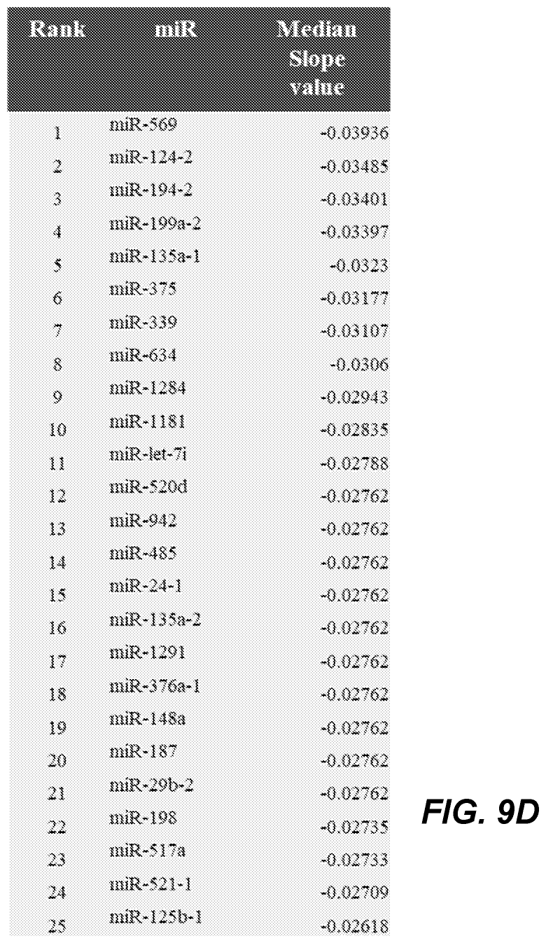

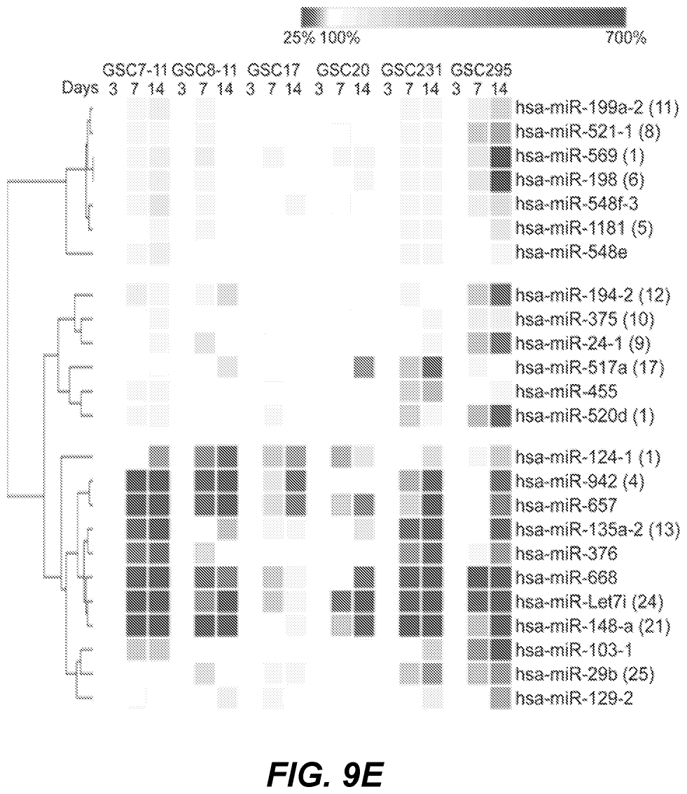

The optimal miRs for treating all classes of GBM was further defined. Specifically, an unbiased high throughput, large scale screen of miRs was conducted against a panel of seven fully annotated patient-derived patient derived GSCs. These GSCs represent all molecular sub-groups of GBM namely proneural (MDA-GSC7-11, MDA-GSC8-11), classical (MDA-GSC11, MDA-GSC7-2, MDA-GSC231, MDA-GSC6-27), and mesenchymal (MDA-GSC20). Flow chart and schematics show the key steps of the screen and data processing (FIG. 9A). The lenti-miR library used in this study contains 578 lentiviruses encoding 539 individual miRs and 39 miR clusters, resulting in a total of 603 mature miRs. Each of these GSCs was transduced with the pooled library at an MOI of 0.3 so that each cell was infected by no more than one miR. DNA was isolated from each GSC for each time point and nested PCR was performed. Next Generation Sequencing (NGS) was performed and the relative abundance of each miR was determined as the NGS read count for a specific miR on day x divided by the total number of reads for all miRs on day x. Total raw read counts across GSCs over the span of 28 days (day 3 (avg.): 1721939+177003; day 28 (avg.): 1797286±403928) remained similar (day 3 vs. day 28; p-value 0.68).

In order to rank the 603 miRs, the log₁₀ of the abundance counts was normalized to that of day 3 specific to the GSCs. This normalized count when plotted against time resulted in slope value where negative values represent depletion and positive value represent enrichment of miRs over time (days 3, 7, 14, and 28; examples of slope and color code are shown in FIG. 9B). Slope values across GSCs for each miR are shown in heat map, where miRs with consistent negative slope values across 7 GSCs are shown at the top (FIG. 9C). The miRs were ranked within each GSC according to this slope and the ranks summed up for each miR over the GSCs. This analysis resulted in a rank order with the most effective miRs across all GSCs having the lowest rank sums, of which the 25 top miRs were chosen as the most likely candidates for further study (FIG. 9D). Importantly, miR-124 came out among this group, giving confidence in the screening process.

The putative hits identified from the primary screen were then evaluated for their effectiveness in blocking GSC proliferation. Six GSCs were transduced with lentiviruses overexpressing each one of the top 25 miRs individually and their effect on GSC proliferation assessed. GSCs were transduced with a lentivirus (LV) containing precursor miR, and cell viability was determined to verify that each LV-miR was effective when tested in a non-pooled fashion. As shown in a heat map (FIG. 9E), upon hierarchical clustering, over expression of miRs-124, 148a, let7i, 135a-2, 668, 942, 657 resulted in 50-80% reduction in viability depending on the GSCs tested and are clustered together (FIG. 9D). These results showed that the top ranked individual LV-miRs were effective at decreasing GSC viability by 50% to 80%. Specifically, miRs-124, 148a, let7i, 135a-2, 668, 942, 657 had the highest inhibitory capability against all GSCs.

2. Exosomal Delivery of miRs to Brain Tumors In Vivo

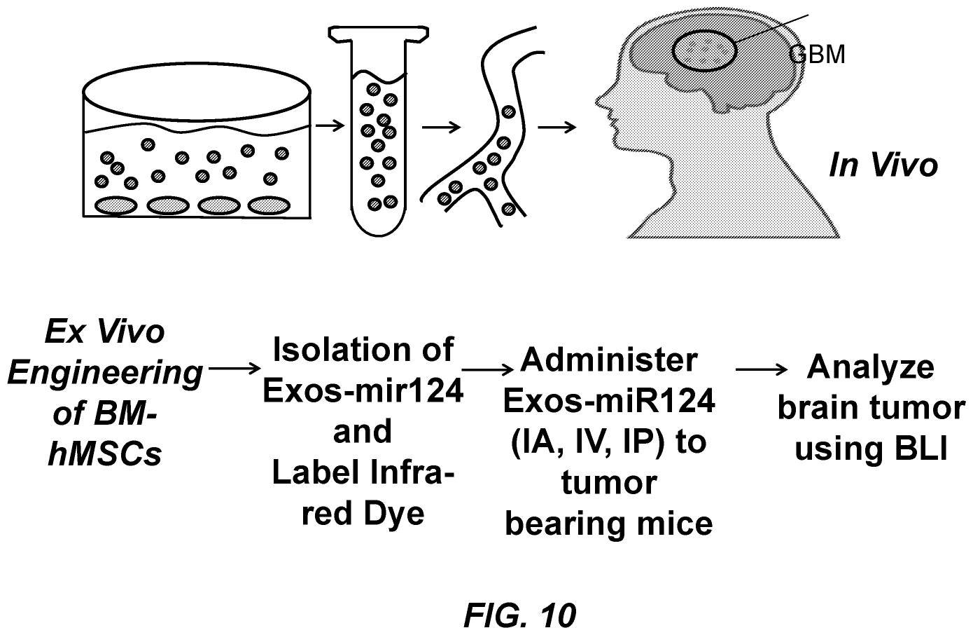

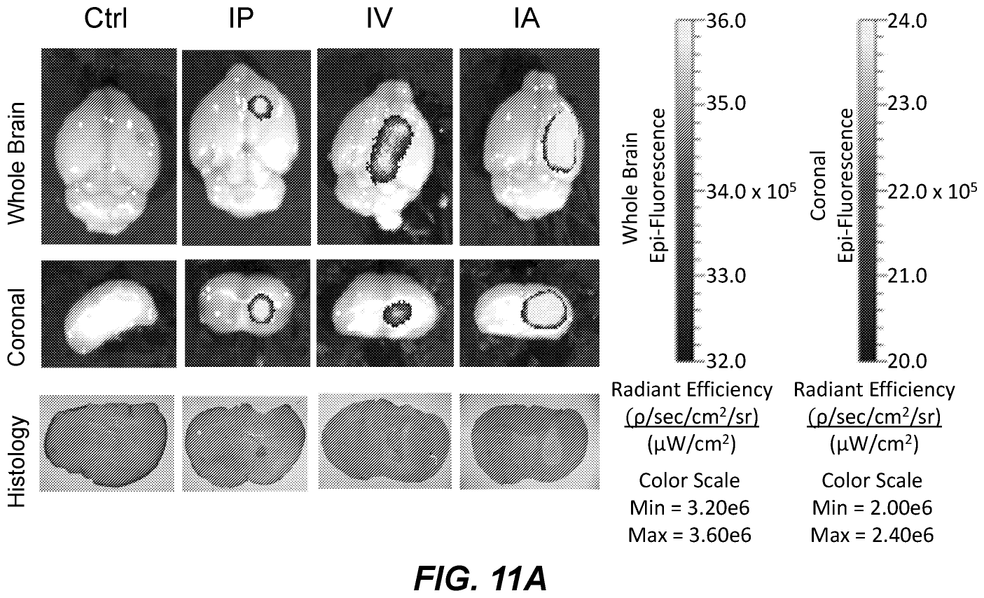

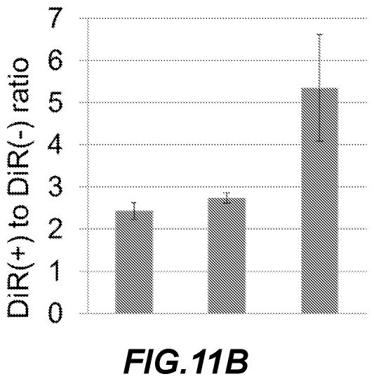

To demonstrate that exosomes are able to deliver miR-124 to brain tumors in vivo, BM-Exos were shown to have the intrinsic capacity to home to brain tumors after systemic injection. BM-Exos were isolated from BM-MSCs by ultracentrifugation and labeled with XENOLIGHT™ DiR, a near-infrared intravital dye (Caliper Life Sciences), and washed with PBS to remove free dye. These DiR-labeled exosomes (DiR-Exos, 10¹⁰ Exos/100 μl) were injected into mice harboring GSC17 or U87 orthotopic xenografts via IP, IV (tail vein), or IA (carotid artery) routes (N=3 mice/group). Brains were analyzed 8 hrs after injection using the IVIS® bioluminescence imaging (BLI) system, 200Series (Xenogen) (FIG. 10). Fluorescent images were overlaid on gray scale photographic images to allow for localization of the light source within the brain using the Live Image version 2.11 software overlay (Xenogen). Quantitative analysis showed that treatment with DiR-Exos resulted in high signal within the tumor with the intraarterial (IA) route, with intraperitoneal (IP) and intravenous (IV) injection also yielding effective homing (FIG. 11). These data indicate that, in some aspects, MSC-Exos are capable of homing to brain tumors overcoming the BBB/BTB, and suggests that, in some aspects, there may be advantages to IA delivery in terms of reducing first-pass losses, but IV injection was also effective (FIGS. 11A, 11B).

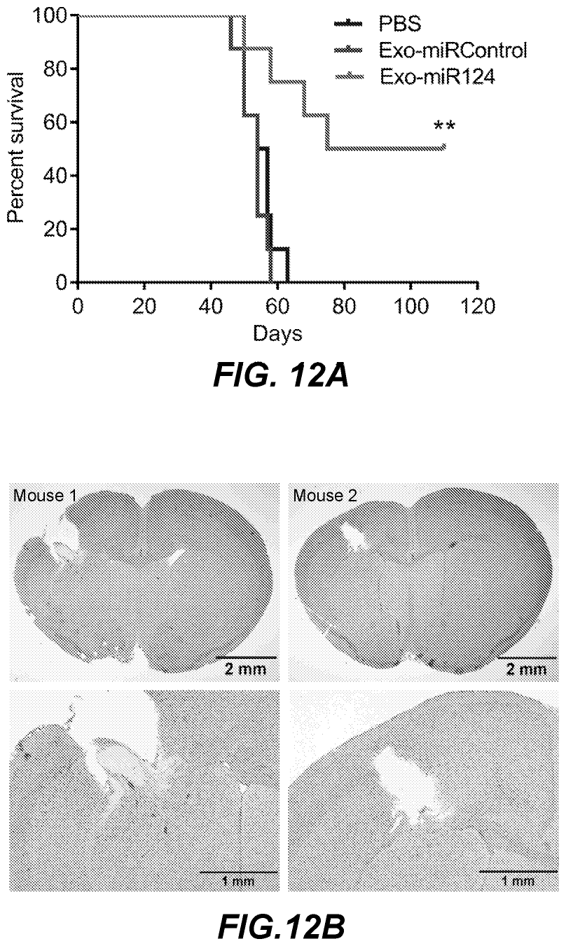

To assess efficacy of Exos-miR-124 in vivo, GSC267 was implanted into the frontal lobe of nude mice (N=8/group), and after 7 days animals, were treated with BM-Exo-miR-124, BM-Exo-miR-control, or PBS by IP injection (as a surrogate for IV injection) every other day (10¹⁰ Exo/100 μl). Exosomes were also injected IA on days 14 and 21. Whereas all controls were dead by 60 days after tumor implantation (median survival BM-Exo-miR-control: 54 days; PBS: 55 days), 50% of animals treated with BM-Exo-miR-124 were alive at 110 days (median: 79 days, P=0.009) (FIG. 12A). Histological analyses of surviving mice showed eradication of the tumors, indicating that systemic delivery of Exo-miR-124 is efficacious in vivo (FIG. 12B). Taken together, these data indicate that, in some aspects, miR-124 is a highly effective anti-GBM therapeutic and that, in some aspects, BM-MSC-derived exosomes can be used to systemically delivery miR-124a to achieve cures of intracranial GBMs.

### 3. Preparation and Characterization of Umbilical Cord Mesenchymal Stem Cell-Derived Exosomes

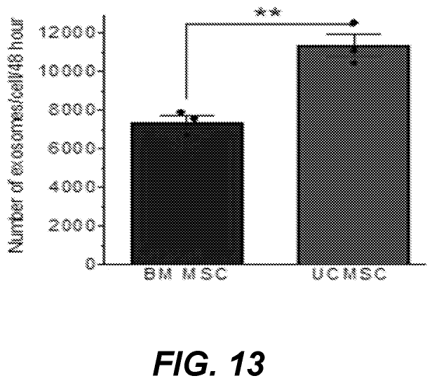

Although exosomes were originally isolated from BM-MSCs, the supply of BM-MSCs can be limited, and extraction of bone marrow from normal donors can be expensive. In contrast, in some aspects, readily available umbilical cord tissue can be extremely attractive and low-cost alternative source of MSCs (UC-MSCs). Importantly, UC-MSCs were shown to exhibit a higher expansion capacity and a greater exosome yield per cell than BM-MSCs (FIG. 13). Given the advantages of UC-MSCs over BM-MSCs as a source of exosomes, UC-Exos can be used, in some aspects, as delivery vehicles in brain tumor therapy, while in other aspects related to selected diseases and situations, BM exosomes can be used.

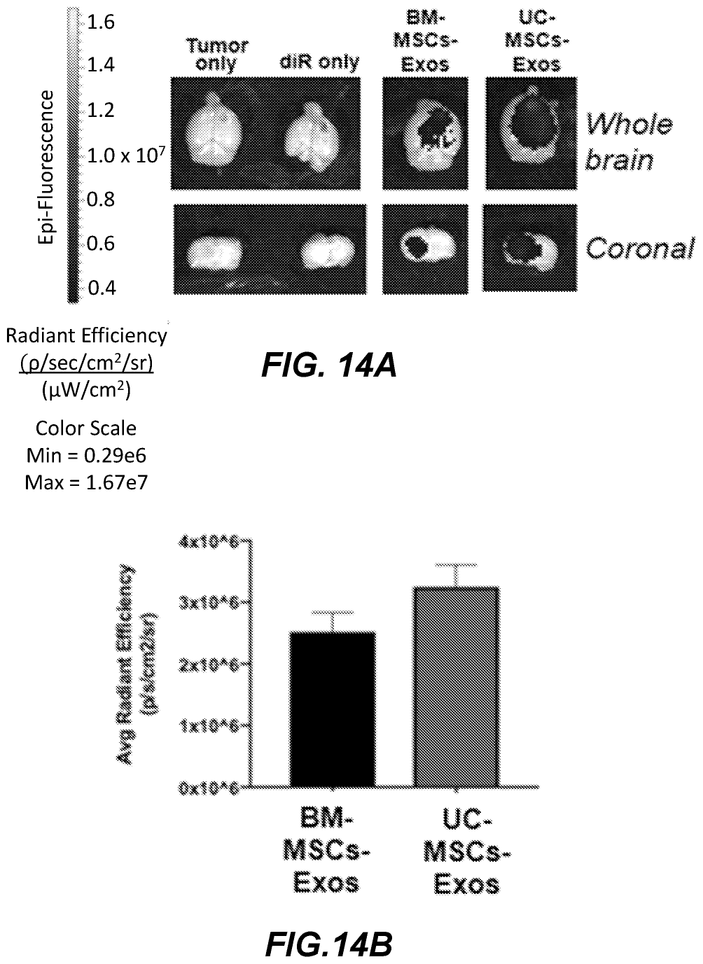

UC-Exos, like BM-Exos, were shown to home to brain tumors in vivo. Specifically, mice were implanted with U87 cells and after 7 days, DiR-labeled BM-Exos or UC-Exos (10¹⁰ exosomes/100 ul) were delivered via IA injection (n=3/group). After 24 hrs, exosome-homing was assessed by BLI. Compared with controls (no injection, injection of DiR only), fluorescent-labeled UC-Exos were detected in the tumors but not in the surrounding normal brain in 3/3 mice. Quantitative analyses demonstrated statically significantly higher levels of fluorescence in U87 tumors treated with UC-Exos compared with treatment with BM-Exos, supporting the application, in some aspects, of UC-Exos in the clinical setting (FIGS. 14A-14B).

4. Electroporation of Exosomes to Load miRs

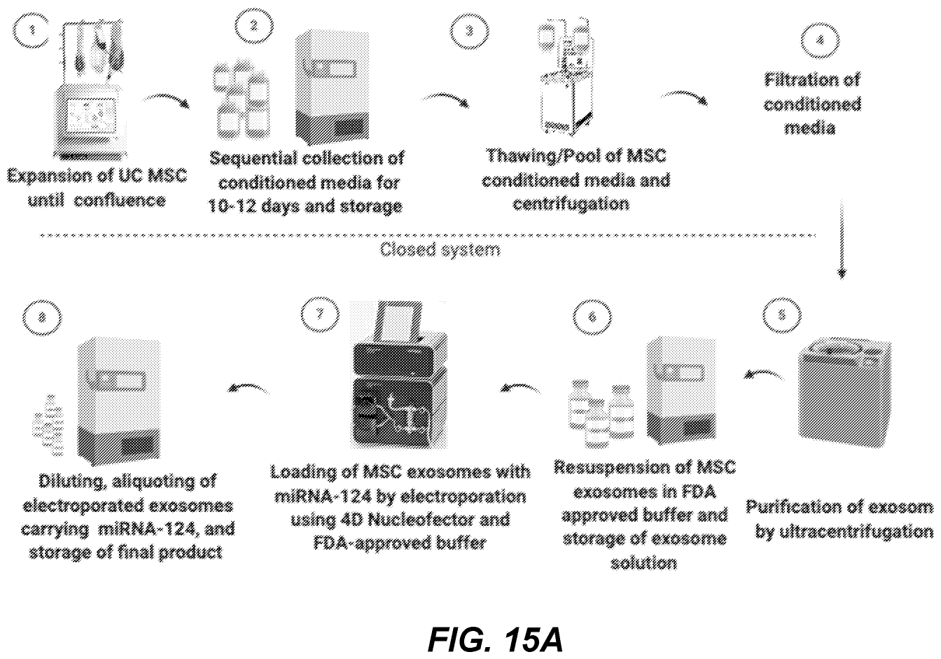

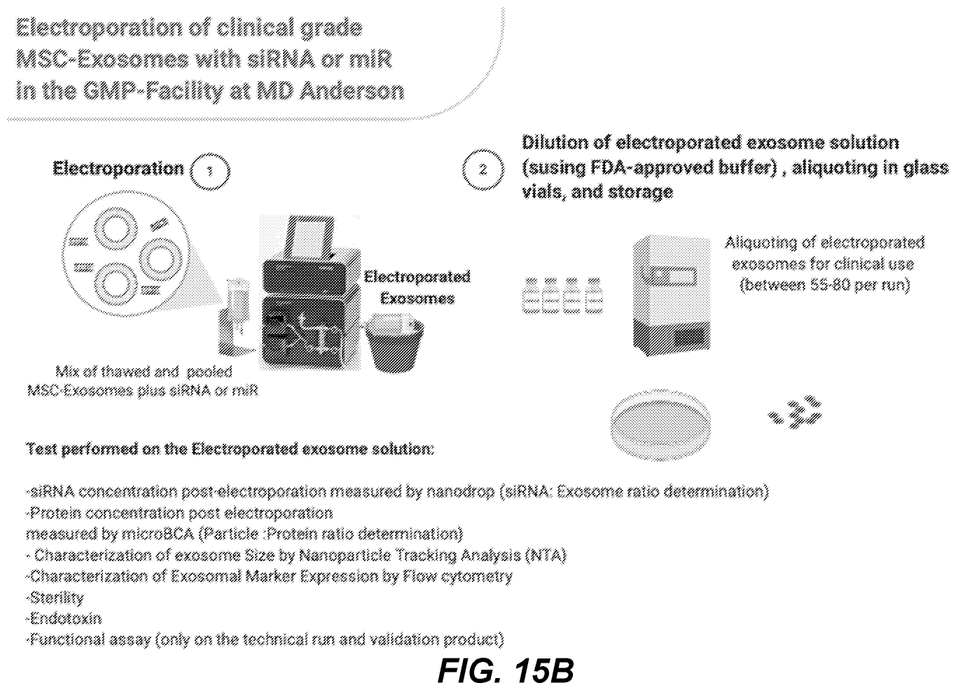

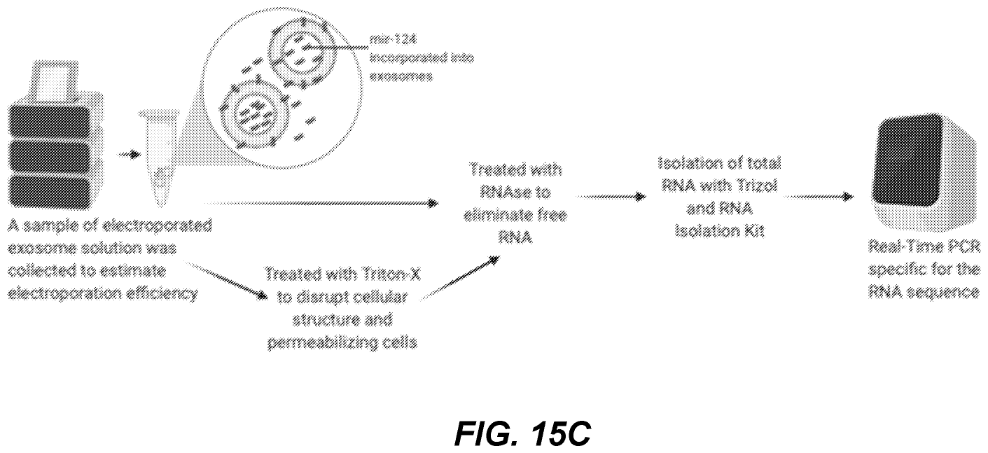

Of equal importance for clinical development is the method of loading miR-124 into exosomes. In some cases, a strategy for producing Exo-miR-124 relies on transducing MSCs with lentivirus (LV) containing the cDNA of miR-124, followed by isolation of the Exo-miR-124 from the supernatant. In other cases, mature miR-124 mimics are directly loaded into exosomes by electroporation (FIGS. 15A, 15B, and 15C).

Standard operating procedures (SOPs) were developed for the electroporation of miR-124 into UC-Exos which are GMP-compliant and can be thawed and infused directly into patients as the infusion reagents are FDA approved for clinical use. Specifically, UC-MSCs were cultured, supernatant collected, and UC-Exos isolated by centrifugation. Human miR-124 double stranded mature miR-mimic (Sigma Aldrich) was electroporated into UC-Exos. The initial optimization experiments included 16 reactions testing 15 different nucleofector programs plus one control (non-electroporated) performed in the 16-well nucleocuvette strip. Each electroporation reaction contained approximately 1 μg of total exosomal protein (measured by microBCA and equal to 1×10⁸ UC-Exos determined by NANOSIGHT™) and 0.5 μg miR-124 (measured by NANODROP™) mixed in 20 μl GMP-level buffer (Plasma-Lyte A). These exosomes were electroporated using a 4D-NUCLEOFECTOR® system (Lonza), producing Exo-miR-124.

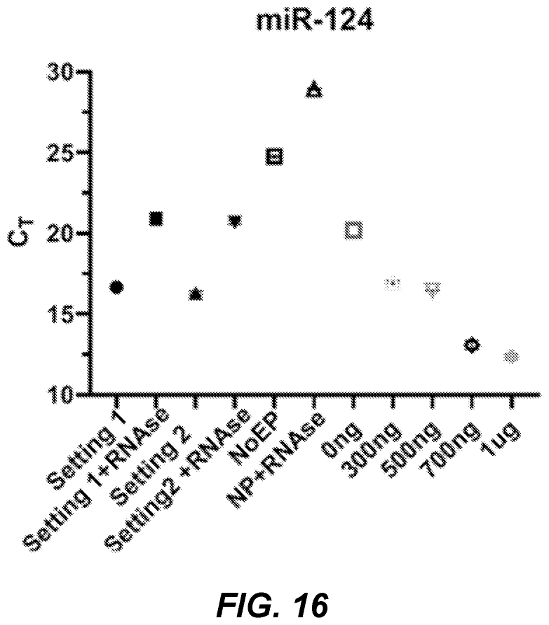

To assess the amount of miR-124 μloaded into the UC-Exos, Exo-miR-124 was treated with RNase (or without RNase as control) to eliminate any free miR-124, total RNA was isolated using TRIZOL™, and RT-qPCR was performed using primers specific for miR-124. Samples with known quantities of miR-124 were simultaneously assayed to develop a standard curve. Based on the results, two electroporation programs that consistently had the lowest Ct value across all replicates were identified. Subsequently, results from the initial optimization experiments using the two preselected programs were confirmed using 100 μl cuvette in which 2 μg of total exosome protein (2×10⁸ UC-Exos) and 1.0 μg miR-124 were mixed in 100 μl GMP-level buffer (Plasma-Lyte A), and RNA content was again assayed by RT-qPCR (FIG. 16). Exposure to RNase revealed that encapsulation within exosomes protected the miR-124 from degradation (FIG. 16). Extrapolation from the standard, or normal, curve indicated that 2 μg exosome protein (equal to 2×10⁸ UC-Exos) contained ˜350 ng of miR-124 (i.e., 35% of the 1 ug miR-124 is loaded into exosomes). This translates into 1.75×10⁻³ pg miR-124/UC-Exo or 7.6×10⁴ miR-124 molecules/UC-Exo (molecular weight miR-124=13,857M). Taken together, these studies demonstrate that, in some aspects, UC-Exos containing ample miR-124 can be successfully produced using this electroporation strategy.

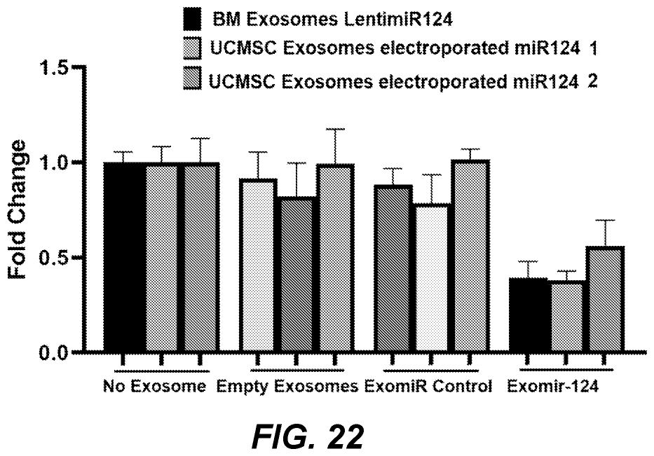

As shown in FIG. 22, treatment with exomiR-124 significantly reduced the number GSC267 glioblastoma stem cells. Effect of treatment with BM-MSC-Exosomes (BM-MSC-Exos) and two samples of UC-MSC-Exosomes (UC-MSC-Exos) were compared to exosomes alone (Empty Exosomes), MSC-Exos containing control miRNA (ExomiR control), and MSC-Exos containing miR124a (miR-124) via lentivirus (BM-MSC-Exos) or electroporation (UC-MSC-Exos) on the viability of the GSC267 glioblastoma cell line was assayed by counting viable cells using trypan blue exclusion.

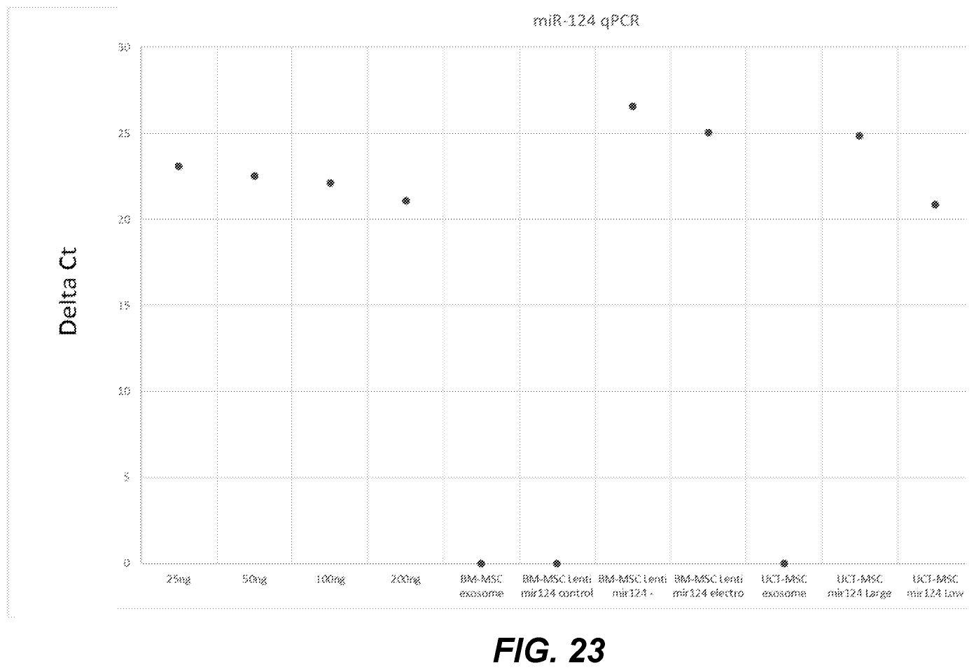

As shown in FIG. 23, a higher copy number of miR124 was loaded into cord tissue exosomes. RT-qPCR results were obtained for miR-124 levels on BM-MSC-Exosomes produced by lentivirus and UC-MSC-Exosomes after electroporation using a small or large scale electroporation process, with the appropriate controls. miRNA concentrations from 25ng to 200ng were used to obtain a standard curve (right half of graph). The cord tissue exosomes had the best results in terms of the lower delta Ct indicating a higher copy number of miR124 in the exosomes.

5. Delivery of Electroporated Exosomes Loaded with miR to Glioma Stem Cells In Vitro

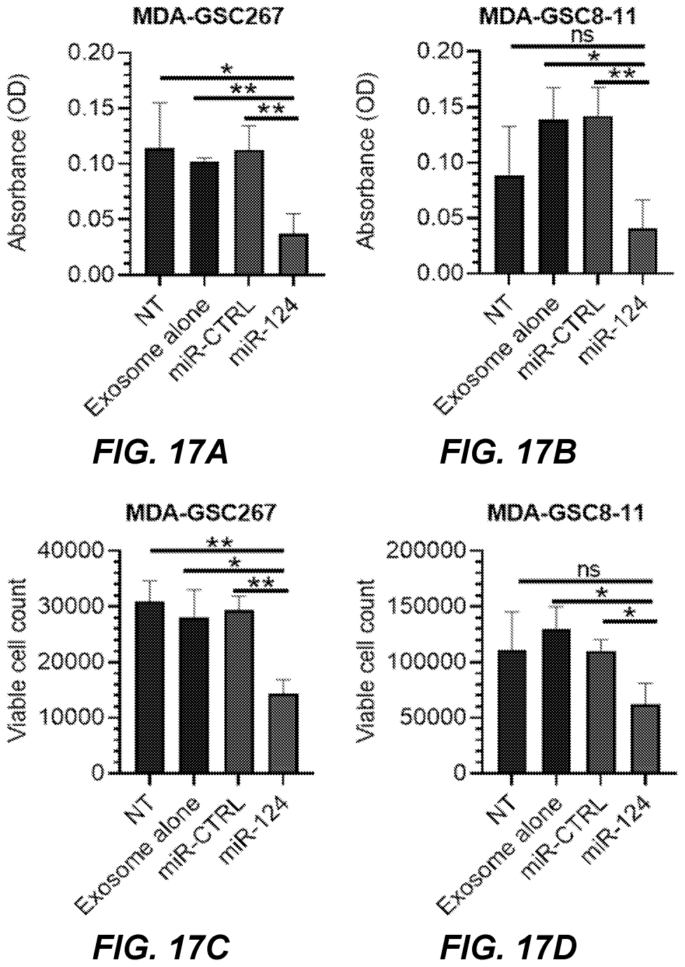

To demonstrate that the electroporation strategy resulted in efficacious Exo-miR-124, experiments were conducted to confirm that, like BM-Exo-miR-124, Exo-miR-124 produced by electroporation of UC-Exos with mature miR-124 mimics were capable of inhibiting the growth of human gliomas. Specifically, UC-Exos were isolated from the supernatant of ex vivo cultured UC-MSCs grown in exosome-free medium under GMP conditions. Production was scaled up by mixing 80 μg UC-Exos (80×10⁸ Exos) with 40 μg of mature miR-124 mimic (Sigma-Aldrich) (2:1 ratio) and electroporated using 1 ml cuvettes in FDA-approved buffer using the 4D-NUCLEOFECTOR® LV system (Lonza). Exosomes were then frozen down at −80° C. until their use. To test efficacy, GSC-267 and GSC8-11 (2.5×10³/well) were treated with Exo-miR-124, Exo-miR-scrambled (2×10⁸ loaded exosomes per well; 350 ng or 50 nM miR-scramble or miR-124), UC-Exos alone (2×10⁸/well), or PBS for 3 days and assayed cell viability after 1 week (FIGS. 17A-17D), mimicking experiments with BM-Exo-miR-124. Treatment of both GSCs resulted in 60-80% reduction in cell viability (p<0.01, FIG. 17A-17D), indicating that, in some aspects, Exo-miR-124 produced from UC-Exos by electroporation is efficacious and the production method is effective. 6. Electroporation of exosomes to load siRNAs

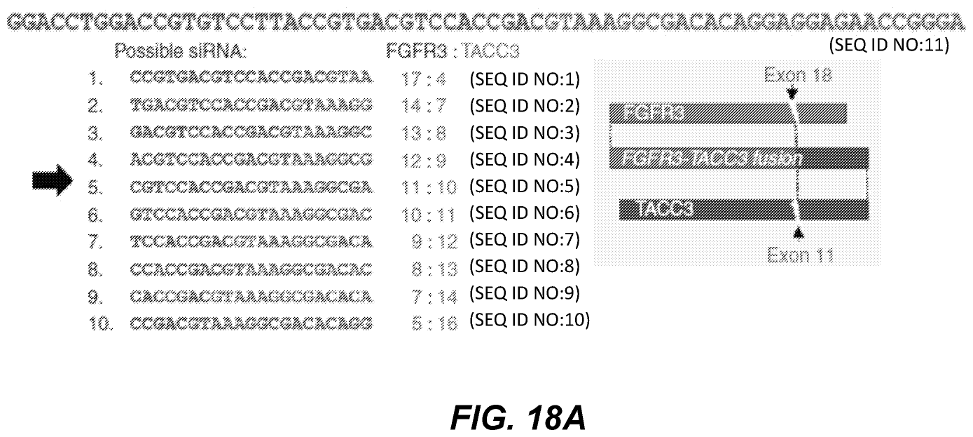

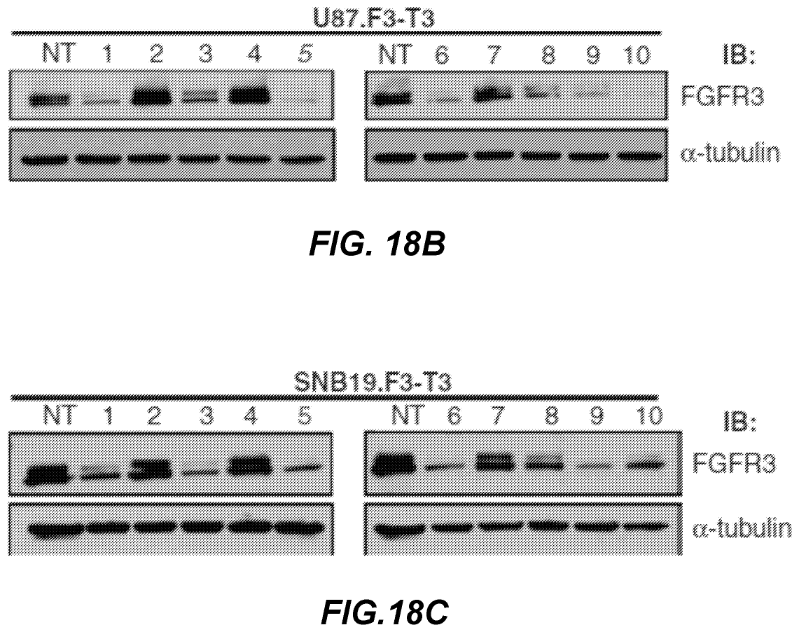

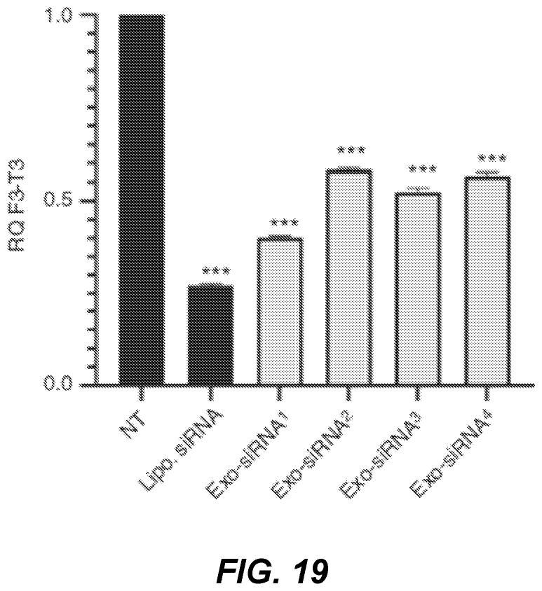

In addition to loading exosomes with miRs, synthetic interfering RNAs (siRNA) can also be loaded into MSC-derived exosomes. siRNA against the FGFR3-TACC3 fusion was developed and loaded into exosomes. The FGFR3-TACC3 fusion (F3-T3) has been shown to drive gliomagenesis. In some aspects, depleting F3-T3 using custom siRNA to the fusion breakpoint can result in successful inhibition of F3-T3+ GBMs. F3-T3 was overexpressed in U87 and SNB19 lines, and two F3-T3+ glioma stem-like cells (GSC13 and 231) were identified. 10 unique siRNAs (iF3T3) were engineered that specifically spanned the most common F3-T3 breakpoint with varying degrees of overlap, and 7/10 of these siRNA were demonstrated to reduce expression of F3-T3 (FIGS. 18A-18C). Importantly, these two fusion junction siRNAs did not deplete wild-type FGFR3 or TACC3. iF3T3 decreased cell viability in F3T3+ GBM cell lines. UC-MSC exosomes successfully delivered iF3T3 in vitro, resulting in F3-T3 depletion (FIG. 19). These studies show that, in some aspects, UC-MSC exosomes may be a plausible vehicle to deliver iF3T3.

### 7. Administration of Umbilical Cord Mesenchymal Stem Cell-Derived Exosomes to Treat Radiation and Chemotherapy-Induced Toxicities

In addition to the capacity of UC-Exos to deliver therapeutic agents to gliomas, the potential of UC-MSCs to mitigate treatment induced CNS toxicities was also demonstrated. Based on recent evidence indicating that exosomes are capable of reversing traumatic brain injury and inflammation, in some aspects, treatment with MSC-derived exosomes may be effective as effective as MSCs at reversing chemoradiation-induced brain injury. A robust preclinical model of chemoradiation-induced brain injury was established in C57BL/6 mice. Specifically, cranial irradiation was delivered to the whole mouse brain to a total dose of 20 Gy (2 Gy/fraction x 10 fractions) using a Precision X-ray Xrad225 small animal irradiator with image guidance to allow daily fractionation with reproducible dosimetry. TMZ was given by oral gavage 2 hours prior to cranial irradiation at a dose of 33 mg/kg. After one month animals were subjected to a series of validated behavioral tests: (1) novel object place recognition and Y-maze for assessing learning and memory, (2) puzzle box to measure executive function, and (3) the elevated zero maze, forced swim, and open field test to assess mood and motor ability. These analyses consistently showed that chemoradiation resulted in decreased cognitive functions across behavioral paradigms without adverse effects on mood or motor function. Executive function was assessed using the puzzle box described by G. S. Chiu et al. Oncotarget. 2018 Oct. 30; 9(85):35581-97, and J. Ma et al., Acta Neuropathol Commun. 2018 Oct. 1; 6(1):103.

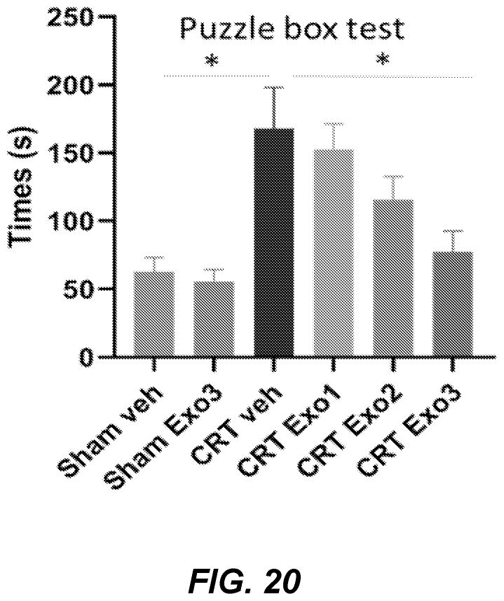

Using this model, proof-of-concept experiments revealed that IV administration of UC-Exos (dose: Exo1=0.8×10⁹/150 μl, Exo2=8.8×10⁹/150 μl, Exo3=1.7×10¹⁰/150 μl) given 2 and 4 days after chemoradiation, resulted in dose-dependent reversal of cognitive dysfunction (Puzzle box test) 1 month after chemoradiation with 1.7×10¹⁰ exosomes/150 μl improving cognitive function to 80% of controls (FIG. 20). These data suggested that, in some embodiments, UC-Exos may be effective in the treatment of neurocognitive toxicities secondary to radiation and chemotherapy.

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method of producing therapeutic exosomes comprising the steps of:
(a) culturing mesenchymal stem cells (MSCs); and
(b) collecting the exosomes from the culture;
wherein, prior to step (a), the MSCs are transfected or transduced to load one or more therapeutic agents into the MSCs, and
wherein the exosomes are generated from the transfected or transduced MSCs and comprise the one or more therapeutic agents.

2. A method of producing therapeutic exosomes comprising the steps of:
(a) culturing mesenchymal stem cells (MSCs);
(b) collecting the exosomes from the culture; and
(c) electroporating the collected exosomes to load one or more therapeutic agents into the exosomes.

3. The method of claim 1 or claim 2, wherein the culturing step (a) occurs in the presence of specific concentrations or conditions of CO₂, O₂, and nitrogen.

4. The method of claim 3, wherein the concentration of CO₂ is 5%.

5. The method of claim 3 or claim 4, wherein the concentration of O₂ is 20%.

6. The method of any one of claims 1-5, wherein the culturing step (a) occurs under conditions balanced with nitrogen.

7. The method of any one of claims 1-6, wherein the MSCs are from umbilical cord tissue, bone marrow, adipose tissue, dental tissue, placental tissue, or a mixture thereof.

8. The method of any one of claims 1-7, wherein the MSCs are from umbilical cord tissue.

9. The method of any one of claims 1-8, wherein the method occurs in an automated system.

10. The method of claim 9, wherein system is configured to comprise continuous perfusion of medium through at least part of the system.

11. The method of claim 9 or 10, wherein the system is closed or semi-closed.

12. The method of any one of claims 1-11, wherein the method occurs in a bioreactor.

13. The method of claim 12, wherein the bioreactor comprises multiple hollow fibers.

14. The method of claim 13, wherein one or more surfaces inside the bioreactor are modified to allow adherence of cells.

15. The method of claim 14, wherein the one or more surfaces inside the bioreactor are modified to comprise one or more extracellular matrix proteins.

16. The method of claim 15, wherein the extracellular matrix protein is fibronectin.

17. The method of any one of claims 9-16, further comprising the step of extracting a sample from the system.

18. The method of claim 17, wherein the sample is tested for one or more characteristics of the exosomes.

19. The method of any one of claims 1-18, wherein step (b) utilizes media that lacks platelet lysate.

20. The method of any one of claims 1-19, wherein step (b) utilizes media that comprises L-alanyl-L-glutamine dipeptide.

21. The method of any one of claims 1-20, wherein the culturing step (a) utilizes media that comprises L-alanyl-L-glutamine dipeptide.

22. The method of any one of claims 1-21, wherein the culturing step (a) utilizes alpha MEM media, heparin, human platelet lysate, and L-alanyl-L-glutamine dipeptide.

23. The method of any one of claims 1-22, wherein steps (a) and (b) occur more than once.

24. The method of any one of claims 1-23, wherein steps (a) and (b) occur 2, 3, 4, 5, 6, 7, 8, 9, 10, or more times.

25. The method of any one of claims 1-24, wherein step (b) occurs more than once and the collecting occurs in intervals of about 48 hours.

26. The method of any one of claims 2-25, wherein the collected exosomes are suspended in a sterile, isotonic, non-pyrogenic buffer prior to electroporation in step (c).

27. The method of claim 26, wherein the buffer comprises Plasma-Lyte A.

28. The method of any one of claims 2-27, wherein about 1×10⁸ to about 10×10¹² collected exosomes are electroporated in step (c).

29. The method of any one of claims 1-28, wherein the one or more therapeutic agents is miRNA, siRNA, shRNA, protein, peptides, drug, lipids, DNA, RNA, or a combination thereof.

30. The method of claim 29, wherein the one or more therapeutic agents is protein, peptides, drugs, and/or lipids, and wherein the concentration of the protein, peptides, drugs, and/or lipids is between 1 μg/mL and 1000 mg/mL.

31. The method of claim 29 or claim 30, wherein the protein comprises an antibody or antibody fragment.

32. The method of any one of claims 29-31, wherein the one or more therapeutic agents is miRNA, and wherein the concentration of miRNA is between 1 μg/mL and 200 μg/mL.

33. The method of any one of claims 29-32, wherein the miRNA comprises miR-124, miR-148a, miR-let7i, miR-135a-2, miR-668, miR-942, miR-657.

34. The method of any one of claims 29-33, wherein the one or more therapeutic agents is siRNA, shRNA, and/or RNA, and wherein the concentration of siRNA, shRNA, and/or RNA is between 1 μg/mL and 200 μg/mL.

35. The method of any one of claims 29-34, wherein the siRNA comprises siRNA against the fusion breakpoint of a FGFR3-TACC3 gene fusion product.

36. The method of any one of claims 29-35, wherein the DNA comprises at most 1000 base pairs.

37. The method of any one of claims 29-36, wherein the concentration of DNA is between 1 μg/mL and 200 μg/mL.

38. The method of any one of claims 1-37, wherein the loading efficiency of the one or more therapeutic agents is at least 30%, at least 40%, at least 50%, at least 60%, at least 70%, at least 80%, or at least 90%.

39. The method of any one of claims 1-38, further comprising the step of delivering an effective amount of the exosomes to an individual in need thereof.

40. The method of claim 39, wherein following delivery to an individual in need thereof, the exosomes provide neuroprotection against central nervous system toxicities induced by one or more additional therapies delivered to the individual.

41. The method of claim 39 or claim 40, wherein following delivery to an individual in need thereof, the exosomes ameliorate or reverse cognitive dysfunction and/or neurodegeneration induced by one or more additional therapies delivered to the individual.

42. The method of claim 40 or claim 41, wherein the one or more additional therapies delivered to the individual comprise chemotherapy, radiotherapy, or a combination thereof.

43. The method of any one of claims 39-42, wherein following delivery to an individual in need thereof, the exosomes reduce inflammation.

44. The method of any one of claims 39-43, wherein following delivery to an individual in need thereof, the exosomes provide a tissue-regenerative and/or a tissue-reparative effect to tissue in need of regeneration and/or repair.

45. The method of claim 44, wherein the tissue is in need of regeneration and/or repair due to toxicities induced by one or more additional therapies delivered to the individual.

46. The method of claim 44 or claim 45, wherein the one or more additional therapies delivered to the individual comprise chemotherapy, radiotherapy, or a combination thereof.

47. The method of any one of claims 44-46, wherein the tissue is in need of regeneration and/or repair due to inflammation, contusions, sprains, tendonitis, bursitis, stress injuries, strains, or a combination thereof.

48. The method of any one of claims 44-47, wherein the tissue in need of regeneration and/or repair comprises soft tissue, brain, lung, spleen, liver, heart, kidney, pancreas, intestine, testis, or bone.

49. The method of any one of claims 39-48, wherein following delivery to an individual in need thereof, the exosomes provide a skin-regenerative and/or a skin-reparative effect to skin in need of regeneration and/or repair.

50. The method of claim 49, wherein the skin is in need of regeneration and/or repair due to toxicities induced by one or more additional therapies delivered to the individual.

51. The method of claim 49 or claim 50, wherein the one or more additional therapies delivered to the individual comprise chemotherapy, radiotherapy, or a combination thereof.

52. The method of any one of claims 49-51, wherein the skin is in need of regeneration and/or repair due to a skin disorder.

53. The method of any one of claims 49-52, wherein the skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

54. The method of any one of claims 39-53, wherein following delivery to an individual in need thereof, the exosomes provide a wound healing effect to tissue or skin in need of wound healing.

55. The method of claim 54, wherein the tissue or skin is in need of wound healing due to toxicities induced by one or more additional therapies delivered to the individual.

56. The method of claim 54 or claim 55, wherein the one or more additional therapies delivered to the individual comprise chemotherapy, radiotherapy, or a combination thereof.

57. The method of any one of claims 54-56, wherein the tissue or skin is in need of wound healing due to a tissue injury or skin disorder.

58. The method of any one of claims 54-57, wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

59. The method of any one of claims 39-58, wherein the exosomes cross the blood-brain barrier, the blood-tumor barrier, or a combination thereof.

60. The method of any one of claims 39-59, wherein the exosomes directly or indirectly treat an immune disorder, cancer, heart disease, kidney disease, lung disease, liver disease, infection, tissue injury, skin disorder, wound, or a combination thereof, or one or more symptoms of an immune disorder, cancer, heart disease, kidney disease, lung disease, liver disease, infection, tissue injury, skin disorder, wound, or a combination thereof, in an individual in need thereof having an immune disorder, cancer, heart disease, kidney disease, lung disease, liver disease, infection, tissue injury, skin disorder, wound, or a combination thereof.

61. The method of claim 60, wherein the one or more symptoms comprise central nervous system toxicities, cognitive dysfunction, neurodegeneration, inflammation, tissue degeneration, tissue damage, skin damage, wounds, trauma, burns, or a combination thereof.

62. The method of claim 60 or claim 61, wherein the immune disorder is an autoimmune disorder or an alloimmune disorder.

63. The method of claim 62, wherein the immune disorder is graft-versus-host disease.

64. The method of claim 60 or claim 61, wherein the cancer is a solid tumor cancer.

65. The method of claim 64, wherein the cancer is a CNS cancer or a CNS-related cancer.

66. The method of claim 64 or claim 65, wherein the cancer is glioblastoma.

67. The method of claim 60 or claim 61, wherein the tissue injury is inflammation, contusions, sprains, tendonitis, bursitis, stress injuries, strains, or a combination thereof.

68. The method of claim 60 or claim 61, wherein the skin disorder is inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

69. The method of claim 60 or claim 61, wherein the wound is a result of a tissue injury or skin disorder, and wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

70. Exosomes produced from any one of the methods of claims 1-37.

71. A composition comprising the exosomes of claim 70.

72. A pharmaceutical composition comprising the exosomes of claim 70.

73. The pharmaceutical composition of claim 72, further comprising one or more additional therapeutic agents.

74. A method of treating an individual for an immune disorder, cancer, heart disease, kidney disease, lung disease, liver disease, infection, tissue injury, skin disorder, wound, or a combination thereof, comprising the step of administering to the individual a therapeutically effective amount of exosomes produced by the method of any one of claims 1-37, the composition of claim 71, or the pharmaceutical composition of claims 72-73.

75. The method of claim 74, wherein the cancer is a solid tumor cancer.

76. The method of claim 75, wherein the cancer is a CNS cancer or a CNS-related cancer.

77. The method of claim 75 or claim 76, wherein the cancer is glioblastoma.

78. The method of claim 74, wherein the immune disorder is an alloimmune disorder or an autoimmune disorder.

79. The method of claim 78, wherein the immune disorder is graft-versus-host disease.

80. The method of claim 74, wherein the tissue injury is inflammation, contusions, sprains, tendonitis, bursitis, stress injuries, strains, or a combination thereof.

81. The method of claim 74, wherein the skin disorder is inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof.

82. The method of claim 74, wherein the wound is a result of a tissue injury or skin disorder, and wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof.

83. The method of any one of claims 74-82, further comprising administering to the individual a second therapy for the respective immune disorder, cancer, heart disease, kidney disease, lung disease, liver disease, infection, tissue injury, skin disorder, wound, or a combination thereof.

84. A method of protecting against central nervous system toxicities in an individual having an immune disorder, cancer, heart disease, kidney disease, lung disease, liver disease, infection, tissue injury, skin disorder, wound, or a combination thereof, wherein the central nervous system toxicities are induced by one or more therapies delivered to the individual for treatment of the immune disorder, cancer, heart disease, kidney disease, lung disease, liver disease, infection, or a combination thereof, the method comprising the step of administering to the individual a therapeutically effective amount of exosomes produced by the method of any one of claims 1-37, the composition of claim 71, or the pharmaceutical composition of claims 72-73.

85. A method of ameliorating or reversing cognitive dysfunction and/or neurodegeneration in an individual having an immune disorder, cancer, heart disease, kidney disease, lung disease, liver disease, infection, tissue injury, skin disorder, wound, or a combination thereof, wherein the cognitive dysfunction and/or neurodegeneration is induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of any one of claims 1-37, the composition of claim 71, or the pharmaceutical composition of claims 72-73.

86. A method of regenerating and/or repairing tissue in need thereof in an individual having an immune disorder, cancer, heart disease, kidney disease, lung disease, liver disease, infection, tissue injury, skin disorder, wound, or a combination thereof, wherein the tissue is in need of regeneration and/or reparation due to toxicities induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of any one of claims 1-37, the composition of claim 71, or the pharmaceutical composition of claims 72-73.

87. A method of regenerating and/or repairing skin in need thereof in an individual having an immune disorder, cancer, heart disease, kidney disease, lung disease, liver disease, infection, tissue injury, skin disorder, wound, or a combination thereof, wherein the skin is in need of regeneration and/or reparation due to toxicities induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of any one of claims 1-37, the composition of claim 71, or the pharmaceutical composition of claims 72-73.

88. A method of wound healing in a tissue or skin in need thereof in an individual having an immune disorder, cancer, heart disease, kidney disease, lung disease, liver disease, infection, tissue injury, skin disorder, wound, or a combination thereof, wherein the tissue or skin is in need of wound healing due to toxicities induced by one or more therapies delivered to the individual for treatment of the immune disorder, cancer, heart disease, kidney disease, lung disease, liver disease, infection, tissue injury, skin disorder, wound, or a combination thereof, the method comprising the step of administering to the individual a therapeutically effective amount of exosomes produced by the method of any one of claims 1-37, the composition of claim 71, or the pharmaceutical composition of claims 72-73.

89. The method of any one of claims 84-88, wherein the one or more therapies received by the individual comprise chemotherapy, radiotherapy, or a combination thereof.

90. A method of regenerating and/or repairing tissue in need thereof in an individual having a tissue injury, wherein the tissue injury comprises inflammation, contusions, sprains, tendonitis, bursitis, stress injuries, strains, or a combination thereof, the method comprising the step of administering to the individual a therapeutically effective amount of exosomes produced by the method of any one of claims 1-37, the composition of claim 71, or the pharmaceutical composition of claims 72-73.

91. The method of any one of claims 86-90, wherein the tissue in need of regeneration and/or reparation comprises soft tissue, brain, lung, spleen, liver, heart, kidney, pancreas, intestine, testis, or bone.

92. A method of regenerating and/or repairing skin in need thereof in an individual having a skin disorder, wherein the skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, or a combination thereof, the method comprising the step of administering to the individual a therapeutically effective amount of exosomes produced by the method of any one of claims 1-37, the composition of claim 71, or the pharmaceutical composition of claims 72-73.

93. A method of wound healing in tissue or skin in need thereof in an individual having a tissue injury or skin disorder, wherein the tissue injury or skin disorder comprises inflammation, aging, skin cancer, acne, cold sores, blisters, seromas, hematomas, ulcers, carbuncles, warts, psoriasis, eczema, cellulitis, lupus, actinic keratosis, keratosis pilaris, shingles, hives, melasma, impetigo, sunburn, dermatitis, rosacea, thermal burns, chemical burns, electric burns, frostbite, cutaneous wound damage, contusions, cuts, lacerations, gashes, tears, punctures, scrapes, abrasions, scratches, bites, stings, bruises, pressure injuries, crush injuries, incisions, sprains, tendonitis, bursitis, stress injuries, strains, or a combination thereof, the method comprising the step of administering to the individual a therapeutically effective amount of exosomes produced by the method of any one of claims 1-37, the composition of claim 71, or the pharmaceutical composition of claims 72-73.

94. A method of reducing inflammation in an individual having an immune disorder, cancer, heart disease, kidney disease, lung disease, liver disease, infection, or a combination thereof, comprising the step of administering to the individual a therapeutically effective amount of exosomes produced by the method of any one of claims 1-37, the composition of claim 71, or the pharmaceutical composition of claims 72-73.

95. The method of any one of claims 74-94, wherein the exosomes cross the blood-brain barrier, the blood-tumor barrier, or a combination thereof.

96. The method of any one of claims 74-95, wherein the MSCs are autologous or allogeneic with respect to the individual.

97. The method of any one of claims 74-96, wherein the exosomes are administered via rectal, buccal, vaginal, subcutaneous, intranasal, intracutaneous, intravenous, intraperitoneal, intramuscular, intraarticular, intrasynovial, intrasternal, intrathecal, intralesional, or intracranial routes, or via an implanted reservoir.

98. The method of any one of claims 74-97, wherein the exosomes are administered in conjunction with at least one additional therapeutic agent.


SEQUENCE LISTING (11 sequences)

SEQ ID NO: 1 - DNA, 21 bp, Artificial Sequence
  Synthetic Polynucleotide
ccgtgacgtccaccgacgtaa

SEQ ID NO: 2 - DNA, 21 bp, Artificial Sequence
  Synthetic Polynucleotide
tgacgtccaccgacgtaaagg

SEQ ID NO: 3 - DNA, 21 bp, Artificial Sequence
  Synthetic Polynucleotide
gacgtccaccgacgtaaaggc

SEQ ID NO: 4 - DNA, 21 bp, Artificial Sequence
  Synthetic Polynucleotide
acgtccaccgacgtaaaggcg

SEQ ID NO: 5 - DNA, 21 bp, Artificial Sequence
  Synthetic Polynucleotide
cgtccaccgacgtaaaggcga

SEQ ID NO: 6 - DNA, 21 bp, Artificial Sequence
  Synthetic Polynucleotide
gtccaccgacgtaaaggcgac

SEQ ID NO: 7 - DNA, 21 bp, Artificial Sequence
  Synthetic Polynucleotide
tccaccgacgtaaaggcgaca

SEQ ID NO: 8 - DNA, 21 bp, Artificial Sequence
  Synthetic Polynucleotide
ccaccgacgtaaaggcgacac

SEQ ID NO: 9 - DNA, 21 bp, Artificial Sequence
  Synthetic Polynucleotide
caccgacgtaaaggcgacaca

SEQ ID NO: 10 - DNA, 21 bp, Artificial Sequence
  Synthetic Polynucleotide
ccgacgtaaaggcgacacagg

SEQ ID NO: 11 - DNA, 66 bp, Artificial Sequence
  Synthetic Polynucleotide
ggacctggaccgtgtccttaccgtgacgtccaccgacgtaaaggcgacacaggaggagaaccggga